SEQUENCE LISTING (480 sequences)

SEQ ID NO: 1 - DNA, 60 bp, Artificial Sequence
  Synthetic Polynucleotide
aatgatacggcgaccaccgagatctacactagatcgcacactctttccctacacgacgct

SEQ ID NO: 2 - DNA, 60 bp, Artificial Sequence
  Synthetic Polynucleotide
aatgatacggcgaccaccgagatctacacctctctatacactctttccctacacgacgct

SEQ ID NO: 3 - DNA, 60 bp, Artificial Sequence
  Synthetic Polynucleotide
aatgatacggcgaccaccgagatctacactatcctctacactctttccctacacgacgct

SEQ ID NO: 4 - DNA, 60 bp, Artificial Sequence
  Synthetic Polynucleotide
aatgatacggcgaccaccgagatctacacagagtagaacactctttccctacacgacgct

SEQ ID NO: 5 - DNA, 60 bp, Artificial Sequence
  Synthetic Polynucleotide
aatgatacggcgaccaccgagatctacacgtaaggagacactctttccctacacgacgct

SEQ ID NO: 6 - DNA, 60 bp, Artificial Sequence
  Synthetic Polynucleotide
aatgatacggcgaccaccgagatctacacactgcataacactctttccctacacgacgct

SEQ ID NO: 7 - DNA, 60 bp, Artificial Sequence
  Synthetic Polynucleotide
aatgatacggcgaccaccgagatctacacaaggagtaacactctttccctacacgacgct

SEQ ID NO: 8 - DNA, 60 bp, Artificial Sequence
  Synthetic Polynucleotide
aatgatacggcgaccaccgagatctacacctaagcctacactctttccctacacgacgct

SEQ ID NO: 9 - DNA, 60 bp, Artificial Sequence
  Synthetic Polynucleotide
aatgatacggcgaccaccgagatctacacgacattgtacactctttccctacacgacgct

SEQ ID NO: 10 - DNA, 60 bp, Artificial Sequence
  Synthetic Polynucleotide
aatgatacggcgaccaccgagatctacacactgatggacactctttccctacacgacgct

SEQ ID NO: 11 - DNA, 60 bp, Artificial Sequence
  Synthetic Polynucleotide
aatgatacggcgaccaccgagatctacacgtacctagacactctttccctacacgacgct

SEQ ID NO: 12 - DNA, 60 bp, Artificial Sequence
  Synthetic Polynucleotide
aatgatacggcgaccaccgagatctacaccagagctaacactctttccctacacgacgct

SEQ ID NO: 13 - DNA, 60 bp, Artificial Sequence
  Synthetic Polynucleotide
aatgatacggcgaccaccgagatctacaccatagtgaacactctttccctacacgacgct

SEQ ID NO: 14 - DNA, 60 bp, Artificial Sequence
  Synthetic Polynucleotide
aatgatacggcgaccaccgagatctacactacctagtacactctttccctacacgacgct

SEQ ID NO: 15 - DNA, 60 bp, Artificial Sequence
  Synthetic Polynucleotide
aatgatacggcgaccaccgagatctacaccgcgatatacactctttccctacacgacgct

SEQ ID NO: 16 - DNA, 60 bp, Artificial Sequence
  Synthetic Polynucleotide
aatgatacggcgaccaccgagatctacactggattgtacactctttccctacacgacgct

SEQ ID NO: 17 - DNA, 60 bp, Artificial Sequence
  Synthetic Polynucleotide
aatgatacggcgaccaccgagatctacacggacttccacactctttccctacacgacgct

SEQ ID NO: 18 - DNA, 60 bp, Artificial Sequence
  Synthetic Polynucleotide
aatgatacggcgaccaccgagatctacacggtatggcacactctttccctacacgacgct

SEQ ID NO: 19 - DNA, 60 bp, Artificial Sequence
  Synthetic Polynucleotide
aatgatacggcgaccaccgagatctacaccaatacgaacactctttccctacacgacgct

SEQ ID NO: 20 - DNA, 60 bp, Artificial Sequence
  Synthetic Polynucleotide
aatgatacggcgaccaccgagatctacacacaggtagacactctttccctacacgacgct

SEQ ID NO: 21 - DNA, 60 bp, Artificial Sequence
  Synthetic Polynucleotide
aatgatacggcgaccaccgagatctacactggagaggacactctttccctacacgacgct

SEQ ID NO: 22 - DNA, 60 bp, Artificial Sequence
  Synthetic Polynucleotide
aatgatacggcgaccaccgagatctacacttacgtggacactctttccctacacgacgct

SEQ ID NO: 23 - DNA, 60 bp, Artificial Sequence
  Synthetic Polynucleotide
aatgatacggcgaccaccgagatctacacatagccgaacactctttccctacacgacgct

SEQ ID NO: 24 - DNA, 60 bp, Artificial Sequence
  Synthetic Polynucleotide
aatgatacggcgaccaccgagatctacacgagtatctacactctttccctacacgacgct

SEQ ID NO: 25 - DNA, 60 bp, Artificial Sequence
  Synthetic Polynucleotide
aatgatacggcgaccaccgagatctacacgtcgtgtaacactctttccctacacgacgct

SEQ ID NO: 26 - DNA, 60 bp, Artificial Sequence
  Synthetic Polynucleotide
aatgatacggcgaccaccgagatctacactatccaagacactctttccctacacgacgct

SEQ ID NO: 27 - DNA, 60 bp, Artificial Sequence
  Synthetic Polynucleotide
aatgatacggcgaccaccgagatctacacagtcgctaacactctttccctacacgacgct

SEQ ID NO: 28 - DNA, 60 bp, Artificial Sequence
  Synthetic Polynucleotide
aatgatacggcgaccaccgagatctacacgacaggttacactctttccctacacgacgct

SEQ ID NO: 29 - DNA, 60 bp, Artificial Sequence
  Synthetic Polynucleotide
aatgatacggcgaccaccgagatctacacagattgacacactctttccctacacgacgct

SEQ ID NO: 30 - DNA, 60 bp, Artificial Sequence
  Synthetic Polynucleotide
aatgatacggcgaccaccgagatctacactatcaccgacactctttccctacacgacgct

SEQ ID NO: 31 - DNA, 60 bp, Artificial Sequence
  Synthetic Polynucleotide
aatgatacggcgaccaccgagatctacacataggctgacactctttccctacacgacgct

SEQ ID NO: 32 - DNA, 60 bp, Artificial Sequence
  Synthetic Polynucleotide
aatgatacggcgaccaccgagatctacacagtggcacacactctttccctacacgacgct

SEQ ID NO: 33 - DNA, 60 bp, Artificial Sequence
  Synthetic Polynucleotide
aatgatacggcgaccaccgagatctacacactcatctacactctttccctacacgacgct

SEQ ID NO: 34 - DNA, 60 bp, Artificial Sequence
  Synthetic Polynucleotide
aatgatacggcgaccaccgagatctacacgcagccatacactctttccctacacgacgct

SEQ ID NO: 35 - DNA, 60 bp, Artificial Sequence
  Synthetic Polynucleotide
aatgatacggcgaccaccgagatctacacttgcagtgacactctttccctacacgacgct

SEQ ID NO: 36 - DNA, 60 bp, Artificial Sequence
  Synthetic Polynucleotide
aatgatacggcgaccaccgagatctacaccgactgcaacactctttccctacacgacgct

SEQ ID NO: 37 - DNA, 60 bp, Artificial Sequence
  Synthetic Polynucleotide
aatgatacggcgaccaccgagatctacaccggtcaatacactctttccctacacgacgct

SEQ ID NO: 38 - DNA, 60 bp, Artificial Sequence
  Synthetic Polynucleotide
aatgatacggcgaccaccgagatctacacgctgctacacactctttccctacacgacgct

SEQ ID NO: 39 - DNA, 60 bp, Artificial Sequence
  Synthetic Polynucleotide
aatgatacggcgaccaccgagatctacacgcagtctaacactctttccctacacgacgct

SEQ ID NO: 40 - DNA, 60 bp, Artificial Sequence
  Synthetic Polynucleotide
aatgatacggcgaccaccgagatctacactggaccacacactctttccctacacgacgct

SEQ ID NO: 41 - DNA, 60 bp, Artificial Sequence
  Synthetic Polynucleotide
aatgatacggcgaccaccgagatctacacgtcacatcacactctttccctacacgacgct

SEQ ID NO: 42 - DNA, 60 bp, Artificial Sequence
  Synthetic Polynucleotide
aatgatacggcgaccaccgagatctacacgttgctgaacactctttccctacacgacgct

SEQ ID NO: 43 - DNA, 60 bp, Artificial Sequence
  Synthetic Polynucleotide
aatgatacggcgaccaccgagatctacacgagttagcacactctttccctacacgacgct

SEQ ID NO: 44 - DNA, 60 bp, Artificial Sequence
  Synthetic Polynucleotide
aatgatacggcgaccaccgagatctacacacgatcatacactctttccctacacgacgct

SEQ ID NO: 45 - DNA, 60 bp, Artificial Sequence
  Synthetic Polynucleotide
aatgatacggcgaccaccgagatctacaccgtcgtctacactctttccctacacgacgct

SEQ ID NO: 46 - DNA, 60 bp, Artificial Sequence
  Synthetic Polynucleotide
aatgatacggcgaccaccgagatctacacgacatgcgacactctttccctacacgacgct

SEQ ID NO: 47 - DNA, 60 bp, Artificial Sequence
  Synthetic Polynucleotide
aatgatacggcgaccaccgagatctacacgtgccataacactctttccctacacgacgct

SEQ ID NO: 48 - DNA, 60 bp, Artificial Sequence
  Synthetic Polynucleotide
aatgatacggcgaccaccgagatctacaccgctaggaacactctttccctacacgacgct

SEQ ID NO: 49 - DNA, 60 bp, Artificial Sequence
  Synthetic Polynucleotide
aatgatacggcgaccaccgagatctacaccgtaggtaacactctttccctacacgacgct

SEQ ID NO: 50 - DNA, 60 bp, Artificial Sequence
  Synthetic Polynucleotide
aatgatacggcgaccaccgagatctacacagctagcgacactctttccctacacgacgct

SEQ ID NO: 51 - DNA, 60 bp, Artificial Sequence
  Synthetic Polynucleotide
aatgatacggcgaccaccgagatctacactcctgtgcacactctttccctacacgacgct

SEQ ID NO: 52 - DNA, 60 bp, Artificial Sequence
  Synthetic Polynucleotide
aatgatacggcgaccaccgagatctacacgtaatctgacactctttccctacacgacgct

SEQ ID NO: 53 - DNA, 60 bp, Artificial Sequence
  Synthetic Polynucleotide
aatgatacggcgaccaccgagatctacacaacgtaggacactctttccctacacgacgct

SEQ ID NO: 54 - DNA, 60 bp, Artificial Sequence
  Synthetic Polynucleotide
aatgatacggcgaccaccgagatctacacttcctgttacactctttccctacacgacgct

SEQ ID NO: 55 - DNA, 60 bp, Artificial Sequence
  Synthetic Polynucleotide
aatgatacggcgaccaccgagatctacactgtccagtacactctttccctacacgacgct

SEQ ID NO: 56 - DNA, 60 bp, Artificial Sequence
  Synthetic Polynucleotide
aatgatacggcgaccaccgagatctacacacaaggcaacactctttccctacacgacgct

SEQ ID NO: 57 - DNA, 60 bp, Artificial Sequence
  Synthetic Polynucleotide
aatgatacggcgaccaccgagatctacacccttgaccacactctttccctacacgacgct

SEQ ID NO: 58 - DNA, 60 bp, Artificial Sequence
  Synthetic Polynucleotide
aatgatacggcgaccaccgagatctacaccgcttgtgacactctttccctacacgacgct

SEQ ID NO: 59 - DNA, 60 bp, Artificial Sequence
  Synthetic Polynucleotide
aatgatacggcgaccaccgagatctacactccaagcgacactctttccctacacgacgct

SEQ ID NO: 60 - DNA, 60 bp, Artificial Sequence
  Synthetic Polynucleotide
aatgatacggcgaccaccgagatctacacctagtgacacactctttccctacacgacgct

SEQ ID NO: 61 - DNA, 60 bp, Artificial Sequence
  Synthetic Polynucleotide
aatgatacggcgaccaccgagatctacacagaaccgtacactctttccctacacgacgct

SEQ ID NO: 62 - DNA, 60 bp, Artificial Sequence
  Synthetic Polynucleotide
aatgatacggcgaccaccgagatctacactaattgcaacactctttccctacacgacgct

SEQ ID NO: 63 - DNA, 60 bp, Artificial Sequence
  Synthetic Polynucleotide
aatgatacggcgaccaccgagatctacacctagtacaacactctttccctacacgacgct

SEQ ID NO: 64 - DNA, 60 bp, Artificial Sequence
  Synthetic Polynucleotide
aatgatacggcgaccaccgagatctacacgctatatcacactctttccctacacgacgct

SEQ ID NO: 65 - DNA, 60 bp, Artificial Sequence
  Synthetic Polynucleotide
aatgatacggcgaccaccgagatctacaccaatcggcacactctttccctacacgacgct

SEQ ID NO: 66 - DNA, 60 bp, Artificial Sequence
  Synthetic Polynucleotide
aatgatacggcgaccaccgagatctacaccgatatcaacactctttccctacacgacgct

SEQ ID NO: 67 - DNA, 60 bp, Artificial Sequence
  Synthetic Polynucleotide
aatgatacggcgaccaccgagatctacaccagtcaggacactctttccctacacgacgct

SEQ ID NO: 68 - DNA, 60 bp, Artificial Sequence
  Synthetic Polynucleotide
aatgatacggcgaccaccgagatctacacgtaataatacactctttccctacacgacgct

SEQ ID NO: 69 - DNA, 60 bp, Artificial Sequence
  Synthetic Polynucleotide
aatgatacggcgaccaccgagatctacacggagagatacactctttccctacacgacgct

SEQ ID NO: 70 - DNA, 60 bp, Artificial Sequence
  Synthetic Polynucleotide
aatgatacggcgaccaccgagatctacacctctcataacactctttccctacacgacgct

SEQ ID NO: 71 - DNA, 60 bp, Artificial Sequence
  Synthetic Polynucleotide
aatgatacggcgaccaccgagatctacaccagcgactacactctttccctacacgacgct

SEQ ID NO: 72 - DNA, 60 bp, Artificial Sequence
  Synthetic Polynucleotide
aatgatacggcgaccaccgagatctacacggccaaggacactctttccctacacgacgct

SEQ ID NO: 73 - DNA, 60 bp, Artificial Sequence
  Synthetic Polynucleotide
aatgatacggcgaccaccgagatctacacgcatatgcacactctttccctacacgacgct

SEQ ID NO: 74 - DNA, 60 bp, Artificial Sequence
  Synthetic Polynucleotide
aatgatacggcgaccaccgagatctacacactaggatacactctttccctacacgacgct

SEQ ID NO: 75 - DNA, 60 bp, Artificial Sequence
  Synthetic Polynucleotide
aatgatacggcgaccaccgagatctacacccttacctacactctttccctacacgacgct

SEQ ID NO: 76 - DNA, 60 bp, Artificial Sequence
  Synthetic Polynucleotide
aatgatacggcgaccaccgagatctacactgttgacgacactctttccctacacgacgct

SEQ ID NO: 77 - DNA, 60 bp, Artificial Sequence
  Synthetic Polynucleotide
aatgatacggcgaccaccgagatctacactacagttaacactctttccctacacgacgct

SEQ ID NO: 78 - DNA, 60 bp, Artificial Sequence
  Synthetic Polynucleotide
aatgatacggcgaccaccgagatctacacttgttacgacactctttccctacacgacgct

SEQ ID NO: 79 - DNA, 60 bp, Artificial Sequence
  Synthetic Polynucleotide
aatgatacggcgaccaccgagatctacactcgtgttgacactctttccctacacgacgct

SEQ ID NO: 80 - DNA, 60 bp, Artificial Sequence
  Synthetic Polynucleotide
aatgatacggcgaccaccgagatctacacagtcaatgacactctttccctacacgacgct

SEQ ID NO: 81 - DNA, 60 bp, Artificial Sequence
  Synthetic Polynucleotide
aatgatacggcgaccaccgagatctacactctgtagaacactctttccctacacgacgct

SEQ ID NO: 82 - DNA, 60 bp, Artificial Sequence
  Synthetic Polynucleotide
aatgatacggcgaccaccgagatctacacgacaacgaacactctttccctacacgacgct

SEQ ID NO: 83 - DNA, 60 bp, Artificial Sequence
  Synthetic Polynucleotide
aatgatacggcgaccaccgagatctacacccatggctacactctttccctacacgacgct

SEQ ID NO: 84 - DNA, 60 bp, Artificial Sequence
  Synthetic Polynucleotide
aatgatacggcgaccaccgagatctacactgactctgacactctttccctacacgacgct

SEQ ID NO: 85 - DNA, 60 bp, Artificial Sequence
  Synthetic Polynucleotide
aatgatacggcgaccaccgagatctacacaacgaggcacactctttccctacacgacgct

SEQ ID NO: 86 - DNA, 60 bp, Artificial Sequence
  Synthetic Polynucleotide
aatgatacggcgaccaccgagatctacaccagaaggtacactctttccctacacgacgct

SEQ ID NO: 87 - DNA, 60 bp, Artificial Sequence
  Synthetic Polynucleotide
aatgatacggcgaccaccgagatctacactgaagtcaacactctttccctacacgacgct

SEQ ID NO: 88 - DNA, 60 bp, Artificial Sequence
  Synthetic Polynucleotide
aatgatacggcgaccaccgagatctacacatgttcctacactctttccctacacgacgct

SEQ ID NO: 89 - DNA, 60 bp, Artificial Sequence
  Synthetic Polynucleotide
aatgatacggcgaccaccgagatctacacaagtggctacactctttccctacacgacgct

SEQ ID NO: 90 - DNA, 60 bp, Artificial Sequence
  Synthetic Polynucleotide
aatgatacggcgaccaccgagatctacacggtacaatacactctttccctacacgacgct

SEQ ID NO: 91 - DNA, 60 bp, Artificial Sequence
  Synthetic Polynucleotide
aatgatacggcgaccaccgagatctacacacaagtgcacactctttccctacacgacgct

SEQ ID NO: 92 - DNA, 60 bp, Artificial Sequence
  Synthetic Polynucleotide
aatgatacggcgaccaccgagatctacactcacggtgacactctttccctacacgacgct

SEQ ID NO: 93 - DNA, 60 bp, Artificial Sequence
  Synthetic Polynucleotide
aatgatacggcgaccaccgagatctacacttgcgttaacactctttccctacacgacgct

SEQ ID NO: 94 - DNA, 60 bp, Artificial Sequence
  Synthetic Polynucleotide
aatgatacggcgaccaccgagatctacacttgtagccacactctttccctacacgacgct

SEQ ID NO: 95 - DNA, 60 bp, Artificial Sequence
  Synthetic Polynucleotide
aatgatacggcgaccaccgagatctacactcaccggaacactctttccctacacgacgct

SEQ ID NO: 96 - DNA, 60 bp, Artificial Sequence
  Synthetic Polynucleotide
aatgatacggcgaccaccgagatctacaccgcgcaagacactctttccctacacgacgct

SEQ ID NO: 97 - DNA, 65 bp, Artificial Sequence
  Synthetic Polynucleotide
catacgatttaggtgacactatagatagatcgcagatcggaagagcgtcgtgtagggaaagagac

SEQ ID NO: 98 - DNA, 65 bp, Artificial Sequence
  Synthetic Polynucleotide
catacgatttaggtgacactatagactctctatagatcggaagagcgtcgtgtagggaaagagac

SEQ ID NO: 99 - DNA, 65 bp, Artificial Sequence
  Synthetic Polynucleotide
catacgatttaggtgacactatagatatcctctagatcggaagagcgtcgtgtagggaaagagac

SEQ ID NO: 100 - DNA, 65 bp, Artificial Sequence
  Synthetic Polynucleotide
catacgatttaggtgacactatagaagagtagaagatcggaagagcgtcgtgtagggaaagagac

SEQ ID NO: 101 - DNA, 65 bp, Artificial Sequence
  Synthetic Polynucleotide
catacgatttaggtgacactatagagtaaggagagatcggaagagcgtcgtgtagggaaagagac

SEQ ID NO: 102 - DNA, 65 bp, Artificial Sequence
  Synthetic Polynucleotide
catacgatttaggtgacactatagaactgcataagatcggaagagcgtcgtgtagggaaagagac

SEQ ID NO: 103 - DNA, 65 bp, Artificial Sequence
  Synthetic Polynucleotide
catacgatttaggtgacactatagaaaggagtaagatcggaagagcgtcgtgtagggaaagagac

SEQ ID NO: 104 - DNA, 65 bp, Artificial Sequence
  Synthetic Polynucleotide
catacgatttaggtgacactatagactaagcctagatcggaagagcgtcgtgtagggaaagagac

SEQ ID NO: 105 - DNA, 65 bp, Artificial Sequence
  Synthetic Polynucleotide
catacgatttaggtgacactatagagacattgtagatcggaagagcgtcgtgtagggaaagagac

SEQ ID NO: 106 - DNA, 65 bp, Artificial Sequence
  Synthetic Polynucleotide
catacgatttaggtgacactatagaactgatggagatcggaagagcgtcgtgtagggaaagagac

SEQ ID NO: 107 - DNA, 65 bp, Artificial Sequence
  Synthetic Polynucleotide
catacgatttaggtgacactatagagtacctagagatcggaagagcgtcgtgtagggaaagagac

SEQ ID NO: 108 - DNA, 65 bp, Artificial Sequence
  Synthetic Polynucleotide
catacgatttaggtgacactatagacagagctaagatcggaagagcgtcgtgtagggaaagagac

SEQ ID NO: 109 - DNA, 65 bp, Artificial Sequence
  Synthetic Polynucleotide
catacgatttaggtgacactatagacatagtgaagatcggaagagcgtcgtgtagggaaagagac

SEQ ID NO: 110 - DNA, 65 bp, Artificial Sequence
  Synthetic Polynucleotide
catacgatttaggtgacactatagatacctagtagatcggaagagcgtcgtgtagggaaagagac

SEQ ID NO: 111 - DNA, 65 bp, Artificial Sequence
  Synthetic Polynucleotide
catacgatttaggtgacactatagacgcgatatagatcggaagagcgtcgtgtagggaaagagac

SEQ ID NO: 112 - DNA, 65 bp, Artificial Sequence
  Synthetic Polynucleotide
catacgatttaggtgacactatagatggattgtagatcggaagagcgtcgtgtagggaaagagac

SEQ ID NO: 113 - DNA, 65 bp, Artificial Sequence
  Synthetic Polynucleotide
catacgatttaggtgacactatagaggacttccagatcggaagagcgtcgtgtagggaaagagac

SEQ ID NO: 114 - DNA, 65 bp, Artificial Sequence
  Synthetic Polynucleotide
catacgatttaggtgacactatagaggtatggcagatcggaagagcgtcgtgtagggaaagagac

SEQ ID NO: 115 - DNA, 65 bp, Artificial Sequence
  Synthetic Polynucleotide
catacgatttaggtgacactatagacaatacgaagatcggaagagcgtcgtgtagggaaagagac

SEQ ID NO: 116 - DNA, 65 bp, Artificial Sequence
  Synthetic Polynucleotide
catacgatttaggtgacactatagaacaggtagagatcggaagagcgtcgtgtagggaaagagac

SEQ ID NO: 117 - DNA, 65 bp, Artificial Sequence
  Synthetic Polynucleotide
catacgatttaggtgacactatagatggagaggagatcggaagagcgtcgtgtagggaaagagac

SEQ ID NO: 118 - DNA, 65 bp, Artificial Sequence
  Synthetic Polynucleotide
catacgatttaggtgacactatagattacgtggagatcggaagagcgtcgtgtagggaaagagac

SEQ ID NO: 119 - DNA, 65 bp, Artificial Sequence
  Synthetic Polynucleotide
catacgatttaggtgacactatagaatagccgaagatcggaagagcgtcgtgtagggaaagagac

SEQ ID NO: 120 - DNA, 65 bp, Artificial Sequence
  Synthetic Polynucleotide
catacgatttaggtgacactatagagagtatctagatcggaagagcgtcgtgtagggaaagagac

SEQ ID NO: 121 - DNA, 65 bp, Artificial Sequence
  Synthetic Polynucleotide
catacgatttaggtgacactatagagtcgtgtaagatcggaagagcgtcgtgtagggaaagagac

SEQ ID NO: 122 - DNA, 65 bp, Artificial Sequence
  Synthetic Polynucleotide
catacgatttaggtgacactatagatatccaagagatcggaagagcgtcgtgtagggaaagagac

SEQ ID NO: 123 - DNA, 65 bp, Artificial Sequence
  Synthetic Polynucleotide
catacgatttaggtgacactatagaagtcgctaagatcggaagagcgtcgtgtagggaaagagac

SEQ ID NO: 124 - DNA, 65 bp, Artificial Sequence
  Synthetic Polynucleotide
catacgatttaggtgacactatagagacaggttagatcggaagagcgtcgtgtagggaaagagac

SEQ ID NO: 125 - DNA, 65 bp, Artificial Sequence
  Synthetic Polynucleotide
catacgatttaggtgacactatagaagattgacagatcggaagagcgtcgtgtagggaaagagac

SEQ ID NO: 126 - DNA, 65 bp, Artificial Sequence
  Synthetic Polynucleotide
catacgatttaggtgacactatagatatcaccgagatcggaagagcgtcgtgtagggaaagagac

SEQ ID NO: 127 - DNA, 65 bp, Artificial Sequence
  Synthetic Polynucleotide
catacgatttaggtgacactatagaataggctgagatcggaagagcgtcgtgtagggaaagagac

SEQ ID NO: 128 - DNA, 65 bp, Artificial Sequence
  Synthetic Polynucleotide
catacgatttaggtgacactatagaagtggcacagatcggaagagcgtcgtgtagggaaagagac

SEQ ID NO: 129 - DNA, 65 bp, Artificial Sequence
  Synthetic Polynucleotide
catacgatttaggtgacactatagaactcatctagatcggaagagcgtcgtgtagggaaagagac

SEQ ID NO: 130 - DNA, 65 bp, Artificial Sequence
  Synthetic Polynucleotide
catacgatttaggtgacactatagagcagccatagatcggaagagcgtcgtgtagggaaagagac

SEQ ID NO: 131 - DNA, 65 bp, Artificial Sequence
  Synthetic Polynucleotide
catacgatttaggtgacactatagattgcagtgagatcggaagagcgtcgtgtagggaaagagac

SEQ ID NO: 132 - DNA, 65 bp, Artificial Sequence
  Synthetic Polynucleotide
catacgatttaggtgacactatagacgactgcaagatcggaagagcgtcgtgtagggaaagagac

SEQ ID NO: 133 - DNA, 65 bp, Artificial Sequence
  Synthetic Polynucleotide
catacgatttaggtgacactatagacggtcaatagatcggaagagcgtcgtgtagggaaagagac

SEQ ID NO: 134 - DNA, 65 bp, Artificial Sequence
  Synthetic Polynucleotide
catacgatttaggtgacactatagagctgctacagatcggaagagcgtcgtgtagggaaagagac

SEQ ID NO: 135 - DNA, 65 bp, Artificial Sequence
  Synthetic Polynucleotide
catacgatttaggtgacactatagagcagtctaagatcggaagagcgtcgtgtagggaaagagac

SEQ ID NO: 136 - DNA, 65 bp, Artificial Sequence
  Synthetic Polynucleotide
catacgatttaggtgacactatagatggaccacagatcggaagagcgtcgtgtagggaaagagac

SEQ ID NO: 137 - DNA, 65 bp, Artificial Sequence
  Synthetic Polynucleotide
catacgatttaggtgacactatagagtcacatcagatcggaagagcgtcgtgtagggaaagagac

SEQ ID NO: 138 - DNA, 65 bp, Artificial Sequence
  Synthetic Polynucleotide
catacgatttaggtgacactatagagttgctgaagatcggaagagcgtcgtgtagggaaagagac

SEQ ID NO: 139 - DNA, 65 bp, Artificial Sequence
  Synthetic Polynucleotide
catacgatttaggtgacactatagagagttagcagatcggaagagcgtcgtgtagggaaagagac

SEQ ID NO: 140 - DNA, 65 bp, Artificial Sequence
  Synthetic Polynucleotide
catacgatttaggtgacactatagaacgatcatagatcggaagagcgtcgtgtagggaaagagac

SEQ ID NO: 141 - DNA, 65 bp, Artificial Sequence
  Synthetic Polynucleotide
catacgatttaggtgacactatagacgtcgtctagatcggaagagcgtcgtgtagggaaagagac

SEQ ID NO: 142 - DNA, 65 bp, Artificial Sequence
  Synthetic Polynucleotide
catacgatttaggtgacactatagagacatgcgagatcggaagagcgtcgtgtagggaaagagac

SEQ ID NO: 143 - DNA, 65 bp, Artificial Sequence
  Synthetic Polynucleotide
catacgatttaggtgacactatagagtgccataagatcggaagagcgtcgtgtagggaaagagac

SEQ ID NO: 144 - DNA, 65 bp, Artificial Sequence
  Synthetic Polynucleotide
catacgatttaggtgacactatagacgctaggaagatcggaagagcgtcgtgtagggaaagagac

SEQ ID NO: 145 - DNA, 65 bp, Artificial Sequence
  Synthetic Polynucleotide
catacgatttaggtgacactatagacgtaggtaagatcggaagagcgtcgtgtagggaaagagac

SEQ ID NO: 146 - DNA, 65 bp, Artificial Sequence
  Synthetic Polynucleotide
catacgatttaggtgacactatagaagctagcgagatcggaagagcgtcgtgtagggaaagagac

SEQ ID NO: 147 - DNA, 65 bp, Artificial Sequence
  Synthetic Polynucleotide
catacgatttaggtgacactatagatcctgtgcagatcggaagagcgtcgtgtagggaaagagac

SEQ ID NO: 148 - DNA, 65 bp, Artificial Sequence
  Synthetic Polynucleotide
catacgatttaggtgacactatagagtaatctgagatcggaagagcgtcgtgtagggaaagagac

SEQ ID NO: 149 - DNA, 65 bp, Artificial Sequence
  Synthetic Polynucleotide
catacgatttaggtgacactatagaaacgtaggagatcggaagagcgtcgtgtagggaaagagac

SEQ ID NO: 150 - DNA, 65 bp, Artificial Sequence
  Synthetic Polynucleotide
catacgatttaggtgacactatagattcctgttagatcggaagagcgtcgtgtagggaaagagac

SEQ ID NO: 151 - DNA, 65 bp, Artificial Sequence
  Synthetic Polynucleotide
catacgatttaggtgacactatagatgtccagtagatcggaagagcgtcgtgtagggaaagagac

SEQ ID NO: 152 - DNA, 65 bp, Artificial Sequence
  Synthetic Polynucleotide
catacgatttaggtgacactatagaacaaggcaagatcggaagagcgtcgtgtagggaaagagac

SEQ ID NO: 153 - DNA, 65 bp, Artificial Sequence
  Synthetic Polynucleotide
catacgatttaggtgacactatagaccttgaccagatcggaagagcgtcgtgtagggaaagagac

SEQ ID NO: 154 - DNA, 65 bp, Artificial Sequence
  Synthetic Polynucleotide
catacgatttaggtgacactatagacgcttgtgagatcggaagagcgtcgtgtagggaaagagac

SEQ ID NO: 155 - DNA, 65 bp, Artificial Sequence
  Synthetic Polynucleotide
catacgatttaggtgacactatagatccaagcgagatcggaagagcgtcgtgtagggaaagagac

SEQ ID NO: 156 - DNA, 65 bp, Artificial Sequence
  Synthetic Polynucleotide
catacgatttaggtgacactatagactagtgacagatcggaagagcgtcgtgtagggaaagagac

SEQ ID NO: 157 - DNA, 65 bp, Artificial Sequence
  Synthetic Polynucleotide
catacgatttaggtgacactatagaagaaccgtagatcggaagagcgtcgtgtagggaaagagac

SEQ ID NO: 158 - DNA, 65 bp, Artificial Sequence
  Synthetic Polynucleotide
catacgatttaggtgacactatagataattgcaagatcggaagagcgtcgtgtagggaaagagac

SEQ ID NO: 159 - DNA, 65 bp, Artificial Sequence
  Synthetic Polynucleotide
catacgatttaggtgacactatagactagtacaagatcggaagagcgtcgtgtagggaaagagac

SEQ ID NO: 160 - DNA, 65 bp, Artificial Sequence
  Synthetic Polynucleotide
catacgatttaggtgacactatagagctatatcagatcggaagagcgtcgtgtagggaaagagac

SEQ ID NO: 161 - DNA, 65 bp, Artificial Sequence
  Synthetic Polynucleotide
catacgatttaggtgacactatagacaatcggcagatcggaagagcgtcgtgtagggaaagagac

SEQ ID NO: 162 - DNA, 65 bp, Artificial Sequence
  Synthetic Polynucleotide
catacgatttaggtgacactatagacgatatcaagatcggaagagcgtcgtgtagggaaagagac

SEQ ID NO: 163 - DNA, 65 bp, Artificial Sequence
  Synthetic Polynucleotide
catacgatttaggtgacactatagacagtcaggagatcggaagagcgtcgtgtagggaaagagac

SEQ ID NO: 164 - DNA, 65 bp, Artificial Sequence
  Synthetic Polynucleotide
catacgatttaggtgacactatagagtaataatagatcggaagagcgtcgtgtagggaaagagac

SEQ ID NO: 165 - DNA, 65 bp, Artificial Sequence
  Synthetic Polynucleotide
catacgatttaggtgacactatagaggagagatagatcggaagagcgtcgtgtagggaaagagac

SEQ ID NO: 166 - DNA, 65 bp, Artificial Sequence
  Synthetic Polynucleotide
catacgatttaggtgacactatagactctcataagatcggaagagcgtcgtgtagggaaagagac

SEQ ID NO: 167 - DNA, 65 bp, Artificial Sequence
  Synthetic Polynucleotide
catacgatttaggtgacactatagacagcgactagatcggaagagcgtcgtgtagggaaagagac

SEQ ID NO: 168 - DNA, 65 bp, Artificial Sequence
  Synthetic Polynucleotide
catacgatttaggtgacactatagaggccaaggagatcggaagagcgtcgtgtagggaaagagac

SEQ ID NO: 169 - DNA, 65 bp, Artificial Sequence
  Synthetic Polynucleotide
catacgatttaggtgacactatagagcatatgcagatcggaagagcgtcgtgtagggaaagagac

SEQ ID NO: 170 - DNA, 65 bp, Artificial Sequence
  Synthetic Polynucleotide
catacgatttaggtgacactatagaactaggatagatcggaagagcgtcgtgtagggaaagagac

SEQ ID NO: 171 - DNA, 65 bp, Artificial Sequence
  Synthetic Polynucleotide
catacgatttaggtgacactatagaccttacctagatcggaagagcgtcgtgtagggaaagagac

SEQ ID NO: 172 - DNA, 65 bp, Artificial Sequence
  Synthetic Polynucleotide
catacgatttaggtgacactatagatgttgacgagatcggaagagcgtcgtgtagggaaagagac

SEQ ID NO: 173 - DNA, 65 bp, Artificial Sequence
  Synthetic Polynucleotide
catacgatttaggtgacactatagatacagttaagatcggaagagcgtcgtgtagggaaagagac

SEQ ID NO: 174 - DNA, 65 bp, Artificial Sequence
  Synthetic Polynucleotide
catacgatttaggtgacactatagattgttacgagatcggaagagcgtcgtgtagggaaagagac

SEQ ID NO: 175 - DNA, 65 bp, Artificial Sequence
  Synthetic Polynucleotide
catacgatttaggtgacactatagatcgtgttgagatcggaagagcgtcgtgtagggaaagagac

SEQ ID NO: 176 - DNA, 65 bp, Artificial Sequence
  Synthetic Polynucleotide
catacgatttaggtgacactatagaagtcaatgagatcggaagagcgtcgtgtagggaaagagac

SEQ ID NO: 177 - DNA, 65 bp, Artificial Sequence
  Synthetic Polynucleotide
catacgatttaggtgacactatagatctgtagaagatcggaagagcgtcgtgtagggaaagagac

SEQ ID NO: 178 - DNA, 65 bp, Artificial Sequence
  Synthetic Polynucleotide
catacgatttaggtgacactatagagacaacgaagatcggaagagcgtcgtgtagggaaagagac

SEQ ID NO: 179 - DNA, 65 bp, Artificial Sequence
  Synthetic Polynucleotide
catacgatttaggtgacactatagaccatggctagatcggaagagcgtcgtgtagggaaagagac

SEQ ID NO: 180 - DNA, 65 bp, Artificial Sequence
  Synthetic Polynucleotide
catacgatttaggtgacactatagatgactctgagatcggaagagcgtcgtgtagggaaagagac

SEQ ID NO: 181 - DNA, 65 bp, Artificial Sequence
  Synthetic Polynucleotide
catacgatttaggtgacactatagaaacgaggcagatcggaagagcgtcgtgtagggaaagagac

SEQ ID NO: 182 - DNA, 65 bp, Artificial Sequence
  Synthetic Polynucleotide
catacgatttaggtgacactatagacagaaggtagatcggaagagcgtcgtgtagggaaagagac

SEQ ID NO: 183 - DNA, 65 bp, Artificial Sequence
  Synthetic Polynucleotide
catacgatttaggtgacactatagatgaagtcaagatcggaagagcgtcgtgtagggaaagagac

SEQ ID NO: 184 - DNA, 65 bp, Artificial Sequence
  Synthetic Polynucleotide
catacgatttaggtgacactatagaatgttcctagatcggaagagcgtcgtgtagggaaagagac

SEQ ID NO: 185 - DNA, 65 bp, Artificial Sequence
  Synthetic Polynucleotide
catacgatttaggtgacactatagaaagtggctagatcggaagagcgtcgtgtagggaaagagac

SEQ ID NO: 186 - DNA, 65 bp, Artificial Sequence
  Synthetic Polynucleotide
catacgatttaggtgacactatagaggtacaatagatcggaagagcgtcgtgtagggaaagagac

SEQ ID NO: 187 - DNA, 65 bp, Artificial Sequence
  Synthetic Polynucleotide
catacgatttaggtgacactatagaacaagtgcagatcggaagagcgtcgtgtagggaaagagac

SEQ ID NO: 188 - DNA, 65 bp, Artificial Sequence
  Synthetic Polynucleotide
catacgatttaggtgacactatagatcacggtgagatcggaagagcgtcgtgtagggaaagagac

SEQ ID NO: 189 - DNA, 65 bp, Artificial Sequence
  Synthetic Polynucleotide
catacgatttaggtgacactatagattgcgttaagatcggaagagcgtcgtgtagggaaagagac

SEQ ID NO: 190 - DNA, 65 bp, Artificial Sequence
  Synthetic Polynucleotide
catacgatttaggtgacactatagattgtagccagatcggaagagcgtcgtgtagggaaagagac

SEQ ID NO: 191 - DNA, 65 bp, Artificial Sequence
  Synthetic Polynucleotide
catacgatttaggtgacactatagatcaccggaagatcggaagagcgtcgtgtagggaaagagac

SEQ ID NO: 192 - DNA, 65 bp, Artificial Sequence
  Synthetic Polynucleotide
catacgatttaggtgacactatagacgcgcaagagatcggaagagcgtcgtgtagggaaagagac

SEQ ID NO: 193 - DNA, 58 bp, Artificial Sequence
  Synthetic Polynucleotide
tctatagtgtcacctaaatcgtatgtagatcgccaggagcaaggtgagatgacaggag

SEQ ID NO: 194 - DNA, 58 bp, Artificial Sequence
  Synthetic Polynucleotide
tctatagtgtcacctaaatcgtatgctctctatcaggagcaaggtgagatgacaggag

SEQ ID NO: 195 - DNA, 58 bp, Artificial Sequence
  Synthetic Polynucleotide
tctatagtgtcacctaaatcgtatgtatcctctcaggagcaaggtgagatgacaggag

SEQ ID NO: 196 - DNA, 58 bp, Artificial Sequence
  Synthetic Polynucleotide
tctatagtgtcacctaaatcgtatgagagtagacaggagcaaggtgagatgacaggag

SEQ ID NO: 197 - DNA, 58 bp, Artificial Sequence
  Synthetic Polynucleotide
tctatagtgtcacctaaatcgtatggtaaggagcaggagcaaggtgagatgacaggag

SEQ ID NO: 198 - DNA, 58 bp, Artificial Sequence
  Synthetic Polynucleotide
tctatagtgtcacctaaatcgtatgactgcatacaggagcaaggtgagatgacaggag

SEQ ID NO: 199 - DNA, 58 bp, Artificial Sequence
  Synthetic Polynucleotide
tctatagtgtcacctaaatcgtatgaaggagtacaggagcaaggtgagatgacaggag

SEQ ID NO: 200 - DNA, 58 bp, Artificial Sequence
  Synthetic Polynucleotide
tctatagtgtcacctaaatcgtatgctaagcctcaggagcaaggtgagatgacaggag

SEQ ID NO: 201 - DNA, 58 bp, Artificial Sequence
  Synthetic Polynucleotide
tctatagtgtcacctaaatcgtatggacattgtcaggagcaaggtgagatgacaggag

SEQ ID NO: 202 - DNA, 58 bp, Artificial Sequence
  Synthetic Polynucleotide
tctatagtgtcacctaaatcgtatgactgatggcaggagcaaggtgagatgacaggag

SEQ ID NO: 203 - DNA, 58 bp, Artificial Sequence
  Synthetic Polynucleotide
tctatagtgtcacctaaatcgtatggtacctagcaggagcaaggtgagatgacaggag

SEQ ID NO: 204 - DNA, 58 bp, Artificial Sequence
  Synthetic Polynucleotide
tctatagtgtcacctaaatcgtatgcagagctacaggagcaaggtgagatgacaggag

SEQ ID NO: 205 - DNA, 58 bp, Artificial Sequence
  Synthetic Polynucleotide
tctatagtgtcacctaaatcgtatgcatagtgacaggagcaaggtgagatgacaggag

SEQ ID NO: 206 - DNA, 58 bp, Artificial Sequence
  Synthetic Polynucleotide
tctatagtgtcacctaaatcgtatgtacctagtcaggagcaaggtgagatgacaggag

SEQ ID NO: 207 - DNA, 58 bp, Artificial Sequence
  Synthetic Polynucleotide
tctatagtgtcacctaaatcgtatgcgcgatatcaggagcaaggtgagatgacaggag

SEQ ID NO: 208 - DNA, 58 bp, Artificial Sequence
  Synthetic Polynucleotide
tctatagtgtcacctaaatcgtatgtggattgtcaggagcaaggtgagatgacaggag

SEQ ID NO: 209 - DNA, 58 bp, Artificial Sequence
  Synthetic Polynucleotide
tctatagtgtcacctaaatcgtatgggacttcccaggagcaaggtgagatgacaggag

SEQ ID NO: 210 - DNA, 58 bp, Artificial Sequence
  Synthetic Polynucleotide
tctatagtgtcacctaaatcgtatgggtatggccaggagcaaggtgagatgacaggag

SEQ ID NO: 211 - DNA, 58 bp, Artificial Sequence
  Synthetic Polynucleotide
tctatagtgtcacctaaatcgtatgcaatacgacaggagcaaggtgagatgacaggag

SEQ ID NO: 212 - DNA, 58 bp, Artificial Sequence
  Synthetic Polynucleotide
tctatagtgtcacctaaatcgtatgacaggtagcaggagcaaggtgagatgacaggag

SEQ ID NO: 213 - DNA, 58 bp, Artificial Sequence
  Synthetic Polynucleotide
tctatagtgtcacctaaatcgtatgtggagaggcaggagcaaggtgagatgacaggag

SEQ ID NO: 214 - DNA, 58 bp, Artificial Sequence
  Synthetic Polynucleotide
tctatagtgtcacctaaatcgtatgttacgtggcaggagcaaggtgagatgacaggag

SEQ ID NO: 215 - DNA, 58 bp, Artificial Sequence
  Synthetic Polynucleotide
tctatagtgtcacctaaatcgtatgatagccgacaggagcaaggtgagatgacaggag

SEQ ID NO: 216 - DNA, 58 bp, Artificial Sequence
  Synthetic Polynucleotide
tctatagtgtcacctaaatcgtatggagtatctcaggagcaaggtgagatgacaggag

SEQ ID NO: 217 - DNA, 58 bp, Artificial Sequence
  Synthetic Polynucleotide
tctatagtgtcacctaaatcgtatggtcgtgtacaggagcaaggtgagatgacaggag

SEQ ID NO: 218 - DNA, 58 bp, Artificial Sequence
  Synthetic Polynucleotide
tctatagtgtcacctaaatcgtatgtatccaagcaggagcaaggtgagatgacaggag

SEQ ID NO: 219 - DNA, 58 bp, Artificial Sequence
  Synthetic Polynucleotide
tctatagtgtcacctaaatcgtatgagtcgctacaggagcaaggtgagatgacaggag

SEQ ID NO: 220 - DNA, 58 bp, Artificial Sequence
  Synthetic Polynucleotide
tctatagtgtcacctaaatcgtatggacaggttcaggagcaaggtgagatgacaggag

SEQ ID NO: 221 - DNA, 58 bp, Artificial Sequence
  Synthetic Polynucleotide
tctatagtgtcacctaaatcgtatgagattgaccaggagcaaggtgagatgacaggag

SEQ ID NO: 222 - DNA, 58 bp, Artificial Sequence
  Synthetic Polynucleotide
tctatagtgtcacctaaatcgtatgtatcaccgcaggagcaaggtgagatgacaggag

SEQ ID NO: 223 - DNA, 58 bp, Artificial Sequence
  Synthetic Polynucleotide
tctatagtgtcacctaaatcgtatgataggctgcaggagcaaggtgagatgacaggag

SEQ ID NO: 224 - DNA, 58 bp, Artificial Sequence
  Synthetic Polynucleotide
tctatagtgtcacctaaatcgtatgagtggcaccaggagcaaggtgagatgacaggag

SEQ ID NO: 225 - DNA, 58 bp, Artificial Sequence
  Synthetic Polynucleotide
tctatagtgtcacctaaatcgtatgactcatctcaggagcaaggtgagatgacaggag

SEQ ID NO: 226 - DNA, 58 bp, Artificial Sequence
  Synthetic Polynucleotide
tctatagtgtcacctaaatcgtatggcagccatcaggagcaaggtgagatgacaggag

SEQ ID NO: 227 - DNA, 58 bp, Artificial Sequence
  Synthetic Polynucleotide
tctatagtgtcacctaaatcgtatgttgcagtgcaggagcaaggtgagatgacaggag

SEQ ID NO: 228 - DNA, 58 bp, Artificial Sequence
  Synthetic Polynucleotide
tctatagtgtcacctaaatcgtatgcgactgcacaggagcaaggtgagatgacaggag

SEQ ID NO: 229 - DNA, 58 bp, Artificial Sequence
  Synthetic Polynucleotide
tctatagtgtcacctaaatcgtatgcggtcaatcaggagcaaggtgagatgacaggag

SEQ ID NO: 230 - DNA, 58 bp, Artificial Sequence
  Synthetic Polynucleotide
tctatagtgtcacctaaatcgtatggctgctaccaggagcaaggtgagatgacaggag

SEQ ID NO: 231 - DNA, 58 bp, Artificial Sequence
  Synthetic Polynucleotide
tctatagtgtcacctaaatcgtatggcagtctacaggagcaaggtgagatgacaggag

SEQ ID NO: 232 - DNA, 58 bp, Artificial Sequence
  Synthetic Polynucleotide
tctatagtgtcacctaaatcgtatgtggaccaccaggagcaaggtgagatgacaggag

SEQ ID NO: 233 - DNA, 58 bp, Artificial Sequence
  Synthetic Polynucleotide
tctatagtgtcacctaaatcgtatggtcacatccaggagcaaggtgagatgacaggag

SEQ ID NO: 234 - DNA, 58 bp, Artificial Sequence
  Synthetic Polynucleotide
tctatagtgtcacctaaatcgtatggttgctgacaggagcaaggtgagatgacaggag

SEQ ID NO: 235 - DNA, 58 bp, Artificial Sequence
  Synthetic Polynucleotide
tctatagtgtcacctaaatcgtatggagttagccaggagcaaggtgagatgacaggag

SEQ ID NO: 236 - DNA, 58 bp, Artificial Sequence
  Synthetic Polynucleotide
tctatagtgtcacctaaatcgtatgacgatcatcaggagcaaggtgagatgacaggag

SEQ ID NO: 237 - DNA, 58 bp, Artificial Sequence
  Synthetic Polynucleotide
tctatagtgtcacctaaatcgtatgcgtcgtctcaggagcaaggtgagatgacaggag

SEQ ID NO: 238 - DNA, 58 bp, Artificial Sequence
  Synthetic Polynucleotide
tctatagtgtcacctaaatcgtatggacatgcgcaggagcaaggtgagatgacaggag

SEQ ID NO: 239 - DNA, 58 bp, Artificial Sequence
  Synthetic Polynucleotide
tctatagtgtcacctaaatcgtatggtgccatacaggagcaaggtgagatgacaggag

SEQ ID NO: 240 - DNA, 58 bp, Artificial Sequence
  Synthetic Polynucleotide
tctatagtgtcacctaaatcgtatgcgctaggacaggagcaaggtgagatgacaggag

SEQ ID NO: 241 - DNA, 58 bp, Artificial Sequence
  Synthetic Polynucleotide
tctatagtgtcacctaaatcgtatgcgtaggtacaggagcaaggtgagatgacaggag

SEQ ID NO: 242 - DNA, 58 bp, Artificial Sequence
  Synthetic Polynucleotide
tctatagtgtcacctaaatcgtatgagctagcgcaggagcaaggtgagatgacaggag

SEQ ID NO: 243 - DNA, 58 bp, Artificial Sequence
  Synthetic Polynucleotide
tctatagtgtcacctaaatcgtatgtcctgtgccaggagcaaggtgagatgacaggag

SEQ ID NO: 244 - DNA, 58 bp, Artificial Sequence
  Synthetic Polynucleotide
tctatagtgtcacctaaatcgtatggtaatctgcaggagcaaggtgagatgacaggag

SEQ ID NO: 245 - DNA, 58 bp, Artificial Sequence
  Synthetic Polynucleotide
tctatagtgtcacctaaatcgtatgaacgtaggcaggagcaaggtgagatgacaggag

SEQ ID NO: 246 - DNA, 58 bp, Artificial Sequence
  Synthetic Polynucleotide
tctatagtgtcacctaaatcgtatgttcctgttcaggagcaaggtgagatgacaggag

SEQ ID NO: 247 - DNA, 58 bp, Artificial Sequence
  Synthetic Polynucleotide
tctatagtgtcacctaaatcgtatgtgtccagtcaggagcaaggtgagatgacaggag

SEQ ID NO: 248 - DNA, 58 bp, Artificial Sequence
  Synthetic Polynucleotide
tctatagtgtcacctaaatcgtatgacaaggcacaggagcaaggtgagatgacaggag

SEQ ID NO: 249 - DNA, 58 bp, Artificial Sequence
  Synthetic Polynucleotide
tctatagtgtcacctaaatcgtatgccttgacccaggagcaaggtgagatgacaggag

SEQ ID NO: 250 - DNA, 58 bp, Artificial Sequence
  Synthetic Polynucleotide
tctatagtgtcacctaaatcgtatgcgcttgtgcaggagcaaggtgagatgacaggag

SEQ ID NO: 251 - DNA, 58 bp, Artificial Sequence
  Synthetic Polynucleotide
tctatagtgtcacctaaatcgtatgtccaagcgcaggagcaaggtgagatgacaggag

SEQ ID NO: 252 - DNA, 58 bp, Artificial Sequence
  Synthetic Polynucleotide
tctatagtgtcacctaaatcgtatgctagtgaccaggagcaaggtgagatgacaggag

SEQ ID NO: 253 - DNA, 58 bp, Artificial Sequence
  Synthetic Polynucleotide
tctatagtgtcacctaaatcgtatgagaaccgtcaggagcaaggtgagatgacaggag

SEQ ID NO: 254 - DNA, 58 bp, Artificial Sequence
  Synthetic Polynucleotide
tctatagtgtcacctaaatcgtatgtaattgcacaggagcaaggtgagatgacaggag

SEQ ID NO: 255 - DNA, 58 bp, Artificial Sequence
  Synthetic Polynucleotide
tctatagtgtcacctaaatcgtatgctagtacacaggagcaaggtgagatgacaggag

SEQ ID NO: 256 - DNA, 58 bp, Artificial Sequence
  Synthetic Polynucleotide
tctatagtgtcacctaaatcgtatggctatatccaggagcaaggtgagatgacaggag

SEQ ID NO: 257 - DNA, 58 bp, Artificial Sequence
  Synthetic Polynucleotide
tctatagtgtcacctaaatcgtatgcaatcggccaggagcaaggtgagatgacaggag

SEQ ID NO: 258 - DNA, 58 bp, Artificial Sequence
  Synthetic Polynucleotide
tctatagtgtcacctaaatcgtatgcgatatcacaggagcaaggtgagatgacaggag

SEQ ID NO: 259 - DNA, 58 bp, Artificial Sequence
  Synthetic Polynucleotide
tctatagtgtcacctaaatcgtatgcagtcaggcaggagcaaggtgagatgacaggag

SEQ ID NO: 260 - DNA, 58 bp, Artificial Sequence
  Synthetic Polynucleotide
tctatagtgtcacctaaatcgtatggtaataatcaggagcaaggtgagatgacaggag

SEQ ID NO: 261 - DNA, 58 bp, Artificial Sequence
  Synthetic Polynucleotide
tctatagtgtcacctaaatcgtatgggagagatcaggagcaaggtgagatgacaggag

SEQ ID NO: 262 - DNA, 58 bp, Artificial Sequence
  Synthetic Polynucleotide
tctatagtgtcacctaaatcgtatgctctcatacaggagcaaggtgagatgacaggag

SEQ ID NO: 263 - DNA, 58 bp, Artificial Sequence
  Synthetic Polynucleotide
tctatagtgtcacctaaatcgtatgcagcgactcaggagcaaggtgagatgacaggag

SEQ ID NO: 264 - DNA, 58 bp, Artificial Sequence
  Synthetic Polynucleotide
tctatagtgtcacctaaatcgtatgggccaaggcaggagcaaggtgagatgacaggag

SEQ ID NO: 265 - DNA, 58 bp, Artificial Sequence
  Synthetic Polynucleotide
tctatagtgtcacctaaatcgtatggcatatgccaggagcaaggtgagatgacaggag

SEQ ID NO: 266 - DNA, 58 bp, Artificial Sequence
  Synthetic Polynucleotide
tctatagtgtcacctaaatcgtatgactaggatcaggagcaaggtgagatgacaggag

SEQ ID NO: 267 - DNA, 58 bp, Artificial Sequence
  Synthetic Polynucleotide
tctatagtgtcacctaaatcgtatgccttacctcaggagcaaggtgagatgacaggag

SEQ ID NO: 268 - DNA, 58 bp, Artificial Sequence
  Synthetic Polynucleotide
tctatagtgtcacctaaatcgtatgtgttgacgcaggagcaaggtgagatgacaggag

SEQ ID NO: 269 - DNA, 58 bp, Artificial Sequence
  Synthetic Polynucleotide
tctatagtgtcacctaaatcgtatgtacagttacaggagcaaggtgagatgacaggag

SEQ ID NO: 270 - DNA, 58 bp, Artificial Sequence
  Synthetic Polynucleotide
tctatagtgtcacctaaatcgtatgttgttacgcaggagcaaggtgagatgacaggag

SEQ ID NO: 271 - DNA, 58 bp, Artificial Sequence
  Synthetic Polynucleotide
tctatagtgtcacctaaatcgtatgtcgtgttgcaggagcaaggtgagatgacaggag

SEQ ID NO: 272 - DNA, 58 bp, Artificial Sequence
  Synthetic Polynucleotide
tctatagtgtcacctaaatcgtatgagtcaatgcaggagcaaggtgagatgacaggag

SEQ ID NO: 273 - DNA, 58 bp, Artificial Sequence
  Synthetic Polynucleotide
tctatagtgtcacctaaatcgtatgtctgtagacaggagcaaggtgagatgacaggag

SEQ ID NO: 274 - DNA, 58 bp, Artificial Sequence
  Synthetic Polynucleotide
tctatagtgtcacctaaatcgtatggacaacgacaggagcaaggtgagatgacaggag

SEQ ID NO: 275 - DNA, 58 bp, Artificial Sequence
  Synthetic Polynucleotide
tctatagtgtcacctaaatcgtatgccatggctcaggagcaaggtgagatgacaggag

SEQ ID NO: 276 - DNA, 58 bp, Artificial Sequence
  Synthetic Polynucleotide
tctatagtgtcacctaaatcgtatgtgactctgcaggagcaaggtgagatgacaggag

SEQ ID NO: 277 - DNA, 58 bp, Artificial Sequence
  Synthetic Polynucleotide
tctatagtgtcacctaaatcgtatgaacgaggccaggagcaaggtgagatgacaggag

SEQ ID NO: 278 - DNA, 58 bp, Artificial Sequence
  Synthetic Polynucleotide
tctatagtgtcacctaaatcgtatgcagaaggtcaggagcaaggtgagatgacaggag

SEQ ID NO: 279 - DNA, 58 bp, Artificial Sequence
  Synthetic Polynucleotide
tctatagtgtcacctaaatcgtatgtgaagtcacaggagcaaggtgagatgacaggag

SEQ ID NO: 280 - DNA, 58 bp, Artificial Sequence
  Synthetic Polynucleotide
tctatagtgtcacctaaatcgtatgatgttcctcaggagcaaggtgagatgacaggag

SEQ ID NO: 281 - DNA, 58 bp, Artificial Sequence
  Synthetic Polynucleotide
tctatagtgtcacctaaatcgtatgaagtggctcaggagcaaggtgagatgacaggag

SEQ ID NO: 282 - DNA, 58 bp, Artificial Sequence
  Synthetic Polynucleotide
tctatagtgtcacctaaatcgtatgggtacaatcaggagcaaggtgagatgacaggag

SEQ ID NO: 283 - DNA, 58 bp, Artificial Sequence
  Synthetic Polynucleotide
tctatagtgtcacctaaatcgtatgacaagtgccaggagcaaggtgagatgacaggag

SEQ ID NO: 284 - DNA, 58 bp, Artificial Sequence
  Synthetic Polynucleotide
tctatagtgtcacctaaatcgtatgtcacggtgcaggagcaaggtgagatgacaggag

SEQ ID NO: 285 - DNA, 58 bp, Artificial Sequence
  Synthetic Polynucleotide
tctatagtgtcacctaaatcgtatgttgcgttacaggagcaaggtgagatgacaggag

SEQ ID NO: 286 - DNA, 58 bp, Artificial Sequence
  Synthetic Polynucleotide
tctatagtgtcacctaaatcgtatgttgtagcccaggagcaaggtgagatgacaggag

SEQ ID NO: 287 - DNA, 58 bp, Artificial Sequence
  Synthetic Polynucleotide
tctatagtgtcacctaaatcgtatgtcaccggacaggagcaaggtgagatgacaggag

SEQ ID NO: 288 - DNA, 58 bp, Artificial Sequence
  Synthetic Polynucleotide
tctatagtgtcacctaaatcgtatgcgcgcaagcaggagcaaggtgagatgacaggag

SEQ ID NO: 289 - DNA, 54 bp, Artificial Sequence
  Synthetic Polynucleotide
tctctatgggcagtcggtgattagatcgcctcctgtcatctcaccttgctccac

SEQ ID NO: 290 - DNA, 54 bp, Artificial Sequence
  Synthetic Polynucleotide
tctctatgggcagtcggtgatctctctatctcctgtcatctcaccttgctccac

SEQ ID NO: 291 - DNA, 54 bp, Artificial Sequence
  Synthetic Polynucleotide
tctctatgggcagtcggtgattatcctctctcctgtcatctcaccttgctccac

SEQ ID NO: 292 - DNA, 54 bp, Artificial Sequence
  Synthetic Polynucleotide
tctctatgggcagtcggtgatagagtagactcctgtcatctcaccttgctccac

SEQ ID NO: 293 - DNA, 54 bp, Artificial Sequence
  Synthetic Polynucleotide
tctctatgggcagtcggtgatgtaaggagctcctgtcatctcaccttgctccac

SEQ ID NO: 294 - DNA, 54 bp, Artificial Sequence
  Synthetic Polynucleotide
tctctatgggcagtcggtgatactgcatactcctgtcatctcaccttgctccac

SEQ ID NO: 295 - DNA, 54 bp, Artificial Sequence
  Synthetic Polynucleotide
tctctatgggcagtcggtgataaggagtactcctgtcatctcaccttgctccac

SEQ ID NO: 296 - DNA, 54 bp, Artificial Sequence
  Synthetic Polynucleotide
tctctatgggcagtcggtgatctaagcctctcctgtcatctcaccttgctccac

SEQ ID NO: 297 - DNA, 54 bp, Artificial Sequence
  Synthetic Polynucleotide
tctctatgggcagtcggtgatgacattgtctcctgtcatctcaccttgctccac

SEQ ID NO: 298 - DNA, 54 bp, Artificial Sequence
  Synthetic Polynucleotide
tctctatgggcagtcggtgatactgatggctcctgtcatctcaccttgctccac

SEQ ID NO: 299 - DNA, 54 bp, Artificial Sequence
  Synthetic Polynucleotide
tctctatgggcagtcggtgatgtacctagctcctgtcatctcaccttgctccac

SEQ ID NO: 300 - DNA, 54 bp, Artificial Sequence
  Synthetic Polynucleotide
tctctatgggcagtcggtgatcagagctactcctgtcatctcaccttgctccac

SEQ ID NO: 301 - DNA, 54 bp, Artificial Sequence
  Synthetic Polynucleotide
tctctatgggcagtcggtgatcatagtgactcctgtcatctcaccttgctccac

SEQ ID NO: 302 - DNA, 54 bp, Artificial Sequence
  Synthetic Polynucleotide
tctctatgggcagtcggtgattacctagtctcctgtcatctcaccttgctccac

SEQ ID NO: 303 - DNA, 54 bp, Artificial Sequence
  Synthetic Polynucleotide
tctctatgggcagtcggtgatcgcgatatctcctgtcatctcaccttgctccac

SEQ ID NO: 304 - DNA, 54 bp, Artificial Sequence
  Synthetic Polynucleotide
tctctatgggcagtcggtgattggattgtctcctgtcatctcaccttgctccac

SEQ ID NO: 305 - DNA, 54 bp, Artificial Sequence
  Synthetic Polynucleotide
tctctatgggcagtcggtgatggacttccctcctgtcatctcaccttgctccac

SEQ ID NO: 306 - DNA, 54 bp, Artificial Sequence
  Synthetic Polynucleotide
tctctatgggcagtcggtgatggtatggcctcctgtcatctcaccttgctccac

SEQ ID NO: 307 - DNA, 54 bp, Artificial Sequence
  Synthetic Polynucleotide
tctctatgggcagtcggtgatcaatacgactcctgtcatctcaccttgctccac

SEQ ID NO: 308 - DNA, 54 bp, Artificial Sequence
  Synthetic Polynucleotide
tctctatgggcagtcggtgatacaggtagctcctgtcatctcaccttgctccac

SEQ ID NO: 309 - DNA, 54 bp, Artificial Sequence
  Synthetic Polynucleotide
tctctatgggcagtcggtgattggagaggctcctgtcatctcaccttgctccac

SEQ ID NO: 310 - DNA, 54 bp, Artificial Sequence
  Synthetic Polynucleotide
tctctatgggcagtcggtgatttacgtggctcctgtcatctcaccttgctccac

SEQ ID NO: 311 - DNA, 54 bp, Artificial Sequence
  Synthetic Polynucleotide
tctctatgggcagtcggtgatatagccgactcctgtcatctcaccttgctccac

SEQ ID NO: 312 - DNA, 54 bp, Artificial Sequence
  Synthetic Polynucleotide
tctctatgggcagtcggtgatgagtatctctcctgtcatctcaccttgctccac

SEQ ID NO: 313 - DNA, 54 bp, Artificial Sequence
  Synthetic Polynucleotide
tctctatgggcagtcggtgatgtcgtgtactcctgtcatctcaccttgctccac

SEQ ID NO: 314 - DNA, 54 bp, Artificial Sequence
  Synthetic Polynucleotide
tctctatgggcagtcggtgattatccaagctcctgtcatctcaccttgctccac

SEQ ID NO: 315 - DNA, 54 bp, Artificial Sequence
  Synthetic Polynucleotide
tctctatgggcagtcggtgatagtcgctactcctgtcatctcaccttgctccac

SEQ ID NO: 316 - DNA, 54 bp, Artificial Sequence
  Synthetic Polynucleotide
tctctatgggcagtcggtgatgacaggttctcctgtcatctcaccttgctccac

SEQ ID NO: 317 - DNA, 54 bp, Artificial Sequence
  Synthetic Polynucleotide
tctctatgggcagtcggtgatagattgacctcctgtcatctcaccttgctccac

SEQ ID NO: 318 - DNA, 54 bp, Artificial Sequence
  Synthetic Polynucleotide
tctctatgggcagtcggtgattatcaccgctcctgtcatctcaccttgctccac

SEQ ID NO: 319 - DNA, 54 bp, Artificial Sequence
  Synthetic Polynucleotide
tctctatgggcagtcggtgatataggctgctcctgtcatctcaccttgctccac

SEQ ID NO: 320 - DNA, 54 bp, Artificial Sequence
  Synthetic Polynucleotide
tctctatgggcagtcggtgatagtggcacctcctgtcatctcaccttgctccac

SEQ ID NO: 321 - DNA, 54 bp, Artificial Sequence
  Synthetic Polynucleotide
tctctatgggcagtcggtgatactcatctctcctgtcatctcaccttgctccac

SEQ ID NO: 322 - DNA, 54 bp, Artificial Sequence
  Synthetic Polynucleotide
tctctatgggcagtcggtgatgcagccatctcctgtcatctcaccttgctccac

SEQ ID NO: 323 - DNA, 54 bp, Artificial Sequence
  Synthetic Polynucleotide
tctctatgggcagtcggtgatttgcagtgctcctgtcatctcaccttgctccac

SEQ ID NO: 324 - DNA, 54 bp, Artificial Sequence
  Synthetic Polynucleotide
tctctatgggcagtcggtgatcgactgcactcctgtcatctcaccttgctccac

SEQ ID NO: 325 - DNA, 54 bp, Artificial Sequence
  Synthetic Polynucleotide
tctctatgggcagtcggtgatcggtcaatctcctgtcatctcaccttgctccac

SEQ ID NO: 326 - DNA, 54 bp, Artificial Sequence
  Synthetic Polynucleotide
tctctatgggcagtcggtgatgctgctacctcctgtcatctcaccttgctccac

SEQ ID NO: 327 - DNA, 54 bp, Artificial Sequence
  Synthetic Polynucleotide
tctctatgggcagtcggtgatgcagtctactcctgtcatctcaccttgctccac

SEQ ID NO: 328 - DNA, 54 bp, Artificial Sequence
  Synthetic Polynucleotide
tctctatgggcagtcggtgattggaccacctcctgtcatctcaccttgctccac

SEQ ID NO: 329 - DNA, 54 bp, Artificial Sequence
  Synthetic Polynucleotide
tctctatgggcagtcggtgatgtcacatcctcctgtcatctcaccttgctccac

SEQ ID NO: 330 - DNA, 54 bp, Artificial Sequence
  Synthetic Polynucleotide
tctctatgggcagtcggtgatgttgctgactcctgtcatctcaccttgctccac

SEQ ID NO: 331 - DNA, 54 bp, Artificial Sequence
  Synthetic Polynucleotide
tctctatgggcagtcggtgatgagttagcctcctgtcatctcaccttgctccac

SEQ ID NO: 332 - DNA, 54 bp, Artificial Sequence
  Synthetic Polynucleotide
tctctatgggcagtcggtgatacgatcatctcctgtcatctcaccttgctccac

SEQ ID NO: 333 - DNA, 54 bp, Artificial Sequence
  Synthetic Polynucleotide
tctctatgggcagtcggtgatcgtcgtctctcctgtcatctcaccttgctccac

SEQ ID NO: 334 - DNA, 54 bp, Artificial Sequence
  Synthetic Polynucleotide
tctctatgggcagtcggtgatgacatgcgctcctgtcatctcaccttgctccac

SEQ ID NO: 335 - DNA, 54 bp, Artificial Sequence
  Synthetic Polynucleotide
tctctatgggcagtcggtgatgtgccatactcctgtcatctcaccttgctccac

SEQ ID NO: 336 - DNA, 54 bp, Artificial Sequence
  Synthetic Polynucleotide
tctctatgggcagtcggtgatcgctaggactcctgtcatctcaccttgctccac

SEQ ID NO: 337 - DNA, 54 bp, Artificial Sequence
  Synthetic Polynucleotide
tctctatgggcagtcggtgatcgtaggtactcctgtcatctcaccttgctccac

SEQ ID NO: 338 - DNA, 54 bp, Artificial Sequence
  Synthetic Polynucleotide
tctctatgggcagtcggtgatagctagcgctcctgtcatctcaccttgctccac

SEQ ID NO: 339 - DNA, 54 bp, Artificial Sequence
  Synthetic Polynucleotide
tctctatgggcagtcggtgattcctgtgcctcctgtcatctcaccttgctccac

SEQ ID NO: 340 - DNA, 54 bp, Artificial Sequence
  Synthetic Polynucleotide
tctctatgggcagtcggtgatgtaatctgctcctgtcatctcaccttgctccac

SEQ ID NO: 341 - DNA, 54 bp, Artificial Sequence
  Synthetic Polynucleotide
tctctatgggcagtcggtgataacgtaggctcctgtcatctcaccttgctccac

SEQ ID NO: 342 - DNA, 54 bp, Artificial Sequence
  Synthetic Polynucleotide
tctctatgggcagtcggtgatttcctgttctcctgtcatctcaccttgctccac

SEQ ID NO: 343 - DNA, 54 bp, Artificial Sequence
  Synthetic Polynucleotide
tctctatgggcagtcggtgattgtccagtctcctgtcatctcaccttgctccac

SEQ ID NO: 344 - DNA, 54 bp, Artificial Sequence
  Synthetic Polynucleotide
tctctatgggcagtcggtgatacaaggcactcctgtcatctcaccttgctccac

SEQ ID NO: 345 - DNA, 54 bp, Artificial Sequence
  Synthetic Polynucleotide
tctctatgggcagtcggtgatccttgaccctcctgtcatctcaccttgctccac

SEQ ID NO: 346 - DNA, 54 bp, Artificial Sequence
  Synthetic Polynucleotide
tctctatgggcagtcggtgatcgcttgtgctcctgtcatctcaccttgctccac

SEQ ID NO: 347 - DNA, 54 bp, Artificial Sequence
  Synthetic Polynucleotide
tctctatgggcagtcggtgattccaagcgctcctgtcatctcaccttgctccac

SEQ ID NO: 348 - DNA, 54 bp, Artificial Sequence
  Synthetic Polynucleotide
tctctatgggcagtcggtgatctagtgacctcctgtcatctcaccttgctccac

SEQ ID NO: 349 - DNA, 54 bp, Artificial Sequence
  Synthetic Polynucleotide
tctctatgggcagtcggtgatagaaccgtctcctgtcatctcaccttgctccac

SEQ ID NO: 350 - DNA, 54 bp, Artificial Sequence
  Synthetic Polynucleotide
tctctatgggcagtcggtgattaattgcactcctgtcatctcaccttgctccac

SEQ ID NO: 351 - DNA, 54 bp, Artificial Sequence
  Synthetic Polynucleotide
tctctatgggcagtcggtgatctagtacactcctgtcatctcaccttgctccac

SEQ ID NO: 352 - DNA, 54 bp, Artificial Sequence
  Synthetic Polynucleotide
tctctatgggcagtcggtgatgctatatcctcctgtcatctcaccttgctccac

SEQ ID NO: 353 - DNA, 54 bp, Artificial Sequence
  Synthetic Polynucleotide
tctctatgggcagtcggtgatcaatcggcctcctgtcatctcaccttgctccac

SEQ ID NO: 354 - DNA, 54 bp, Artificial Sequence
  Synthetic Polynucleotide
tctctatgggcagtcggtgatcgatatcactcctgtcatctcaccttgctccac

SEQ ID NO: 355 - DNA, 54 bp, Artificial Sequence
  Synthetic Polynucleotide
tctctatgggcagtcggtgatcagtcaggctcctgtcatctcaccttgctccac

SEQ ID NO: 356 - DNA, 54 bp, Artificial Sequence
  Synthetic Polynucleotide
tctctatgggcagtcggtgatgtaataatctcctgtcatctcaccttgctccac

SEQ ID NO: 357 - DNA, 54 bp, Artificial Sequence
  Synthetic Polynucleotide
tctctatgggcagtcggtgatggagagatctcctgtcatctcaccttgctccac

SEQ ID NO: 358 - DNA, 54 bp, Artificial Sequence
  Synthetic Polynucleotide
tctctatgggcagtcggtgatctctcatactcctgtcatctcaccttgctccac

SEQ ID NO: 359 - DNA, 54 bp, Artificial Sequence
  Synthetic Polynucleotide
tctctatgggcagtcggtgatcagcgactctcctgtcatctcaccttgctccac

SEQ ID NO: 360 - DNA, 54 bp, Artificial Sequence
  Synthetic Polynucleotide
tctctatgggcagtcggtgatggccaaggctcctgtcatctcaccttgctccac

SEQ ID NO: 361 - DNA, 54 bp, Artificial Sequence
  Synthetic Polynucleotide
tctctatgggcagtcggtgatgcatatgcctcctgtcatctcaccttgctccac

SEQ ID NO: 362 - DNA, 54 bp, Artificial Sequence
  Synthetic Polynucleotide
tctctatgggcagtcggtgatactaggatctcctgtcatctcaccttgctccac

SEQ ID NO: 363 - DNA, 54 bp, Artificial Sequence
  Synthetic Polynucleotide
tctctatgggcagtcggtgatccttacctctcctgtcatctcaccttgctccac

SEQ ID NO: 364 - DNA, 54 bp, Artificial Sequence
  Synthetic Polynucleotide
tctctatgggcagtcggtgattgttgacgctcctgtcatctcaccttgctccac

SEQ ID NO: 365 - DNA, 54 bp, Artificial Sequence
  Synthetic Polynucleotide
tctctatgggcagtcggtgattacagttactcctgtcatctcaccttgctccac

SEQ ID NO: 366 - DNA, 54 bp, Artificial Sequence
  Synthetic Polynucleotide
tctctatgggcagtcggtgatttgttacgctcctgtcatctcaccttgctccac

SEQ ID NO: 367 - DNA, 54 bp, Artificial Sequence
  Synthetic Polynucleotide
tctctatgggcagtcggtgattcgtgttgctcctgtcatctcaccttgctccac

SEQ ID NO: 368 - DNA, 54 bp, Artificial Sequence
  Synthetic Polynucleotide
tctctatgggcagtcggtgatagtcaatgctcctgtcatctcaccttgctccac

SEQ ID NO: 369 - DNA, 54 bp, Artificial Sequence
  Synthetic Polynucleotide
tctctatgggcagtcggtgattctgtagactcctgtcatctcaccttgctccac

SEQ ID NO: 370 - DNA, 54 bp, Artificial Sequence
  Synthetic Polynucleotide
tctctatgggcagtcggtgatgacaacgactcctgtcatctcaccttgctccac

SEQ ID NO: 371 - DNA, 54 bp, Artificial Sequence
  Synthetic Polynucleotide
tctctatgggcagtcggtgatccatggctctcctgtcatctcaccttgctccac

SEQ ID NO: 372 - DNA, 54 bp, Artificial Sequence
  Synthetic Polynucleotide
tctctatgggcagtcggtgattgactctgctcctgtcatctcaccttgctccac

SEQ ID NO: 373 - DNA, 54 bp, Artificial Sequence
  Synthetic Polynucleotide
tctctatgggcagtcggtgataacgaggcctcctgtcatctcaccttgctccac

SEQ ID NO: 374 - DNA, 54 bp, Artificial Sequence
  Synthetic Polynucleotide
tctctatgggcagtcggtgatcagaaggtctcctgtcatctcaccttgctccac

SEQ ID NO: 375 - DNA, 54 bp, Artificial Sequence
  Synthetic Polynucleotide
tctctatgggcagtcggtgattgaagtcactcctgtcatctcaccttgctccac

SEQ ID NO: 376 - DNA, 54 bp, Artificial Sequence
  Synthetic Polynucleotide
tctctatgggcagtcggtgatatgttcctctcctgtcatctcaccttgctccac

SEQ ID NO: 377 - DNA, 54 bp, Artificial Sequence
  Synthetic Polynucleotide
tctctatgggcagtcggtgataagtggctctcctgtcatctcaccttgctccac

SEQ ID NO: 378 - DNA, 54 bp, Artificial Sequence
  Synthetic Polynucleotide
tctctatgggcagtcggtgatggtacaatctcctgtcatctcaccttgctccac

SEQ ID NO: 379 - DNA, 54 bp, Artificial Sequence
  Synthetic Polynucleotide
tctctatgggcagtcggtgatacaagtgcctcctgtcatctcaccttgctccac

SEQ ID NO: 380 - DNA, 54 bp, Artificial Sequence
  Synthetic Polynucleotide
tctctatgggcagtcggtgattcacggtgctcctgtcatctcaccttgctccac

SEQ ID NO: 381 - DNA, 54 bp, Artificial Sequence
  Synthetic Polynucleotide
tctctatgggcagtcggtgatttgcgttactcctgtcatctcaccttgctccac

SEQ ID NO: 382 - DNA, 54 bp, Artificial Sequence
  Synthetic Polynucleotide
tctctatgggcagtcggtgatttgtagccctcctgtcatctcaccttgctccac

SEQ ID NO: 383 - DNA, 54 bp, Artificial Sequence
  Synthetic Polynucleotide
tctctatgggcagtcggtgattcaccggactcctgtcatctcaccttgctccac

SEQ ID NO: 384 - DNA, 54 bp, Artificial Sequence
  Synthetic Polynucleotide
tctctatgggcagtcggtgatcgcgcaagctcctgtcatctcaccttgctccac

SEQ ID NO: 385 - DNA, 65 bp, Artificial Sequence
  Synthetic Polynucleotide
tcaccgactgcccatagagaggactccagtcactagatcgcatctcgtatgccgtcttctgcttg

SEQ ID NO: 386 - DNA, 65 bp, Artificial Sequence
  Synthetic Polynucleotide
tcaccgactgcccatagagaggactccagtcacctctctatatctcgtatgccgtcttctgcttg

SEQ ID NO: 387 - DNA, 65 bp, Artificial Sequence
  Synthetic Polynucleotide
tcaccgactgcccatagagaggactccagtcactatcctctatctcgtatgccgtcttctgcttg

SEQ ID NO: 388 - DNA, 65 bp, Artificial Sequence
  Synthetic Polynucleotide
tcaccgactgcccatagagaggactccagtcacagagtagaatctcgtatgccgtcttctgcttg

SEQ ID NO: 389 - DNA, 65 bp, Artificial Sequence
  Synthetic Polynucleotide
tcaccgactgcccatagagaggactccagtcacgtaaggagatctcgtatgccgtcttctgcttg

SEQ ID NO: 390 - DNA, 65 bp, Artificial Sequence
  Synthetic Polynucleotide
tcaccgactgcccatagagaggactccagtcacactgcataatctcgtatgccgtcttctgcttg

SEQ ID NO: 391 - DNA, 65 bp, Artificial Sequence
  Synthetic Polynucleotide
tcaccgactgcccatagagaggactccagtcacaaggagtaatctcgtatgccgtcttctgcttg

SEQ ID NO: 392 - DNA, 65 bp, Artificial Sequence
  Synthetic Polynucleotide
tcaccgactgcccatagagaggactccagtcacctaagcctatctcgtatgccgtcttctgcttg

SEQ ID NO: 393 - DNA, 65 bp, Artificial Sequence
  Synthetic Polynucleotide
tcaccgactgcccatagagaggactccagtcacgacattgtatctcgtatgccgtcttctgcttg

SEQ ID NO: 394 - DNA, 65 bp, Artificial Sequence
  Synthetic Polynucleotide
tcaccgactgcccatagagaggactccagtcacactgatggatctcgtatgccgtcttctgcttg

SEQ ID NO: 395 - DNA, 65 bp, Artificial Sequence
  Synthetic Polynucleotide
tcaccgactgcccatagagaggactccagtcacgtacctagatctcgtatgccgtcttctgcttg

SEQ ID NO: 396 - DNA, 65 bp, Artificial Sequence
  Synthetic Polynucleotide
tcaccgactgcccatagagaggactccagtcaccagagctaatctcgtatgccgtcttctgcttg

SEQ ID NO: 397 - DNA, 65 bp, Artificial Sequence
  Synthetic Polynucleotide
tcaccgactgcccatagagaggactccagtcaccatagtgaatctcgtatgccgtcttctgcttg

SEQ ID NO: 398 - DNA, 65 bp, Artificial Sequence
  Synthetic Polynucleotide
tcaccgactgcccatagagaggactccagtcactacctagtatctcgtatgccgtcttctgcttg

SEQ ID NO: 399 - DNA, 65 bp, Artificial Sequence
  Synthetic Polynucleotide
tcaccgactgcccatagagaggactccagtcaccgcgatatatctcgtatgccgtcttctgcttg

SEQ ID NO: 400 - DNA, 65 bp, Artificial Sequence
  Synthetic Polynucleotide
tcaccgactgcccatagagaggactccagtcactggattgtatctcgtatgccgtcttctgcttg

SEQ ID NO: 401 - DNA, 65 bp, Artificial Sequence
  Synthetic Polynucleotide
tcaccgactgcccatagagaggactccagtcacggacttccatctcgtatgccgtcttctgcttg

SEQ ID NO: 402 - DNA, 65 bp, Artificial Sequence
  Synthetic Polynucleotide
tcaccgactgcccatagagaggactccagtcacggtatggcatctcgtatgccgtcttctgcttg

SEQ ID NO: 403 - DNA, 65 bp, Artificial Sequence
  Synthetic Polynucleotide
tcaccgactgcccatagagaggactccagtcaccaatacgaatctcgtatgccgtcttctgcttg

SEQ ID NO: 404 - DNA, 65 bp, Artificial Sequence
  Synthetic Polynucleotide
tcaccgactgcccatagagaggactccagtcacacaggtagatctcgtatgccgtcttctgcttg

SEQ ID NO: 405 - DNA, 65 bp, Artificial Sequence
  Synthetic Polynucleotide
tcaccgactgcccatagagaggactccagtcactggagaggatctcgtatgccgtcttctgcttg

SEQ ID NO: 406 - DNA, 65 bp, Artificial Sequence
  Synthetic Polynucleotide
tcaccgactgcccatagagaggactccagtcacttacgtggatctcgtatgccgtcttctgcttg

SEQ ID NO: 407 - DNA, 65 bp, Artificial Sequence
  Synthetic Polynucleotide
tcaccgactgcccatagagaggactccagtcacatagccgaatctcgtatgccgtcttctgcttg

SEQ ID NO: 408 - DNA, 65 bp, Artificial Sequence
  Synthetic Polynucleotide
tcaccgactgcccatagagaggactccagtcacgagtatctatctcgtatgccgtcttctgcttg

SEQ ID NO: 409 - DNA, 65 bp, Artificial Sequence
  Synthetic Polynucleotide
tcaccgactgcccatagagaggactccagtcacgtcgtgtaatctcgtatgccgtcttctgcttg

SEQ ID NO: 410 - DNA, 65 bp, Artificial Sequence
  Synthetic Polynucleotide
tcaccgactgcccatagagaggactccagtcactatccaagatctcgtatgccgtcttctgcttg

SEQ ID NO: 411 - DNA, 65 bp, Artificial Sequence
  Synthetic Polynucleotide
tcaccgactgcccatagagaggactccagtcacagtcgctaatctcgtatgccgtcttctgcttg

SEQ ID NO: 412 - DNA, 65 bp, Artificial Sequence
  Synthetic Polynucleotide
tcaccgactgcccatagagaggactccagtcacgacaggttatctcgtatgccgtcttctgcttg

SEQ ID NO: 413 - DNA, 65 bp, Artificial Sequence
  Synthetic Polynucleotide
tcaccgactgcccatagagaggactccagtcacagattgacatctcgtatgccgtcttctgcttg

SEQ ID NO: 414 - DNA, 65 bp, Artificial Sequence
  Synthetic Polynucleotide
tcaccgactgcccatagagaggactccagtcactatcaccgatctcgtatgccgtcttctgcttg

SEQ ID NO: 415 - DNA, 65 bp, Artificial Sequence
  Synthetic Polynucleotide
tcaccgactgcccatagagaggactccagtcacataggctgatctcgtatgccgtcttctgcttg

SEQ ID NO: 416 - DNA, 65 bp, Artificial Sequence
  Synthetic Polynucleotide
tcaccgactgcccatagagaggactccagtcacagtggcacatctcgtatgccgtcttctgcttg

SEQ ID NO: 417 - DNA, 65 bp, Artificial Sequence
  Synthetic Polynucleotide
tcaccgactgcccatagagaggactccagtcacactcatctatctcgtatgccgtcttctgcttg

SEQ ID NO: 418 - DNA, 65 bp, Artificial Sequence
  Synthetic Polynucleotide
tcaccgactgcccatagagaggactccagtcacgcagccatatctcgtatgccgtcttctgcttg

SEQ ID NO: 419 - DNA, 65 bp, Artificial Sequence
  Synthetic Polynucleotide
tcaccgactgcccatagagaggactccagtcacttgcagtgatctcgtatgccgtcttctgcttg

SEQ ID NO: 420 - DNA, 65 bp, Artificial Sequence
  Synthetic Polynucleotide
tcaccgactgcccatagagaggactccagtcaccgactgcaatctcgtatgccgtcttctgcttg

SEQ ID NO: 421 - DNA, 65 bp, Artificial Sequence
  Synthetic Polynucleotide
tcaccgactgcccatagagaggactccagtcaccggtcaatatctcgtatgccgtcttctgcttg

SEQ ID NO: 422 - DNA, 65 bp, Artificial Sequence
  Synthetic Polynucleotide
tcaccgactgcccatagagaggactccagtcacgctgctacatctcgtatgccgtcttctgcttg

SEQ ID NO: 423 - DNA, 65 bp, Artificial Sequence
  Synthetic Polynucleotide
tcaccgactgcccatagagaggactccagtcacgcagtctaatctcgtatgccgtcttctgcttg

SEQ ID NO: 424 - DNA, 65 bp, Artificial Sequence
  Synthetic Polynucleotide
tcaccgactgcccatagagaggactccagtcactggaccacatctcgtatgccgtcttctgcttg

SEQ ID NO: 425 - DNA, 65 bp, Artificial Sequence
  Synthetic Polynucleotide
tcaccgactgcccatagagaggactccagtcacgtcacatcatctcgtatgccgtcttctgcttg

SEQ ID NO: 426 - DNA, 65 bp, Artificial Sequence
  Synthetic Polynucleotide
tcaccgactgcccatagagaggactccagtcacgttgctgaatctcgtatgccgtcttctgcttg

SEQ ID NO: 427 - DNA, 65 bp, Artificial Sequence
  Synthetic Polynucleotide
tcaccgactgcccatagagaggactccagtcacgagttagcatctcgtatgccgtcttctgcttg

SEQ ID NO: 428 - DNA, 65 bp, Artificial Sequence
  Synthetic Polynucleotide
tcaccgactgcccatagagaggactccagtcacacgatcatatctcgtatgccgtcttctgcttg

SEQ ID NO: 429 - DNA, 65 bp, Artificial Sequence
  Synthetic Polynucleotide
tcaccgactgcccatagagaggactccagtcaccgtcgtctatctcgtatgccgtcttctgcttg

SEQ ID NO: 430 - DNA, 65 bp, Artificial Sequence
  Synthetic Polynucleotide
tcaccgactgcccatagagaggactccagtcacgacatgcgatctcgtatgccgtcttctgcttg

SEQ ID NO: 431 - DNA, 65 bp, Artificial Sequence
  Synthetic Polynucleotide
tcaccgactgcccatagagaggactccagtcacgtgccataatctcgtatgccgtcttctgcttg

SEQ ID NO: 432 - DNA, 65 bp, Artificial Sequence
  Synthetic Polynucleotide
tcaccgactgcccatagagaggactccagtcaccgctaggaatctcgtatgccgtcttctgcttg

SEQ ID NO: 433 - DNA, 65 bp, Artificial Sequence
  Synthetic Polynucleotide
tcaccgactgcccatagagaggactccagtcaccgtaggtaatctcgtatgccgtcttctgcttg

SEQ ID NO: 434 - DNA, 65 bp, Artificial Sequence
  Synthetic Polynucleotide
tcaccgactgcccatagagaggactccagtcacagctagcgatctcgtatgccgtcttctgcttg

SEQ ID NO: 435 - DNA, 65 bp, Artificial Sequence
  Synthetic Polynucleotide
tcaccgactgcccatagagaggactccagtcactcctgtgcatctcgtatgccgtcttctgcttg

SEQ ID NO: 436 - DNA, 65 bp, Artificial Sequence
  Synthetic Polynucleotide
tcaccgactgcccatagagaggactccagtcacgtaatctgatctcgtatgccgtcttctgcttg

SEQ ID NO: 437 - DNA, 65 bp, Artificial Sequence
  Synthetic Polynucleotide
tcaccgactgcccatagagaggactccagtcacaacgtaggatctcgtatgccgtcttctgcttg

SEQ ID NO: 438 - DNA, 65 bp, Artificial Sequence
  Synthetic Polynucleotide
tcaccgactgcccatagagaggactccagtcacttcctgttatctcgtatgccgtcttctgcttg

SEQ ID NO: 439 - DNA, 65 bp, Artificial Sequence
  Synthetic Polynucleotide
tcaccgactgcccatagagaggactccagtcactgtccagtatctcgtatgccgtcttctgcttg

SEQ ID NO: 440 - DNA, 65 bp, Artificial Sequence
  Synthetic Polynucleotide
tcaccgactgcccatagagaggactccagtcacacaaggcaatctcgtatgccgtcttctgcttg

SEQ ID NO: 441 - DNA, 65 bp, Artificial Sequence
  Synthetic Polynucleotide
tcaccgactgcccatagagaggactccagtcacccttgaccatctcgtatgccgtcttctgcttg

SEQ ID NO: 442 - DNA, 65 bp, Artificial Sequence
  Synthetic Polynucleotide
tcaccgactgcccatagagaggactccagtcaccgcttgtgatctcgtatgccgtcttctgcttg

SEQ ID NO: 443 - DNA, 65 bp, Artificial Sequence
  Synthetic Polynucleotide
tcaccgactgcccatagagaggactccagtcactccaagcgatctcgtatgccgtcttctgcttg

SEQ ID NO: 444 - DNA, 65 bp, Artificial Sequence
  Synthetic Polynucleotide
tcaccgactgcccatagagaggactccagtcacctagtgacatctcgtatgccgtcttctgcttg

SEQ ID NO: 445 - DNA, 65 bp, Artificial Sequence
  Synthetic Polynucleotide
tcaccgactgcccatagagaggactccagtcacagaaccgtatctcgtatgccgtcttctgcttg

SEQ ID NO: 446 - DNA, 65 bp, Artificial Sequence
  Synthetic Polynucleotide
tcaccgactgcccatagagaggactccagtcactaattgcaatctcgtatgccgtcttctgcttg

SEQ ID NO: 447 - DNA, 65 bp, Artificial Sequence
  Synthetic Polynucleotide
tcaccgactgcccatagagaggactccagtcacctagtacaatctcgtatgccgtcttctgcttg

SEQ ID NO: 448 - DNA, 65 bp, Artificial Sequence
  Synthetic Polynucleotide
tcaccgactgcccatagagaggactccagtcacgctatatcatctcgtatgccgtcttctgcttg

SEQ ID NO: 449 - DNA, 65 bp, Artificial Sequence
  Synthetic Polynucleotide
tcaccgactgcccatagagaggactccagtcaccaatcggcatctcgtatgccgtcttctgcttg

SEQ ID NO: 450 - DNA, 65 bp, Artificial Sequence
  Synthetic Polynucleotide
tcaccgactgcccatagagaggactccagtcaccgatatcaatctcgtatgccgtcttctgcttg

SEQ ID NO: 451 - DNA, 65 bp, Artificial Sequence
  Synthetic Polynucleotide
tcaccgactgcccatagagaggactccagtcaccagtcaggatctcgtatgccgtcttctgcttg

SEQ ID NO: 452 - DNA, 65 bp, Artificial Sequence
  Synthetic Polynucleotide
tcaccgactgcccatagagaggactccagtcacgtaataatatctcgtatgccgtcttctgcttg

SEQ ID NO: 453 - DNA, 65 bp, Artificial Sequence
  Synthetic Polynucleotide
tcaccgactgcccatagagaggactccagtcacggagagatatctcgtatgccgtcttctgcttg

SEQ ID NO: 454 - DNA, 65 bp, Artificial Sequence
  Synthetic Polynucleotide
tcaccgactgcccatagagaggactccagtcacctctcataatctcgtatgccgtcttctgcttg

SEQ ID NO: 455 - DNA, 65 bp, Artificial Sequence
  Synthetic Polynucleotide
tcaccgactgcccatagagaggactccagtcaccagcgactatctcgtatgccgtcttctgcttg

SEQ ID NO: 456 - DNA, 65 bp, Artificial Sequence
  Synthetic Polynucleotide
tcaccgactgcccatagagaggactccagtcacggccaaggatctcgtatgccgtcttctgcttg

SEQ ID NO: 457 - DNA, 65 bp, Artificial Sequence
  Synthetic Polynucleotide
tcaccgactgcccatagagaggactccagtcacgcatatgcatctcgtatgccgtcttctgcttg

SEQ ID NO: 458 - DNA, 65 bp, Artificial Sequence
  Synthetic Polynucleotide
tcaccgactgcccatagagaggactccagtcacactaggatatctcgtatgccgtcttctgcttg

SEQ ID NO: 459 - DNA, 65 bp, Artificial Sequence
  Synthetic Polynucleotide
tcaccgactgcccatagagaggactccagtcacccttacctatctcgtatgccgtcttctgcttg

SEQ ID NO: 460 - DNA, 65 bp, Artificial Sequence
  Synthetic Polynucleotide
tcaccgactgcccatagagaggactccagtcactgttgacgatctcgtatgccgtcttctgcttg

SEQ ID NO: 461 - DNA, 65 bp, Artificial Sequence
  Synthetic Polynucleotide
tcaccgactgcccatagagaggactccagtcactacagttaatctcgtatgccgtcttctgcttg

SEQ ID NO: 462 - DNA, 65 bp, Artificial Sequence
  Synthetic Polynucleotide
tcaccgactgcccatagagaggactccagtcacttgttacgatctcgtatgccgtcttctgcttg

SEQ ID NO: 463 - DNA, 65 bp, Artificial Sequence
  Synthetic Polynucleotide
tcaccgactgcccatagagaggactccagtcactcgtgttgatctcgtatgccgtcttctgcttg

SEQ ID NO: 464 - DNA, 65 bp, Artificial Sequence
  Synthetic Polynucleotide
tcaccgactgcccatagagaggactccagtcacagtcaatgatctcgtatgccgtcttctgcttg

SEQ ID NO: 465 - DNA, 65 bp, Artificial Sequence
  Synthetic Polynucleotide
tcaccgactgcccatagagaggactccagtcactctgtagaatctcgtatgccgtcttctgcttg

SEQ ID NO: 466 - DNA, 65 bp, Artificial Sequence
  Synthetic Polynucleotide
tcaccgactgcccatagagaggactccagtcacgacaacgaatctcgtatgccgtcttctgcttg

SEQ ID NO: 467 - DNA, 65 bp, Artificial Sequence
  Synthetic Polynucleotide
tcaccgactgcccatagagaggactccagtcacccatggctatctcgtatgccgtcttctgcttg

SEQ ID NO: 468 - DNA, 65 bp, Artificial Sequence
  Synthetic Polynucleotide
tcaccgactgcccatagagaggactccagtcactgactctgatctcgtatgccgtcttctgcttg

SEQ ID NO: 469 - DNA, 65 bp, Artificial Sequence
  Synthetic Polynucleotide
tcaccgactgcccatagagaggactccagtcacaacgaggcatctcgtatgccgtcttctgcttg

SEQ ID NO: 470 - DNA, 65 bp, Artificial Sequence
  Synthetic Polynucleotide
tcaccgactgcccatagagaggactccagtcaccagaaggtatctcgtatgccgtcttctgcttg

SEQ ID NO: 471 - DNA, 65 bp, Artificial Sequence
  Synthetic Polynucleotide
tcaccgactgcccatagagaggactccagtcactgaagtcaatctcgtatgccgtcttctgcttg

SEQ ID NO: 472 - DNA, 65 bp, Artificial Sequence
  Synthetic Polynucleotide
tcaccgactgcccatagagaggactccagtcacatgttcctatctcgtatgccgtcttctgcttg

SEQ ID NO: 473 - DNA, 65 bp, Artificial Sequence
  Synthetic Polynucleotide
tcaccgactgcccatagagaggactccagtcacaagtggctatctcgtatgccgtcttctgcttg

SEQ ID NO: 474 - DNA, 65 bp, Artificial Sequence
  Synthetic Polynucleotide
tcaccgactgcccatagagaggactccagtcacggtacaatatctcgtatgccgtcttctgcttg

SEQ ID NO: 475 - DNA, 65 bp, Artificial Sequence
  Synthetic Polynucleotide
tcaccgactgcccatagagaggactccagtcacacaagtgcatctcgtatgccgtcttctgcttg

SEQ ID NO: 476 - DNA, 65 bp, Artificial Sequence
  Synthetic Polynucleotide
tcaccgactgcccatagagaggactccagtcactcacggtgatctcgtatgccgtcttctgcttg

SEQ ID NO: 477 - DNA, 65 bp, Artificial Sequence
  Synthetic Polynucleotide
tcaccgactgcccatagagaggactccagtcacttgcgttaatctcgtatgccgtcttctgcttg

SEQ ID NO: 478 - DNA, 65 bp, Artificial Sequence
  Synthetic Polynucleotide
tcaccgactgcccatagagaggactccagtcacttgtagccatctcgtatgccgtcttctgcttg

SEQ ID NO: 479 - DNA, 65 bp, Artificial Sequence
  Synthetic Polynucleotide
tcaccgactgcccatagagaggactccagtcactcaccggaatctcgtatgccgtcttctgcttg

SEQ ID NO: 480 - DNA, 65 bp, Artificial Sequence
  Synthetic Polynucleotide
tcaccgactgcccatagagaggactccagtcaccgcgcaagatctcgtatgccgtcttctgcttg

(19) United States
(12) Patent Application Publication
(10) Pub. No.: US 2017/0029813 A1
(43) Pub. Date: Feb. 2, 2017

(54) METHODS AND SYSTEMS FOR DROPLET TAGGING AND AMPLIFICATION

(71) Applicants: President and Fellows of Harvard College, Cambridge, MA (US); The General Hospital Corporation d/b/a Massachusets General Hospital, Boston, MA (US)

(72) Inventors: David A. Weitz, Bolton, MA (US); Anthony John Iafrate, Newton, MA (US); Zongli Zheng, Boston, MA (US); Huidan Zhang, Cambridge, MA (US)

(21) Appl. No.: 15/303,893

(22) PCT Filed: Apr. 17, 2015
(86) PCT No.: PCT/US15/26422

§ 371 Date: Oct. 13, 2016

Related U.S. Application Data
(60) Provisional application No. 61/981,108, filed on Apr. 17, 2014.

(51) Int. Cl. C12N 15/10

(52) U.S. Cl. CPC C12N 15/1065

(57) ABSTRACT

The present invention generally relates to microfluidic devices, including methods and systems for tagging droplets within such devices. In some aspects, microfiuidic droplets are manipulated by exposing the droplets (or other discrete entities) to a variety of different conditions. By incorporating into the droplets a plurality of nucleic acid “tags,” and optionally amplifying the nucleic acids, e.g., within the droplets, the conditions that a droplet was exposed to may be encoded by the nucleic acids. Thus, even if droplets exposed to different conditions are mixed together, the conditions that each droplet encountered may still be determined, for example, by sequencing the nucleic acids.

### RELATED APPLICATIONS

This application claims the benefit of U.S. Provisional Patent Application Ser. No. 61/981,108, filed Apr. 17, 2014, entitled “Methods and Systems for Droplet Tagging and Amplification,” by Weitz, et al., incorporated herein by reference in its entirety.

### FIELD

The present invention generally relates to microfluidic devices, including methods and systems for tagging droplets within such devices.

### BACKGROUND

A variety of techniques exist for producing fluidic droplets within a microfluidic system, such as those disclosed in Int. Pat. Pub. Nos. WO 2004/091763, WO 2004/002627, WO 2006/096571, WO 2005/021151, WO 2010/033200, and WO 2011/056546, each incorporated herein by reference in its entirety. In some cases, relatively large numbers of droplets may be produced, and often at relatively high speeds, e.g., the droplets may be produced at rates of least about 10 droplets per second. The droplets may also contain a variety of species therein. However, it can be difficult to accurately track such droplets, especially when large numbers of droplets are produced and/or the droplets are produced at very high rates. In addition, such tracking may be complicated if the droplets are exposed to a variety of different conditions, contain different species, etc., such that the droplets are not all identical.

### SUMMARY

The present invention generally relates to microfluidic devices, including methods and systems for tagging droplets within such devices. The subject matter of the present invention involves, in some cases, interrelated products, alternative solutions to a particular problem, and/or a plurality of different uses of one or more systems and/or articles.

In one aspect, the present invention is generally directed to a method. According to one set of embodiments, the method comprises acts of exposing a microfluidic droplet to a first condition and adding a first nucleic acid to the droplet, where the first nucleic acid encodes the first condition, exposing the microfluidic droplet to a second condition and adding a second nucleic acid to the droplet, where the second nucleic acid encodes the second condition, and amplifying the first nucleic acid and the second nucleic acid within the microfluidic droplet.

The method, in another set of embodiments, is generally directed to acts of exposing a plurality of microfluidic droplets to a plurality of conditions such that substantially each microfluidic droplet is sequentially exposed to at least two different conditions, where when a microfluidic droplet is exposed to a condition, a nucleic acid encoding the condition is added to the microfluidic droplet, and amplifying the nucleic acids within at least some of the microfluidic droplets.

In another aspect, the present invention is generally directed to an article. In accordance with one set of embodiments, the article comprises a plurality of microfluidic droplets, at least some of the droplets containing one or more primers and one or more nucleic acids encoding a plurality of conditions that the at least some droplets were exposed to.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C schematically illustrate various methods for tagging droplets, in accordance with various embodiments of the invention;

FIGS. 2A-2B schematically illustrates joining together various nucleic acid tags, in certain embodiments of the invention;

FIG. 3 illustrates a schematic for PCR amplification within droplets, according to some embodiments of the invention;

FIG. 4 illustrates evenly amplified nucleic acids, in another embodiment of the invention;

FIG. 5 illustrates a schematic for PCR amplification within droplets, according to some embodiments of the invention;

FIGS. 6A-6B illustrate amplified nucleic acids, in yet another embodiment of the invention;

FIGS. 7A-7F illustrate apoptosis detection of cells contained within droplets, in accordance with one set of embodiments of the invention;

FIG. 8 illustrates amplification of nucleic acids in the presence of an apoptosis assay reagent, in another set of embodiments of the invention;

FIG. 9 illustrates a schematic for PCR amplification within droplets, according to another embodiment of the invention;

FIGS. 10A-10D illustrates the design of a microfluidic device used in one embodiment of the invention;

FIG. 11 illustrates the sequences used in one embodiment of the invention;

FIGS. 12A-12D illustrates cells in agarose particles, in accordance with one embodiment of the invention;

FIGS. 13A-13B illustrate cell detection using flow cytometry, in another embodiment of the invention;

FIGS. 14A-14D illustrate fluorescence microscope images of cells, in yet another embodiment of the invention;

FIGS. 15A-15D illustrate fluorescence microscope images of cells, in still another embodiment of the invention;

FIGS. 16A-16B illustrate streptavidin magnetic beads in agarose gel particles, in accordance with yet another embodiment of the invention; and

FIG. 17 illustrates barcoded beads, in another embodiment of the invention.

### DETAILED DESCRIPTION

The present invention generally relates to microfluidic devices, including methods and systems for tagging droplets within such devices. In some aspects, microfluidic droplets are manipulated by exposing the droplets (or other discrete entities) to a variety of different conditions. By incorporating into the droplets a plurality of nucleic acid “tags,” and optionally amplifying the nucleic acids, e.g., within the droplets, the conditions that a droplet was exposed to may be encoded by the nucleic acids. Thus, even if droplets exposed to different conditions are mixed together, the conditions that each droplet encountered may still be determined, for example, by sequencing the nucleic acids.

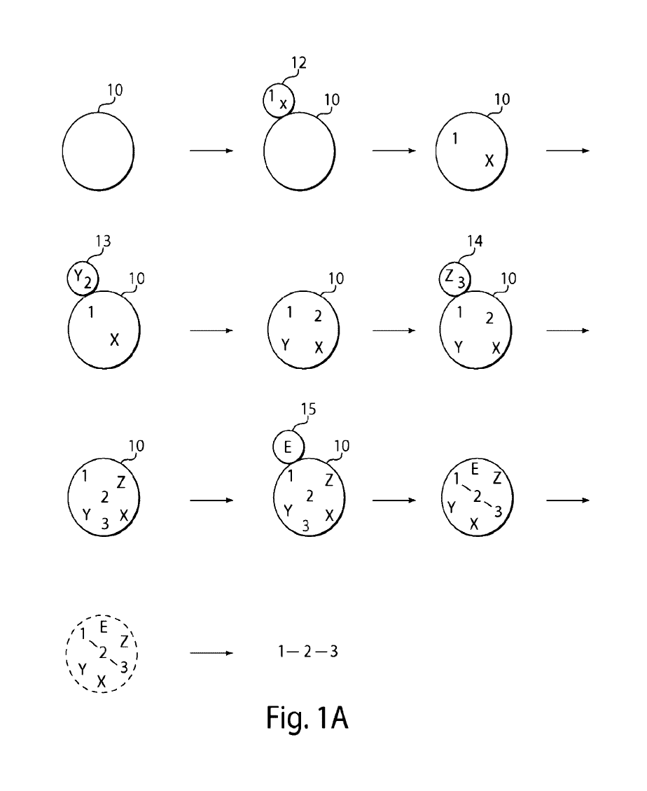

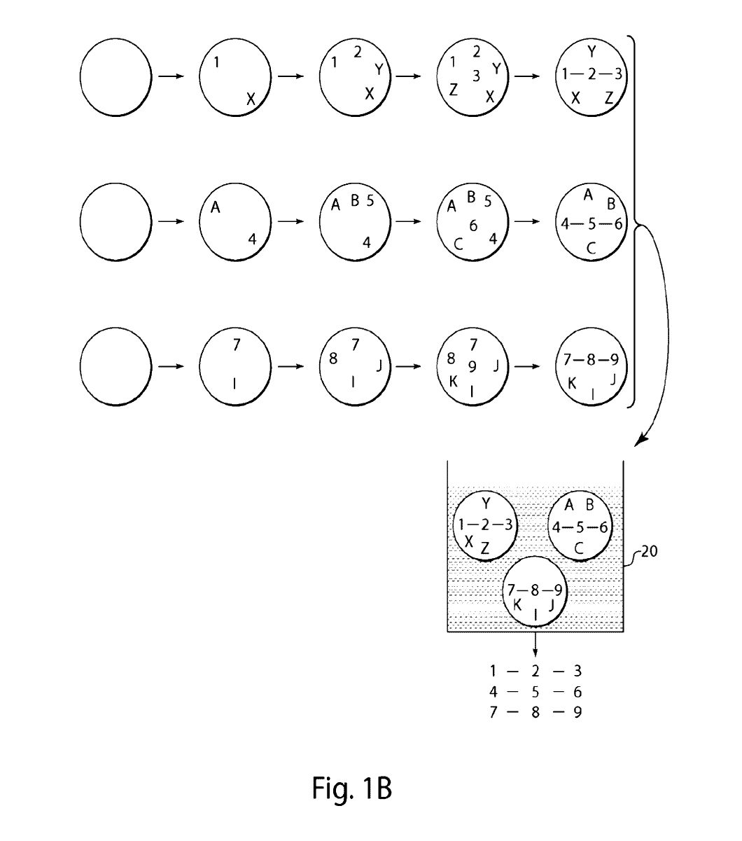

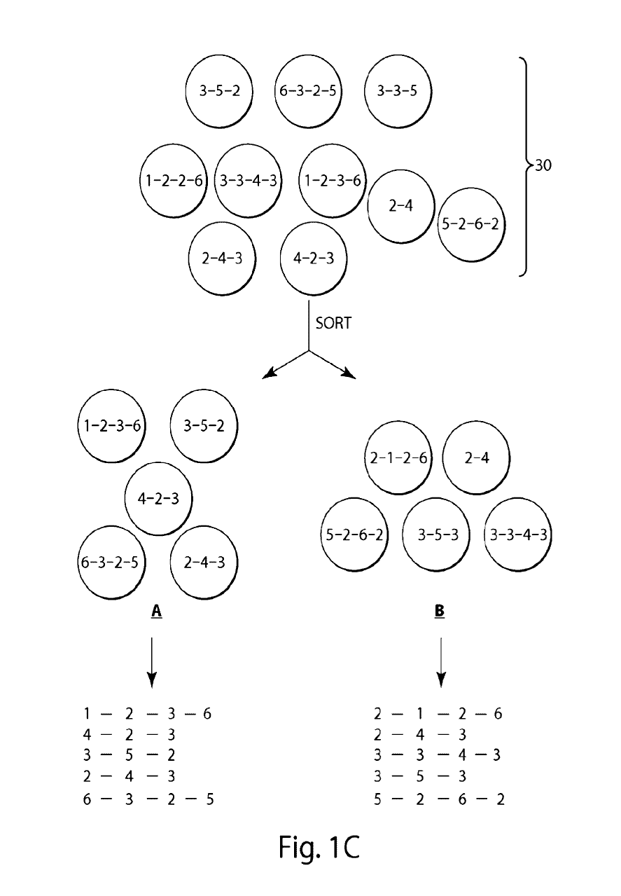

Initially, certain non-limiting examples are discussed with reference to FIG. 1. However, in other embodiments, other configurations may be used as well. Turning first to FIG. 1A, a droplet is shown that is exposed to a variety of conditions (for example, a species such as a drug), and each time the droplet is exposed to a condition, a nucleic acid encoding the condition is added to the droplet. It should be noted that the condition need not be the addition of a chemical species, but could also be a physical condition, such as exposure to a particular temperature. It should further be noted that although droplets are discussed with reference to FIG. 1, this is for ease of presentation only; in other embodiments, other discrete entities may be used instead of these droplets, for example, microwells in a microwell plate.

As is shown in this example, droplet 10 first encounters, and is fused with, droplet 12 containing species X and nucleic acid 1. Subsequently, droplet 10 encounters and is fused with droplet 13 (containing species Y and nucleic acid 2) and droplet 14 (containing species Z and nucleic acid 3). An enzyme E may then be introduced in some fashion into droplet 10, for example, as part of another droplet fusion event (as is shown here with droplet 15), or the enzyme may initially be found in any one of droplets 10, 12, 13, or 14. The enzyme may be used to join nucleic acids 1, 2, and 3 together, as is shown with nucleic acids 1-2-3. For instance, in one set of embodiments, the enzyme may be a polymerase (such as Taq polymerase), and a PCR reaction used to join nucleic acids 1, 2, and 3 together to from a DNA “barcode.” The droplet can then be burst to access the joined nucleic acid, and the nucleic acid can be sequenced or otherwise determined to determine the history of droplet 10.

In some embodiments, two or more droplets may be treated in such a fashion, as is shown in FIG. 1B. For instance, two or more droplets may be exposed to a variety of different conditions, where each time a droplet is exposed to a condition, a nucleic acid encoding the condition is added to the droplet. However, even if such droplets with different histories are later combined (e.g., in a mixture), as is shown in container 20, the conditions each of the droplets was exposed to is still determinable through the different nucleic acids contained in each droplet. This is true even if the droplets themselves are burst and/or the contents are mixed together. For example, as is shown in FIG. 1B, by determining nucleic acids 1-2-3, 4-5-6, and 7-8-9, one knows that at least one droplet in container 20 was exposed to conditions A, B, and C, at least one droplet was exposed to conditions I, J, and K, and at least one droplet was exposed to conditions X, Y, and Z. In addition, one may also be able to determine that no droplets in container 20 were exposed to other sets of conditions. For example, one may be able to determine that no droplet was exposed to conditions X, Y, and K, as there is no sequence 1-2-9 present in container 20.

As is shown in FIG. 1C, in certain embodiments of the invention, testing and/or sorting of the droplets may also occur. As shown in this figure, a plurality of droplets exposed to different conditions (as encoded by the nucleic acids contained therein, represented by various numbers) may be sorted based on some criteria. For example, if the droplet further contain cells, then a property of the cell (e.g., whether the cell is alive or dead) may be used to sort the droplets into 2 (or more) populations. Each population can then be separately analyzed, e.g., by determining the nucleic acids contained therein, to determine which conditions may have led to the different populations. It should be noted that although cells are used here, this is by way of example only, and that other species may be determined, instead of or in addition to cells. In the example of FIG. 1C, a population of droplets 30 was sorted into a first group (A) and a second group (B), whether using cells or other suitable species. The nucleic acids in each group can then be sequenced. Thus, for example, it may be determined that the members of Group A each have conditions 2 and 3 in common, while the members of Group B do not have conditions 2 and 3 in common (although conditions 2 or 3 may be separately present in some of the Group B droplets). Thus, these sorting experiments would demonstrate that the combination of conditions 2 and 3 is necessary for some effect to occur (Group A), and if both are not present, the effect does not occur (Group B).

Also noteworthy is that in FIG. 1C, the number of conditions each droplet is exposed to is not necessarily the same; instead, the conditions may vary, i.e., intentionally, or due to experimental errors or uncertainty. For example, droplet 2-4 was exposed to conditions 2 and 4, while droplet 1-2-3-6 was exposed to conditions 1, 2, 3, and 6. Furthermore, it should also be noted that, due to the joining of the nucleic acids prior to removing the nucleic acids from the droplets, the conditions each droplet was exposed to may be separately maintained and analyzed, even if the droplets and/or the nucleic acids are subsequently mixed together. Thus, with reference to FIG. 1C, if the nucleic acids were not joined together, then each of Groups A and B would appear to be identical, since each group contains the same numbers (representing conditions) in exactly the same proportions in this illustrative example.

Thus, one surprising feature of certain embodiments of the invention is that the joined nucleic acids allow more complex conditions to be readily determined, for example a single condition or multiple conditions. This can be accomplished without loss of information even if the droplets are burst or otherwise combined together, e.g., for ease of processing or analysis. In contrast, in techniques in which various tags are separately introduced into droplets, without combining the tags together, the conditions cannot be so readily analyzed; instead, care must be taken in keeping each of the droplets separate, as any tags that are accidently combined (for example by bursting or fusing different droplets together) would result in loss of information.

The above discussion is not intended to be limiting; other embodiments of the invention are also possible for tagging droplets, as will now be discussed. For instance, various aspects of the present invention are generally directed to systems and methods for tagging or identifying droplets within microfluidic devices, e.g., using nucleic acids and other “tags,” that can be bound together. By binding the tags together, information about the droplet containing the tag may be retained, e.g., even after the tag is separated from the droplet and/or combined with other, different tags. Thus, for example, a plurality of tags from different droplets (e.g., exposed to different conditions) may be combined and analyzed together.

Certain aspects of the present invention involve the use of a plurality of droplets or other discrete entities or compartments, e.g., where the contents of the entities are not readily mixed with the contents of other entities. For example, the discrete entities may be droplets contained within a carrying fluid, microwells of a microwell plate, individual spots on a slide or other surface, or the like. As discussed herein, each of the entities may a specific location that can contain one or more tags or other species, without accidental mixing with other entities. The entities may be relatively small in some cases, for example, each entity may have a volume of less than about 1 ml, less than about 300 microliters, less than about 100 microliters, less than about 30 microliters, less than about 10 microliters, less than about 3 microliters, less than about 1 microliter, less than about 500 nl, less than about 300 nl, less than about 100 nl, less than about 50 nl, less than about 30 nl, or less than about 10 nl.

In some embodiments, the droplet or other entity may contain various species, e.g., cells, chemicals, or the like. Other examples of species are discussed herein. For example, if the droplet or other entity contains one or more cells, the cells may be substantially identical or different. For example, a droplet or other entity may contain more than one cell or other species), where the cells (or other species) are the same or different; the cells (or other species) in different droplets or entities may also be the same or different. If cells are used, the cells may also be, in some embodiments, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like.

Thus, as non-limiting examples, the effects of one or more conditions on a specific type of cell (or specific types of cells) may be studied. As another example, the effects of a certain specific condition (or conditions) on a suitable population of cells may be studied. It addition, it should be noted that the present invention is not limited to only study of cells. In other embodiments, for example, the droplets or other entities may contain species in which a variety of conditions is to be applied. For example, the species may be a chemical reagent, e.g., one that is biological or nonbiological, organic or inorganic, etc. For instance, the species may be a polymer, a nucleic acid, a protein, a drug, a small molecular compound (e.g., having a molecular weight of less than about 1000 Da or less than about 2000 Da), an antibody, an enzyme, a peptide, or the like. Other examples of species are discussed in more detail below.

One or more tags may be present within a droplet (or other entity), which can be analyzed to determine the identity and/or history of the droplet. In some cases, the tags may be chosen to be relatively inert relative to other components of the droplet or other entity. The tags may be present initially in the droplet or other entity, and/or subsequently added, e.g., using processes such as those described below. For instance, tags may be added when the droplet or other entity is exposed to one or more conditions (or proximate in time to such exposure). In some cases, more than one tag may be present in a droplet or other entity.

In certain embodiments of the invention, the tags within a droplet or other entity can be joined together, for example, chemically, to produce a joined tag. Any suitable technique may be used to join tags together, e.g., prior to removal from the droplet or entity. The tags may be joined using any suitable technique. For example, the tags may be joined together using an enzyme, a catalyst, or a reactant, which may be added to the droplet or other entity using any suitable technique. For instance, a droplet containing the tags may be fused to another droplet containing the chemical agent, or a chemical reactant may be added or inserted into a droplet or other entity, for example, using pipetting or other techniques, and in some cases, using automated techniques.

By joining the tags in a droplet (or other entity) together to produce a joined tag, the identity and/or history of the droplet may be maintained by maintaining the joined tags, even if the tags are separated from the droplet or tags from different droplets are mixed together. For example, joined tags from a variety of droplets or entities can be collected together and analyzed. In some embodiments, a series of droplets or other entities may be separated into various groups depending on various properties, and the tags within each group may be manipulated together and/or used to identify such droplets or entities having such properties.

The tags may include, for example, nucleic acids, which may be joined together. In one set of embodiments, the nucleic acids may be joined together using enzymes. For instance, in certain embodiments, the nucleic acids together are joined together using ligases. Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, Taq DNA Ligase, or the like. Many such ligases may be purchased commercially. As additional examples, in some embodiments, two or more nucleic acids may be ligated together using annealing or a primer extension method.

In yet another set of embodiments, the nucleic acids may be joined together and or amplified using PCR (polymerase chain reaction) or other amplification techniques. Typically, in PCR reactions, the nucleic acids are heated to cause dissociation of the nucleic acids into single strands, and a heat-stable DNA polymerase (such as Taq polymerase) is used to amplify the nucleic acid. This process is often repeated multiple times to amplify the nucleic acids.

In one set of embodiments, the PCR may be performed within the droplets. For example, the droplets may contain a polymerase (such as Taq polymerase), and DNA nucleotides, and the droplets may be processed (e.g., via repeated heated and cooling) to amplify the nucleic acid within the droplets. The polymerase and nucleotides may be added at any suitable point, e.g., before, during, or after various nucleic acids encoding various conditions are added to the droplets. For instance, as a non-limiting example, droplet 15 in FIG. 1A may contain polymerase and DNA nucleotides, which is fused to droplet 10 to allow amplification to occur. Those of ordinary skill in the art will be aware of suitable PCR techniques and variations, such as assembly PCR or polymerase cycling assembly, which may be used in some embodiments to produce an amplified nucleic acid. Non-limiting examples of such procedures are also discussed below. In addition, in some cases, suitable primers may be used to initiate polymerization, e.g., P5 primer, P7 primer, PE1 primer, PE2 primer, A19 primer, or other primers known to those of ordinary skill in the art. In some embodiments, primers may be added to the droplets, or the primers may be present on one or more of the nucleic acids within the droplets. Those of ordinary skill in the art will be aware of suitable primers, many of which can be readily obtained commercially.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer may be complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

Other non-limiting examples of amplification methods known to those of ordinary skill in the art that may be used include, but are not limited to, reverse transcriptase (RT) PCR amplification, in vitro transcription amplification (IVT), multiple displacement amplification (MDA), or quantitative real-time PCR (qPCR).

By amplifying the nucleic acids within the droplets, e.g., prior to releasing the nucleic acids from the droplets, “even” amplification of the various nucleic acids may be achieved in some embodiments of the invention. Generally, in “even” amplification, approximately the same amount of nucleic acids may be produced within each droplet. In contrast, if a variety of nucleic acids are mixed together then amplified, differences in reaction rate between the various nucleic acids during PCR may result in some nucleic acids being amplified over other nucleic acids. However, according to certain embodiments, the nucleic acids within a plurality of droplets may be amplified such that the number of nucleic acid molecules for each type of nucleic acid may have a distribution such that no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of nucleic acid molecules per droplet.

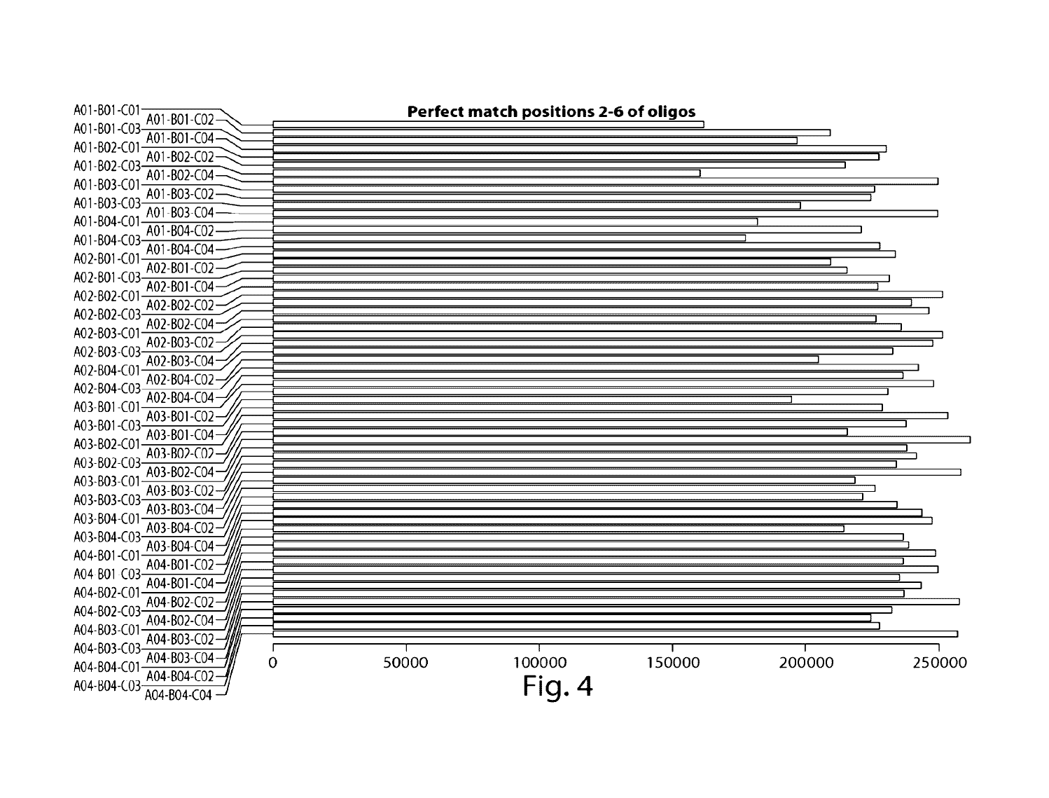

In some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet. See, e.g., FIG. 4 for an example of a population of nucleic acid molecules that have been evenly amplified within droplets.

As discussed herein, various sequences of nucleic acids can be used to encode specific conditions that a droplet or other entity may be exposed to, and such nucleic acids can be added thereto to indicate such exposure to a condition, in accordance with certain embodiments. In some cases, the nucleic acids within a droplet or other entity may be joined together prior to removal (for example, upon bursting of a droplet, washing of a slide, etc.). Different nucleic acids from different droplets or entities may be mixed together; however, even after such mixing, each nucleic acid can be individually sequenced to determine the specific conditions that the corresponding droplet or entity had been exposed to.

Any suitable system may be used for encoding. For example, in one set of embodiments, a nucleic acid tag may include an encoding region of nucleotides, and optionally a connecting region. The nucleotides in the encoding region may correspond to a specific condition (or set of conditions). Any suitable number of conditions may be arbitrarily encoded in such a fashion, where the number of conditions that are encodable by such an encoding region may be determined by the number of nucleotides in the encoding region. Thus, for instance, an encoding region having length n can encode up to 4ⁿ regions (based on the four types of nucleotides). For example, a first condition may be encoded with A, a second condition may be encoded with T (or U if the nucleic acid is an RNA), a third condition may be encoded with G, a fourth condition may be encoded with C, etc. As a more complex example, an encoding region containing 3 nucleotides is sufficient to encode over 50 different conditions (since 4³=64). One or more than one encoding region may be used. In addition, the encoding region may also include other nucleotides used for error detection and/or correction, redundancy, or the like, in certain embodiments.

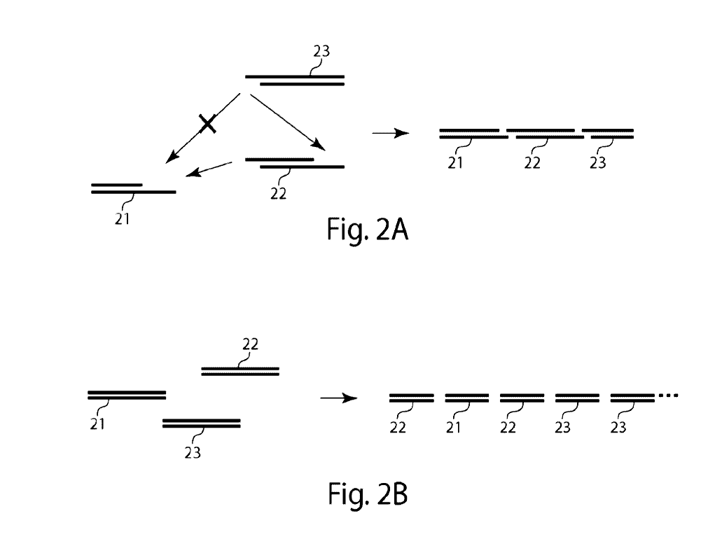

A nucleic acid tag may also include, in some cases, one or more connecting regions, which are joined together. For example, the connecting regions may include “sticky ends,” or overhangs of nucleic acids, such that only specific nucleic acids can be properly joined together. For instance, as is shown in FIG. 2A, a first nucleic acid tag 21 (encoding a first condition) may include a first sticky end that is substantially complementary to a sticky end on second nucleic acid tag 22 but not to a sticky end on third nucleic acid tag 23; similarly, second nucleic acid 22 (encoding a second condition) may include a sticky end that is substantially complementary to a sticky end on third nucleic acid tag 23 (encoding a third condition) but not to the sticky end on first nucleic acid 21. Thus, upon exposure to suitable ligases, the first, second, and third nucleic acids may be joined together in an order suitable for subsequent study, without the nucleic acids being incorrectly joined together in an incorrect order (e.g., a first nucleic acid being joined to another first nucleic acid). Accordingly, by sequencing the final joined nucleic acid, it can be determined that this nucleic acid was in a droplet or other entity exposed to the first, second, and third conditions.

However, it should be understood that in other embodiments, there may be no need to ensure that the nucleic acid tags are joined together in a certain configuration or order. For example, as is shown in FIG. 2B, nucleic acids 21, 22, and 23 may be joined together in any suitable order; and the resulting nucleic acid may be analyzed to determine that that the nucleic acid encoded the conditions encoded by nucleic acids 21, 22, and 23.

The nucleic acid tag may also have any suitable length or number of nucleotides, depending on the application. For example, a nucleic acid tag may have a length shorter or longer than 10 nt, 30 nt, 50 nt, 100 nt, 300 nt, 500 nt, 1000 nt, 3000 nt, 5000 nt, or 10,000 nt, etc. In some cases, other portions of the nucleic acid tag may also be used for other purposes, e.g., in addition to encoding conditions. For example, portions of the nucleic acid tag may be used to increase the bulk of the nucleic acid tag (e.g., using specific sequences or nonsense sequences), to facilitate handling (for example, a tag may include a poly-A tail), to increase selectivity of binding (e.g., as discussed below), to facilitate recognition by an enzyme (e.g., a suitable ligase), to facilitate identification, or the like. The nucleic acid tag may comprise DNA, RNA, and/or other nucleic acids such as PNA, and/or combinations of these and/or other nucleic acids. In some cases, the nucleic acid tag is single stranded, although it may be double stranded in other cases.

In some cases, the nucleic acid tag may have a length of at least about 10 nt, at least about 30 nt, at least about 50 nt, at least about 100 nt, at least about 300 nt, at least about 500 nt, at least about 1000 nt, at least about 3000 nt, at least about 5000 nt, at least about 10,000 nt, etc. In some cases, the nucleic acid tag may have a length of no more than about 10,000 nt, no more than about 5000 nt, no more than about 3000 nt, no more than about 1000 nt, no more than about 500 nt, no more than about 300 nt, no more than about 100 nt, no more than about 50 nt, etc. Combinations of any of these are also possible, e.g., the nucleic acid tag may be between about 10 nt and about 100 nt. The length of the nucleic acid tag is not critical, and a variety of lengths may be used in various embodiments.

As mentioned, in certain aspects of the invention, one or more conditions may be encoded using such tags, e.g., added to droplets or other entities exposed to such conditions. Thus, for example, a droplet may be exposed to a first condition and a first tag added to the droplet, then the droplet may be exposed to a second condition and a second tag added to the droplet, then the droplet may be exposed to a third condition and a third tag added to the droplet, then the droplet may be exposed to a fourth condition and a fourth tag added to the droplet, etc. Accordingly, any number of conditions may be present, e.g., the droplet or other entity may be exposed to 2 3, 4, 5, 6, 7, 8, 9, 10, or more conditions. The tags may also be combined together (e.g., joined together) to produce a joined tag that encodes the history and/or identity of the droplet or entity, as previously discussed.

Any suitable conditions may be encoded by the tags. For example, in one set of embodiments, the identity of the droplet or other entity may be encoded using one or more tags. For example, each droplet or entity may be assigned a unique tag, or a unique combination of tags. As another example, the condition may be a species that a droplet or other entity is exposed to, internally and/or externally. Any of a wide variety of species may be encoded by a suitable tag. For example, the species may be a drug (or a suspected drug), a cell, a polymer, a peptide, a protein, an enzyme, a hormone, an antibiotic, a vitamin, a carbohydrate, a sugar, an antibody, a reagent, a gas, a dye, an ion, a virus, a bacterium, pH level (e.g., acidic or basic conditions), or the like. Thus, for example, a plurality of cells, or a plurality of cell types may be encoded using a unique tag, or a unique combination of tags. Any number of tags may be used to encode such conditions, depending on the application. For example, there may be at least 10, at least 30, at least 50, at least 100, at least 300, at least 500, at least 1,000, at least 3,000, at least 5,000, at least 10,000, at least 30,000, at least 50,000, at least 100,000, or more unique tags, e.g., substantially encoding a like number of suitable conditions such as any of those described herein. For instance, as a first condition, a library of droplets, containing a plurality of cells or cell types, may be encoded such that substantially each droplet contains one cell or one cell type, and an associated tag, such as a nucleic acid.

In addition, the condition may also be an external physical condition in some instances. For example, the droplet or other entity may be exposed to an external physical condition such as a certain or predetermined temperature, pressure, electrical condition (e.g., current, voltage, etc.), etc. and a suitable tag may be introduced into the droplet or entity before, during, or after exposure to the external physical condition. As yet another example, the condition may be a processing condition, for example, filtration, sedimentation, centrifugation, etc.

In one set of embodiments, the condition may be a condition used to lyse cells that may be contained within the droplets. For example, the condition could be exposure to a lysing chemical (e.g., pure water, a surfactant such as Triton-X or SDS, an enzyme such as lysozyme, lysostaphin, zymolase, cellulase, mutanolysin, glycanases, proteases, mannase, proteinase K, etc.), or a physical condition (e.g., ultrasound, ultraviolet light, mechanical agitation, etc.). In some cases, lysing a cell will cause the cell to release its contents, e.g., cellular nucleic acids, proteins, enzymes, sugars, etc. In some embodiments some of the cellular nucleic acids may also be joined to one or more nucleic acids contained within the droplet. For example, in one set of embodiments, RNA transcripts typically produced within the cells may be released and then joined to the nucleic acid tags.

Combinations of any of these and/or other conditions are also contemplated. For example, as noted above, a droplet may be exposed to a first condition encoded by a first tag, a second condition encoded by a second tag, a third condition encoded by a third tag, etc. The conditions may be the same or different. In addition, a droplet or other entity may be exposed to any number of conditions (e.g. 1, 2, 3, 4, 5, 6, etc.), and different droplets or entities need not be exposed to the same conditions, or the same number of conditions. In addition, in some embodiments, a plurality of droplets (or other entities) may be randomly exposed to a plurality of conditions, e.g., such that not all droplets are exposed to all conditions. As noted above, when a droplet or other entity is exposed to a condition, a nucleic acid encoding the condition may be added to the droplet or entity, such that the specific history of the droplet or entity need not be predetermined, and can be randomly determined. For instance, a plurality of initial, substantially identical droplets (or other entities) may be exposed to a plurality of second droplets containing different species (and/or one or more species at different concentrations), such that each initial droplet is randomly fused to one or more second droplets.

In addition, in some embodiments, droplets or other entities may be separated or sorted into various groups depending on various properties, and the tags within each group may be manipulated together and/or used to identify such droplet or entities having such properties. Thus, as non-limiting examples, a droplet (or other entity) may be sorted into a first group or a second group depending on whether a reaction occurred within the droplet or not, the droplet may be sorted based on a property of a cell or other species contained within the droplet, etc. The sorting may also occur into two or more groups.

In some cases, some or all of the groups of droplets (or other entities) may be analyzed, e.g., using the tags contained therein, to determine characteristics of the droplets or entities within those groups, and/or to determine characteristics of the droplets or entities within a group that separates that group from other groups. For instance, members of a group may have, in common, one or more tags, and/or one or more specific combinations of tags, that separates those group members from other group members, e.g., as was explained above with respect to FIG. 1C. Such tags may be used, for example, to identify or distinguish one or more conditions that result in a particular outcome from conditions that do not result in that outcome.

Thus, in one set of embodiments, a property of a droplet or other entity is determined, and the droplet or other entity is sorted based on that property. The property may also be a property of a species contained within the droplet or entity, such as a cell. The property may be any physical or chemical property that can be determined. For instance, properties such as fluorescence, transparency, density, size, volume, etc., of a droplet or other entity (or species contained therein) may be determined, and used for sorting purposes. In some cases, one or more reagents may also be added to the droplets or other entities, e.g., to start or facilitate a reaction that can be determined, e.g., for sorting purposes.

In some cases, the droplets or other entities may be burst or disrupted in order to access the tags. This may occur at any suitable time, e.g., before or after joining of the tags together. For example, droplets contained in a carrying fluid may be disrupted using techniques such as mechanical disruption or ultrasound. Similarly, entities on a surface or droplets may be disrupted using exposure to chemical agents or surfactants (for example, 1H,1H,2H,2H-perfluorooctanol), or washed or rinsed to collect the tags.

The tags may then be determined to determine the identity and/or history of the droplet (or other entity), e.g., to determine conditions that the droplet or other entity was exposed to. Any suitable method can be used to determine the tags, depending on the type of tags used. For example, fluorescent particles may be determined using fluorescence measurements, or nucleic acids may be sequenced using a variety of techniques and instruments, many of which are readily available commercially. Examples of such techniques include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing, nanopore sequencing, microfluidic Sanger sequencing, digital RNA sequencing (“digital RNA-seq”), etc.

In addition, in some cases, one or more other species may be associated with the tags, e.g., covalently bound or otherwise joined thereto. For example, as previously discussed, in some cases, nucleic acids released by a lysed cell may be joined to one or more tags. These may include, for example, chromosomal DNA, RNA transcripts, tRNA, mRNA, mitochondrial DNA, or the like. Such nucleic acids may be sequenced, in addition to sequencing the tags themselves, which may yield information about the nucleic acid profile of the cells, which can be associated with the tags, or the conditions that the corresponding droplet or cell was exposed to. Thus, as a non-limiting example, RNA transcripts from the cell may be joined to one or more tags, which may be sequenced and correlated with conditions that the corresponding droplet or cell to determine information such as apoptosis gene signatures, growth arrest signatures, immune signatures, metabolic gene sets, or expression of genes that confer susceptibility or resistance to other known agents, etc.

As another example, in some cases, the tags may be bound to particles, e.g., to facilitate identification, collection, or the like. For example, the tags may include a sequence or a moiety that can be used to bind to a particle. For instance, the tag may contain an acrylic phosphoramidite at its 5′ end can be incorporated into a polyacrylamide mesh of certain particles during the polymerization process. As another example, acrydite-modified oligonucleotide tags can react covalently with thiol groups and thus, particles having thiol groups would bind acrydite-modified oligonucleotides. In another example, oligonucleotide tags having amino groups can be covalently bound to the carboxy group of certain particles. In yet another example, oligonucleotides with tag a biotin group can be attached to streptavidin-coated tags. In yet another example, the particle may include antibodies or antibody fragments able to recognize certain oligonucleotide sequences present on the tags. Therefore, different types of incorporation of nucleic acids into/onto the particles are possible.

The particle may be, in one set of embodiments, a hydrogel particle or a colloidal particle (polystyrene, magnetic or polymer particle, etc.). See, e.g., Int. Pat. Apl. Pub. No. WO 2008/109176, entitled “Assay and other reactions involving droplets” (incorporated herein by reference) for examples of hydrogel particles, including hydrogel particles containing DNA. The microspheres may be porous in some embodiments. Examples of hydrogels include, but are not limited to agarose or acrylamide-based gels, such as polyacrylamide, poly-N-isopropylacrylamide, or poly N-isopropylpolyacrylamide. For example, an aqueous solution of a monomer may be dispersed in a droplet, and then polymerized, e.g., to form a gel. Another example is a hydrogel, such as alginic acid that can be gelled by the addition of calcium ions. In some cases, gelation initiators (ammonium persulfate and TEMED for acrylamide, or Ca²⁺ for alginate) can be added to a droplet, for example, by co-flow with the aqueous phase, by co-flow through the oil phase, or by coalescence of two different drops, e.g., as discussed in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007; or in U.S. patent application Ser. No. 11/698,298, filed Jan. 24, 2007, entitled “Fluidic Droplet Coalescence,” by Ahn, et al.; each incorporated herein by reference in their entireties.

In another set of embodiments, the particles may comprise one or more polymers. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. In addition, in some cases, the particles may be magnetic, which could allow for the magnetic manipulation of the particles. For example, the particles may comprise iron or other magnetic materials. The particles could also be functionalized so that they could have other molecules attached, such as proteins, nucleic acids or small molecules. Thus, some embodiments of the present invention are directed to a set of particles defining a library of, for example, nucleic acids, proteins, small molecules, or other species such as those described herein. In some embodiments, the particle may be fluorescent.

The particle is a microparticle in certain aspects of the invention. The particle may be of any of a wide variety of types; as discussed, the particle may be used to bind a particular oligonucleotide tag in a droplet, and any suitable particle to which oligonucleotide tags can associate with (e.g., physically or chemically) may be used. The exact form of the particle is not critical. The particle may be spherical or non-spherical, and may be formed of any suitable material. In some cases, a plurality of particles is used, which have substantially the same composition and/or substantially the same average diameter. The “average diameter” of a plurality or series of particles is the arithmetic average of the average diameters of each of the particles. Those of ordinary skill in the art will be able to determine the average diameter (or other characteristic dimension) of a plurality or series of particles, for example, using laser light scattering, microscopic examination, or other known techniques. The average diameter of a single particle, in a non-spherical particle, is the diameter of a perfect sphere having the same volume as the non-spherical particle. The average diameter of a particle (and/or of a plurality or series of particles) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases.

Tags such as those described herein may be used in a variety of situations, for example, to screen or bioprospect for new drugs or other treatments, as a high-throughput screening technique, to study the effect various exposure conditions have on cells, or the like. In addition, it should be understood that the invention is not limited to only cell studies. For example, a chemical contained within a droplet may be exposed to a variety of different conditions (e.g., potential reactants or catalysts, reaction conditions, initiators, etc.) and the different conditions tagged for determining or optimizing the reactions that the chemical is able to participate in. In some cases, the chemical may be a biologically active chemical (e.g., a drug, a protein, a polymer, a carbohydrate, etc.), although in other cases, the chemical need not be biologically relevant. For example, the chemical may be a monomer or a polymer, a catalyst, a semiconductor material, etc.

As a non-limiting example, in one set of embodiments, a plurality of substantially identical cells can be exposed to a variety of substances (e.g., other cells, chemical compounds, soil samples, naturally-occurring samples, etc.), optionally under various physical conditions (e.g., various temperatures), to determine whether any of the substances have a beneficial or a detrimental effect on the cells. For example, the substantially identical cells may be cancer cells that are screened against a panel of substances to identify those substances that, alone or in combination, have anti-cancer or anti-tumor properties, or the substantially identical cells may be immune or other cells to which a certain activity is desired. The panel may include, for example, naturally-occurring compounds, synthetic compounds, compounds from a library, compounds sharing certain properties, etc. In some cases, the compounds may also be present at more than one concentration. Each member of the panel may be tagged as discussed herein, such that, upon exposure of a cell in a droplet (or other entity) to a panel member, an appropriate corresponding tag is added. The tags may also be joined together. Thus, for example, the substantially identical cells may be exposed to a variety of substances, in various combinations, with subsequent sorting of live cells from dead cells; the tags from the droplets containing the live cells (and/or the dead cells) may be separated and sequenced as discussed herein to determine those conditions or panel members which were able to kill the cells. In addition, the invention is not limited to only substantially identical cells. For example, in another set of embodiments, droplets containing different cells (or other species, e.g., chemicals) may be used.

In some aspects of the invention, cells may be contained within gels to protect the cells during manipulation of the cells. For example, one or more cells may be contained within gel particles, which can then be exposed to various conditions and/or tags, as discussed herein. Thus, for example, a cell contained within a gel may be exposed to a first condition, a second condition, etc., and one or more nucleic acids encoding the conditions may be associated with the gel. As a non-limiting example, a gel containing a cell may be contained within a microfluidic droplet such as discussed herein, and the microfluidic droplet may then be exposed to a variety of conditions, along with nucleic acids encoding such conditions. In some embodiments, additional agents may also be added to the gels. For example, magnetic particles may be added to some or all of the gels, e.g., to facilitate later sorting. As another example, fluorescent agents may be added to some or all of the gels, e.g., to facilitate identification of certain cells (or other species to be determined) that are contained within the gels.

As a non-limiting example, in one set of embodiments, one or more cells may be contained within a gel particle, which may be contained within a droplet. The droplet (containing the gel particle) may be exposed to a variety of conditions (for example, a species such as a drug), and each time the droplet is exposed to a condition, a nucleic acid encoding the condition is added to the droplet. In some cases, after exposure, a condition of the cells may be determined (for example sorting of live cells from dead cells), and in some cases, the sorting may be facilitated using microfluidic techniques, FACS, magnetic sorting (e.g., using magnetic beads), centrifugation, or other sorting or separation techniques known to those of ordinary skill in the art. In one set of embodiments, the gel is an agarose gel. Other examples of gels include acrylamide-based gels, such as polyacrylamide or poly N-isopropylpolyacrylamide, or hydrogels such as alginic acid that can be gelled by the addition of gelation initiators (for instance, ammonium persulfate and TEMED for acrylamide, or Ca²⁺ for alginate, etc.). Other examples of gels encapsulating cells can be seen in Int. Pat. Apl. Pub. No. WO 2008/109176, incorporated herein by reference.

In some embodiments, the gel may be one that is low-melt, e.g., the agarose may melt at temperatures below about 100° C., below about 80° C., below about 70° C., or below about 60° C. in some cases. In some cases, the gel may be chosen to be able to solidify upon exposure to relatively low temperatures, e.g., below about 60° C., below about 50° C., below about 40° C., below about 35° C., below 30° C., below about 25° C., or below about 20° C.

In some embodiments, as non-limiting examples, the average diameter of the gel may be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the gel may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. Combinations of these are also possible, e.g., the gel may have an average diameter of between about 25 micrometers and about 100 micrometers. In some embodiments, a gel may be prepared by containing a cell (or other suitable species) within a microfluidic droplet with a gel precursor. For example, the gel may contain agarose which can form a gel upon exposure to a suitable temperature, or some gels may be prepared by containing a precursor such as disclosed herein and exposing the precursor to a suitable gelation initiator.

Additional details regarding systems and methods for manipulating droplets in a microfluidic system follow, e.g., for determining droplets (or species within droplets), sorting droplets, etc. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

In certain embodiments of the invention, sensors are provided that can sense and/or determine one or more characteristics of the fluidic droplets, and/or a characteristic of a portion of the fluidic system containing the fluidic droplet (e.g., the liquid surrounding the fluidic droplet) in such a manner as to allow the determination of one or more characteristics of the fluidic droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like.

In some cases, the sensor may be connected to a processor, which in turn, cause an operation to be performed on the fluidic droplet, for example, by sorting the droplet, adding or removing electric charge from the droplet, fusing the droplet with another droplet, splitting the droplet, causing mixing to occur within the droplet, etc., for example, as previously described. For instance, in response to a sensor measurement of a fluidic droplet, a processor may cause the fluidic droplet to be split, merged with a second fluidic droplet, etc.

One or more sensors and/or processors may be positioned to be in sensing communication with the fluidic droplet. “Sensing communication,” as used herein, means that the sensor may be positioned anywhere such that the fluidic droplet within the fluidic system (e.g., within a channel), and/or a portion of the fluidic system containing the fluidic droplet may be sensed and/or determined in some fashion. For example, the sensor may be in sensing communication with the fluidic droplet and/or the portion of the fluidic system containing the fluidic droplet fluidly, optically or visually, thermally, pneumatically, electronically, or the like. The sensor can be positioned proximate the fluidic system, for example, embedded within or integrally connected to a wall of a channel, or positioned separately from the fluidic system but with physical, electrical, and/or optical communication with the fluidic system so as to be able to sense and/or determine the fluidic droplet and/or a portion of the fluidic system containing the fluidic droplet (e.g., a channel or a microchannel, a liquid containing the fluidic droplet, etc.). For example, a sensor may be free of any physical connection with a channel containing a droplet, but may be positioned so as to detect electromagnetic radiation arising from the droplet or the fluidic system, such as infrared, ultraviolet, or visible light. The electromagnetic radiation may be produced by the droplet, and/or may arise from other portions of the fluidic system (or externally of the fluidic system) and interact with the fluidic droplet and/or the portion of the fluidic system containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. As an example, a laser may be directed towards the fluidic droplet and/or the liquid surrounding the fluidic droplet, and the fluorescence of the fluidic droplet and/or the surrounding liquid may be determined. “Sensing communication,” as used herein may also be direct or indirect. As an example, light from the fluidic droplet may be directed to a sensor, or directed first through a fiber optic system, a waveguide, etc., before being directed to a sensor.

Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

As used herein, a “processor” or a “microprocessor” is any component or device able to receive a signal from one or more sensors, store the signal, and/or direct one or more responses (e.g., as described above), for example, by using a mathematical formula or an electronic or computational circuit. The signal may be any suitable signal indicative of the environmental factor determined by the sensor, for example a pneumatic signal, an electronic signal, an optical signal, a mechanical signal, etc.

In one set of embodiments, a fluidic droplet may be directed by creating an electric charge and/or an electric dipole on the droplet, and steering the droplet using an applied electric field, which may be an AC field, a DC field, etc. As an example, an electric field may be selectively applied and removed (or a different electric field may be applied, e.g., a reversed electric field) as needed to direct the fluidic droplet to a particular region. The electric field may be selectively applied and removed as needed, in some embodiments, without substantially altering the flow of the liquid containing the fluidic droplet. For example, a liquid may flow on a substantially steady-state basis (i.e., the average flowrate of the liquid containing the fluidic droplet deviates by less than 20% or less than 15% of the steady-state flow or the expected value of the flow of liquid with respect to time, and in some cases, the average flowrate may deviate less than 10% or less than 5%) or other predetermined basis through a fluidic system of the invention (e.g., through a channel or a microchannel), and fluidic droplets contained within the liquid may be directed to various regions, e.g., using an electric field, without substantially altering the flow of the liquid through the fluidic system.

In some embodiments, the fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc.

In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. The liquid reservoirs may be positioned such that, when activated, the movement of liquid caused by the activated reservoirs causes the liquid to flow in a preferred direction, carrying the fluidic droplet in that preferred direction. For instance, the expansion of a liquid reservoir may cause a flow of liquid towards the reservoir, while the contraction of a liquid reservoir may cause a flow of liquid away from the reservoir. In some cases, the expansion and/or contraction of the liquid reservoir may be combined with other flow-controlling devices and methods, e.g., as described herein. Non-limiting examples of devices able to cause the expansion and/or contraction of a liquid reservoir include pistons and piezoelectric components. In some cases, piezoelectric components may be particularly useful due to their relatively rapid response times, e.g., in response to an electrical signal. In some embodiments, the fluidic droplets may be sorted into more than two channels.

As mentioned, certain embodiments are generally directed to systems and methods for sorting fluidic droplets in a liquid, and in some cases, at relatively high rates. For example, a property of a droplet may be sensed and/or determined in some fashion (e.g., as further described herein), then the droplet may be directed towards a particular region of the device, such as a microfluidic channel, for example, for sorting purposes. In some cases, high sorting speeds may be achievable using certain systems and methods of the invention. For instance, at least about 10 droplets per second may be determined and/or sorted in some cases, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1,000 droplets per second, at least about 1,500 droplets per second, at least about 2,000 droplets per second, at least about 3,000 droplets per second, at least about 5,000 droplets per second, at least about 7,500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be determined and/or sorted.

In some aspects, a population of relatively small droplets may be used. In certain embodiments, as non-limiting examples, the average diameter of the droplets may be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. Combinations of these are also possible, e.g., the droplet may have an average diameter of between about 25 micrometers and about 100 micrometers. The “average diameter” of a population of droplets is the arithmetic average of the diameters of the droplets.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., the droplets may have a distribution of diameters such that no more than about 5%, no more than about 2%, or no more than about 1% of the droplets have a diameter less than about 90% (or less than about 95%, or less than about 99%) and/or greater than about 110% (or greater than about 105%, or greater than about 101%) of the overall average diameter of the plurality of droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link et al., published as WO 2004/091763 on Oct. 28, 2004, incorporated herein by reference.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Certain embodiments are generally directed to systems and methods for splitting a droplet into two or more droplets. For example, a droplet can be split using an applied electric field. The droplet may have a greater electrical conductivity than the surrounding liquid, and, in some cases, the droplet may be neutrally charged. In certain embodiments, in an applied electric field, electric charge may be urged to migrate from the interior of the droplet to the surface to be distributed thereon, which may thereby cancel the electric field experienced in the interior of the droplet. In some embodiments, the electric charge on the surface of the droplet may also experience a force due to the applied electric field, which causes charges having opposite polarities to migrate in opposite directions. The charge migration may, in some cases, cause the drop to be pulled apart into two separate droplets.

Some embodiments of the invention generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Thus, in certain embodiments, the design and/or fabrication of the article may be relatively simple, e.g., by using relatively well-known soft lithography and other techniques such as those described herein. In addition, in some embodiments, rapid and/or customized design of the article is possible, for example, in terms of geometry. In one set of embodiments, the article may be produced to be disposable, for example, in embodiments where the article is used with substances that are radioactive, toxic, poisonous, reactive, biohazardous, etc., and/or where the profile of the substance (e.g., the toxicology profile, the radioactivity profile, etc.) is unknown. Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

The following documents are incorporated herein by reference in their entirety for all purposes: Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al. In addition, U.S. Provisional Patent Application Ser. No. 61/981,123, filed Apr. 17, 2014, entitled “Systems and Methods for Droplet Tagging,” by Nicol, et al., and U.S. Provisional Patent Application Ser. No. 61/981,108, filed Apr. 17, 2014, entitled “Methods and Systems for Droplet Tagging and Amplification,” by Weitz, et al. are each incorporated herein by reference in its entirety.

In addition, the following are incorporated herein by reference in their entireties: U.S. Pat. Apl. Ser. No. 61/981,123 filed Apr. 17, 2014; a PCT application filed Apr. 17, 2015, entitled “Systems and Methods for Droplet Tagging”; U.S. Pat. Apl. Ser. No. 61/981,108 filed Apr. 17, 2014; a U.S. patent application filed on Apr. 17, 2015, entitled “Immobilization-Based Systems and Methods for Genetic Analysis and Other Applications”; a U.S. patent application filed on Apr. 17, 2015, entitled “Barcoding Systems and Methods for Gene Sequencing and Other Applications”; U.S. Pat. Apl. Ser. No. 62/072,944, filed Oct. 30, 2014; and a PCT application filed on Apr. 17, 2015, entitled “Systems and Methods for Barcoding Nucleic Acids.”

The following examples are intended to illustrate certain embodiments of the present invention, but do not exemplify the full scope of the invention.

### Example 1

High-throughput screening (HTS) is a method for drug discovery. Using robotics, data processing and control software, liquid handling devices, and sensitive detectors, a researcher can conduct large scale of pharmacological tests, or identify active compounds, antibodies, or genes that modulate a particular biomolecular pathway. The key labware of HTS is the microtiter plate, which can have, e.g., 384, 1536, or 3456 wells, and current robots can often test up to 100,000 compounds per day. However, this technology is approaching its physical limit; below the 1-microliter-volumes of 1,536-well plates, evaporation and capillary forces become significant.

Developments on microwell-based microfluidic technology have significantly improved screening capabilities, increased the speed by 10-fold and decreased the reaction volume by 1,000-fold. The use of water-in-oil drops eliminates solid wells used in microtiter plates; this can simplify engineering and/or expand the capacity of drug screening within an acceptable time and cost scale. This is demonstrated in this example, where droplets are multi-functionalizd to demonstrate drug screening with a high level of combinations, e.g., to test their synergistic effects on cells.

To construct massive drug combinations, three groups of relatively monodisperse picoliter drops were first individually generated in 96-parallel microfluidic drop-makers. Each group included 96 kinds of drops with different drugs and their different concentrations, along with a unique pre-mixed oligonucleotide index in the solution (96 was used here as an illustrative example, although other numbers of drops could have been used in other embodiments). These three groups of drops were then merged using a microfluidic drop-merger in a random combination of different drugs and different concentrations. Single K562 chronic myeloid leukemia cells were introduced to the drug combinations by picoinjecting a cell suspension to the merged drops at a concentration known to obtain a Poisson distribution with rate λ (lambda)=0.1, and incubated at 37° C. for 24 hours. See, e.g., U.S. Pat. Apl. Pub. No. 2012/0132288, entitled “Fluid Injection,” incorporated herein by reference in its entirety.

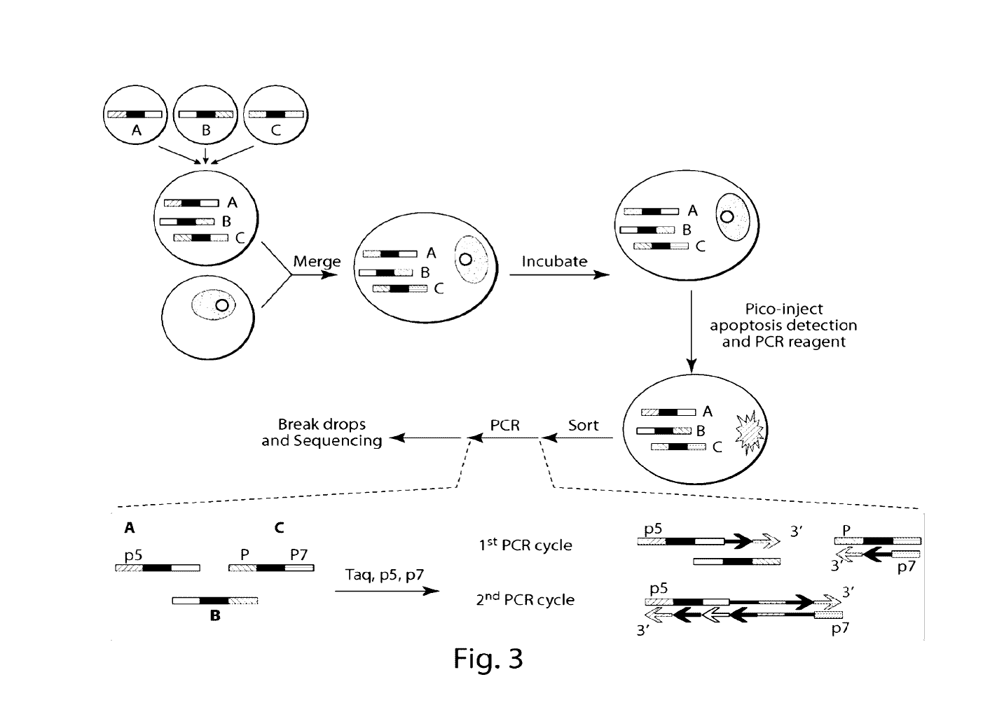

By adding a fluorogenic substrate, caspalux6-J1D2, which is specifically cleaved by increased caspase 3 and caspase 3-like activities during apoptosis, apoptosis of cells in drops can be determined. In this apoptosis assay solution, a PCR cocktail was included to link the oligonucleotide indexes to a full-length double-stranded DNA barcode through PCR amplification. After incubation at 37° C. for half an hour, the drops containing apoptotic cells that suggest effective drug combinations were sorted according to fluorescence intensity, followed by PCR amplification and next-generation sequencing (NGS) to decode the double-stranded DNA barcodes in each sorted drops, which were used to reveal the optimal drug combinations. The schema of this large-scale drug combination screening system is shown in FIG. 3, showing large-scale drug combination screening in drop-based microfluidics.

The strategy to create a double-stranded DNA “barcode” representing three oligos/three drugs and their different concentrations is presented in the FIG. 3 inset. To form this DNA barcode, three families of oligonucleotide indexes are used, a left oligonucleotide (A), a center oligonucleotide (B) and a right oligonucleotide (C). The left (A) and center (B) partially overlap, and the center (B) and right (C) partially overlap. These overlaps allow the three oligonucleotides to anneal to each other when they are present in a single drop, as discussed below. The drug defining unique barcode is encoded in the non-overlapping parts of the left, center and right oligonucleotides. After two rounds of PCR, these three oligonucleotides result in a double stranded “ABC” DNA “barcode.” To allow the DNA barcode to be sequenced through NGS, common sequencing primers P5 and P7 are integrated on the 5′ end of the left (A) oligonucleotide and the 3′ end of the right (C) oligonucleotide, respectively. The annealing and PCR are performed within individual droplets, e.g., to make sure the 3 barcodes are linked together to allow subsequent sequence analysis to reveal what 3 drugs were combined based on the oligonucleotides within the “barcode.” A bioinformatics pipeline to decode the DNA barcodes from NGS reads has been developed.

An even annealing and amplification of combinations of four A, four B and four C oligos in bulk, 64 barcode combinations in total, is shown in FIG. 4; this property allows for quantitatively analyzing how many cells have been induced to undergo apoptosis by counting the unique barcode reads. Furthermore, another advantage for this drop-based platform to perform quantitative apoptosis detection is that the loss of apoptotic cells was minimized compared with bulk assays, in which several staining and washing steps diminish the accuracy for apoptosis detection.

FIG. 4 shows even amplification of 64 barcode combinations in bulk decoded by deep sequencing, representing three oligonucleotides/three drugs and their different concentration combinations. It should be noted that each “barcode” was amplified by substantially the same amount, i.e., the amplification was “even,” e.g., rather than favoring one or two barcodes at the expense of the other barcodes.

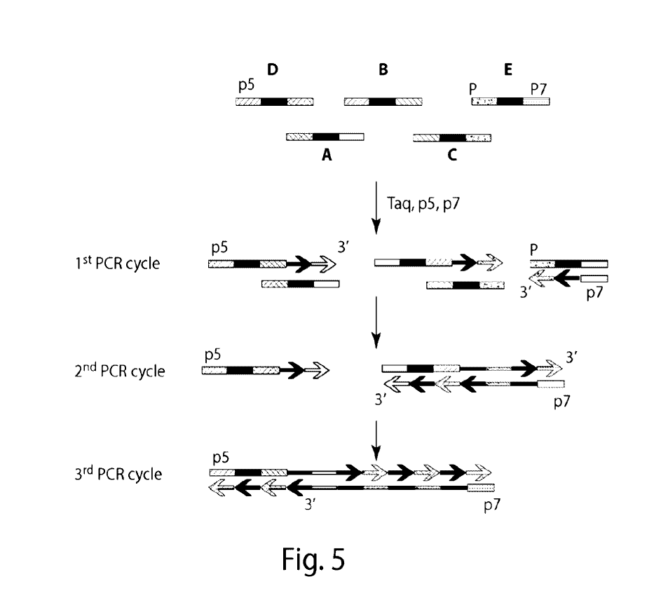

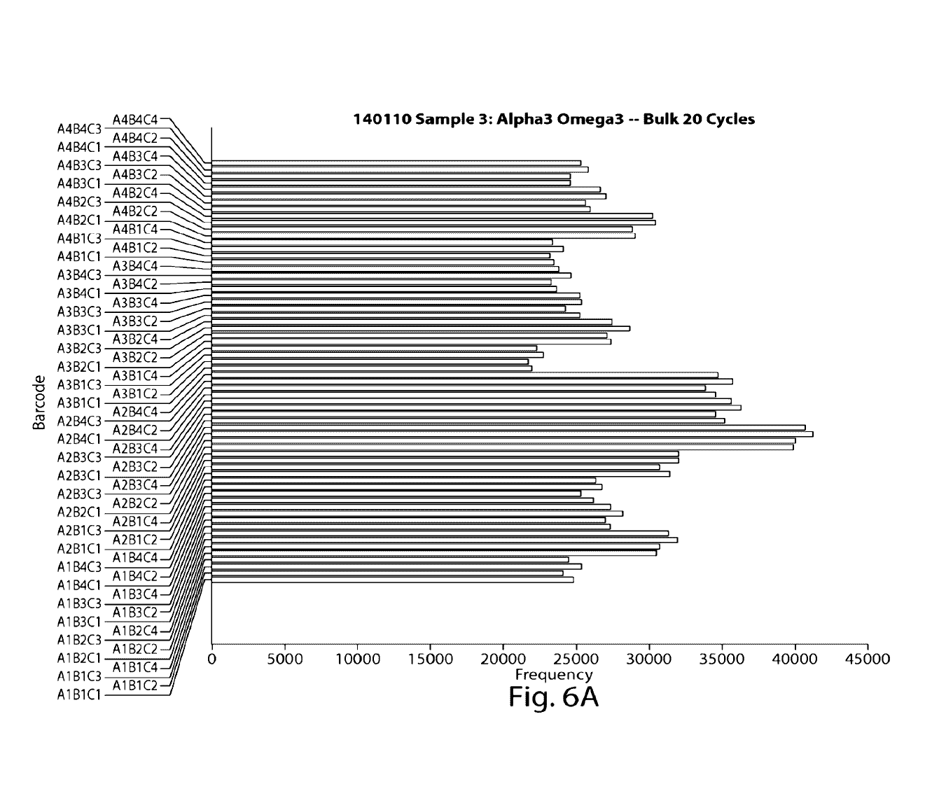

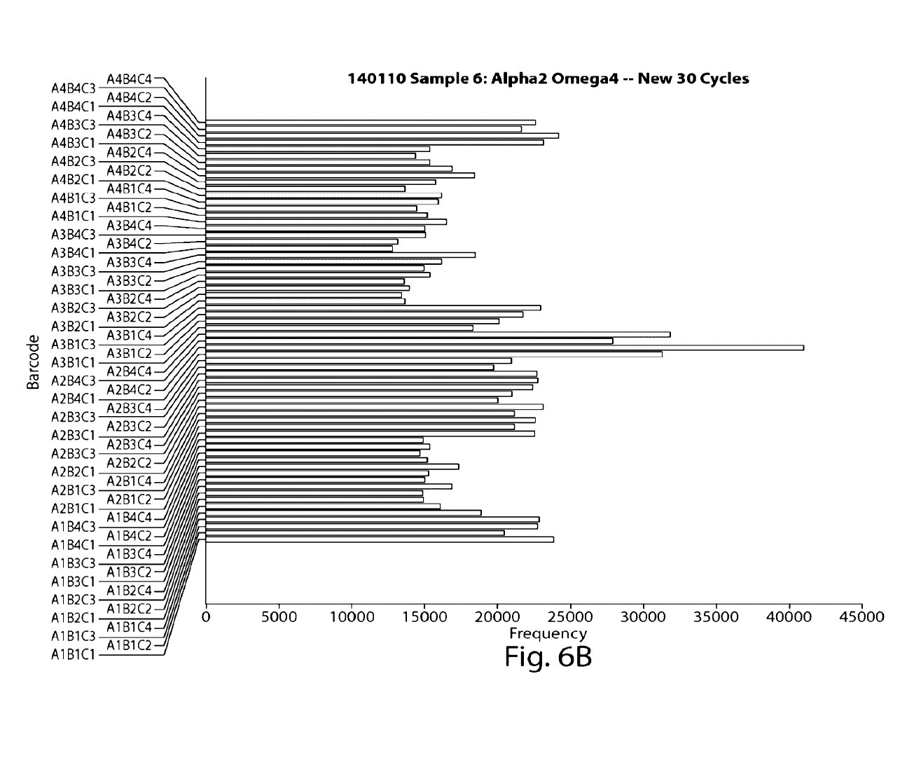

Given that each group of drops had at most 96 kinds of drops with different drugs and their different concentrations, and there are a total of three groups of drops, nearly 1 million drug combinations could be obtained (96×96×96). To further scale up the screening in terms of multiple cell lines without increasing the deep-sequencing run, two oligonucleotide indexes D and E were added into the solution containing the PCR cocktail in another set of experiments. The formation of double stranded DNA barcodes shared a similar mechanism as that described above. Briefly, the newly added oligo indexes D and E integrate P5 had P7 sequences, and partially overlap with oligo A and C, respectively. Instead of in two cycles of PCR, the final barcode DNA was constructed in three cycles of PCR, as shown in FIG. 5, showing a strategy to create a double-stranded DNA barcode combining three oligonucleotide tagging drugs and two more barcodes tagging the cell lines. Even amplifications of 64 barcode combinations with two more barcodes to tag the cell lines is shown in both bulk and drop-based amplification (FIG. 6). This strategy allowed screening of drug combinations and cell lines in a high-throughput and cost- and time-effective way in this example.

FIG. 6 shows even amplification of 64 barcode combinations with 2 more barcodes to tag the samples is shown by deep sequencing. FIG. 6A shows bulk amplification, while FIG. 6B shows drop-based amplification.

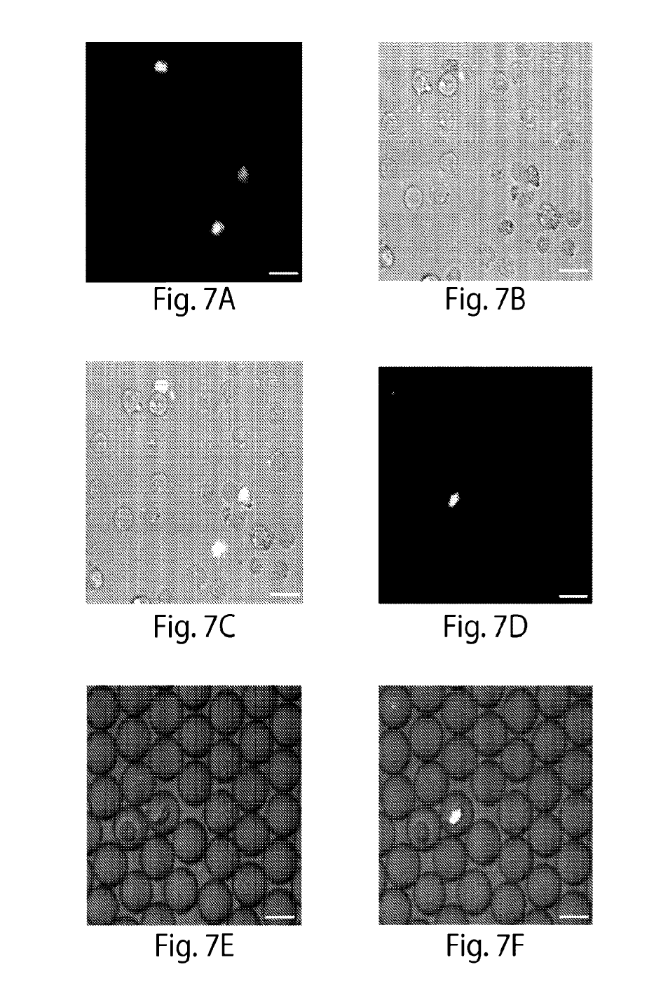

In this example, apoptosis was selected as an evaluation marker for drug combination efficiency; this is because apoptosis is deregulated in many cancers, making it difficult to kill tumors, and drugs that restore the normal apoptotic pathways have the potential for effectively treating cancers that depend on aberrations of the apoptotic pathway to stay alive. However, in other cases, other evaluation markers could also have been studied. Since in drop-based apoptosis detection, the dye is not washed out, a wash-free dye was used here, which not only distinctively stains apoptotic cells, but shows sufficient contrast between cells and background. Caspalux6-J1D2 met these requirements, and showed a clear apoptotic signal in the Imatinib-treated K562 cells in both bulk and drop experiment; this result is shown in FIG. 7.

FIG. 7 shows apoptosis detection of K562 cells in bulk and drop experiment after being treated with Imatinib for 24 hours. FIGS. 7A-7C, bulk experiment: fluorescence field (FIG. 7A), bright field (FIG. 7B), and merging (FIG. 7C). FIGS. 7D-7F, drop experiment: fluorescence field (FIG. 7D), bright field (FIG. 7E), and merging (FIG. 7F).

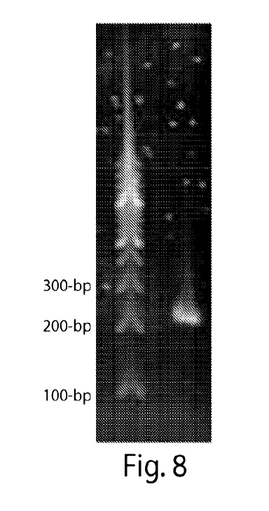

One consideration about the dye used is that it potentially could inhibit the amplification and the construction of the double-stranded DNA barcode. In these examples, full-length DNA barcodes was amplified with the presence of an apoptosis assay reagent, as shown is FIG. 8, thus showing that this is less of a concern. FIG. 8 shows successful amplification of 64 barcode combinations with two more barcodes in drops in the presence of apoptosis assay reagent.

These examples describe a drop-based microfluidic system for quantitative drug combination screening. Although the current example describes a three-group oligo merging, the assay can also be updated to allow for five, seven or more combinations of oligonucleotides in single droplets. This assay screens for cell killing using a fluorescent apoptosis signal, but can also work for other biomarkers. Furthermore, the assay may work for any biologically active agent to be screened not just drugs, but other agents such as siRNA, shRNA, TALENs, CRISPR-Cas and retroviral libraries, etc.

### Example 2

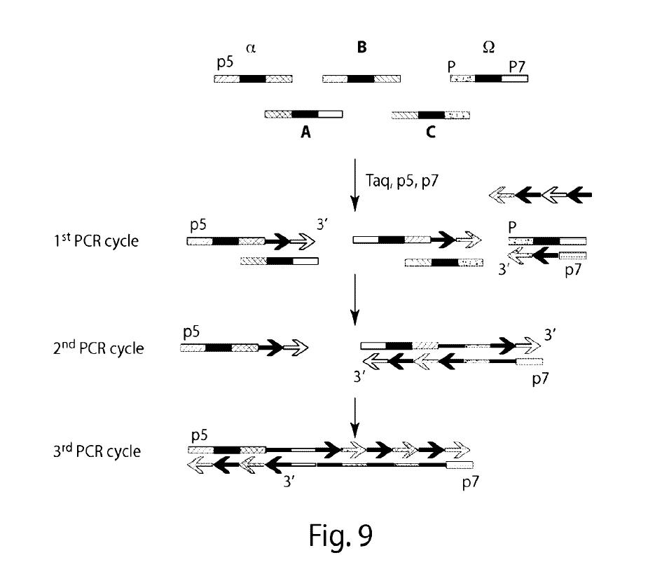

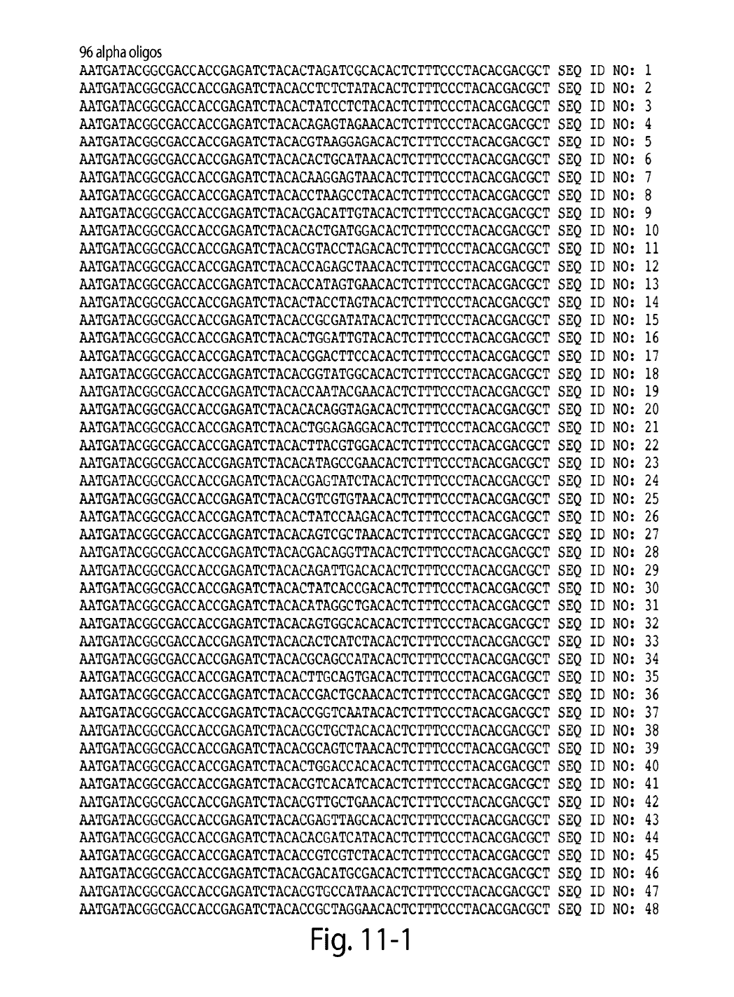

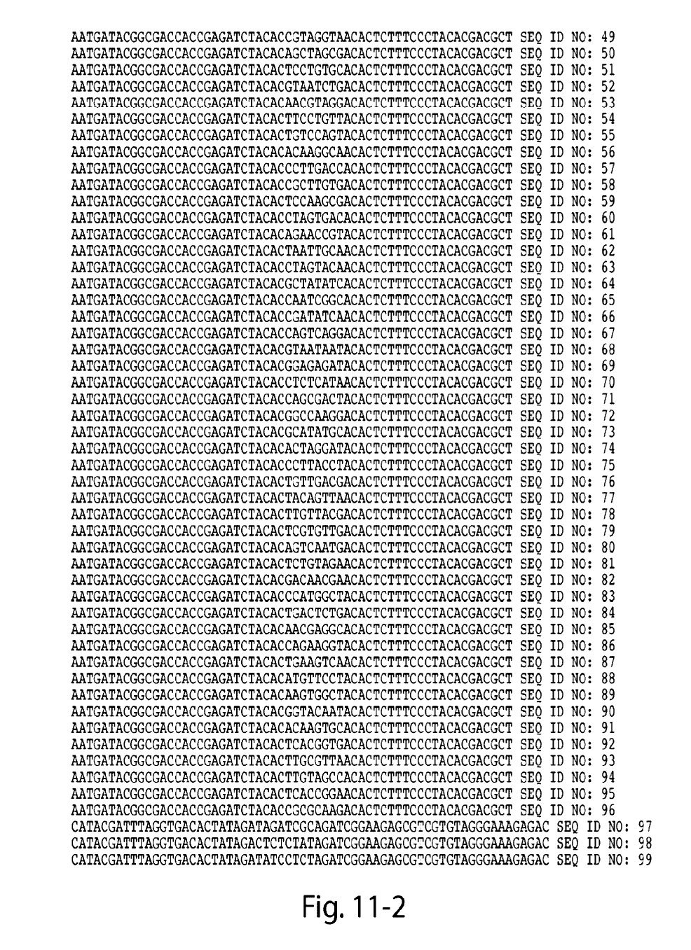

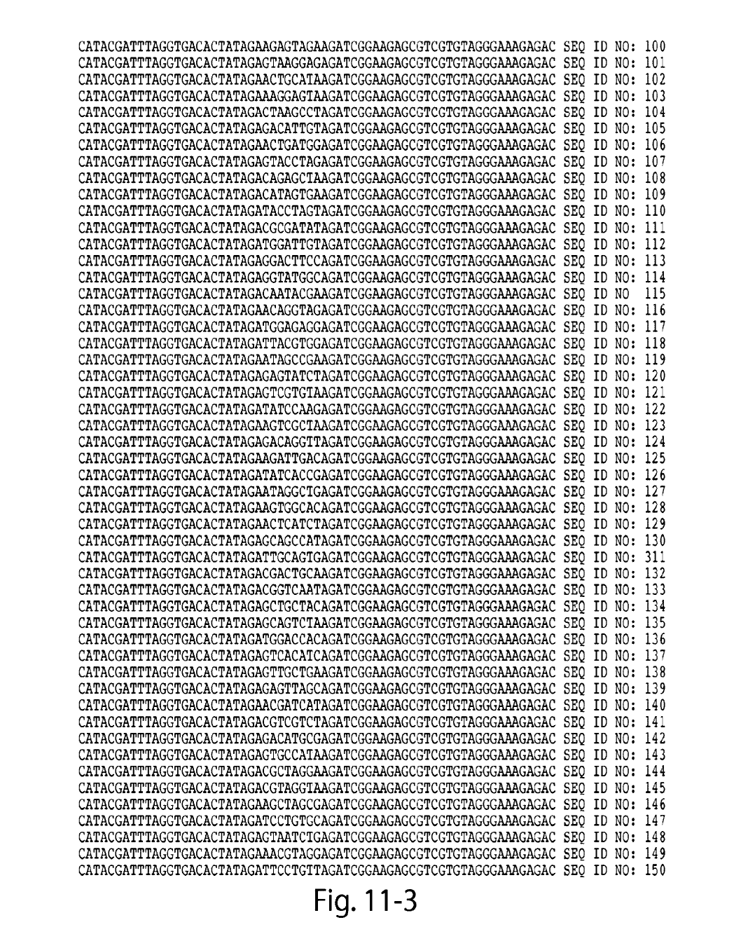

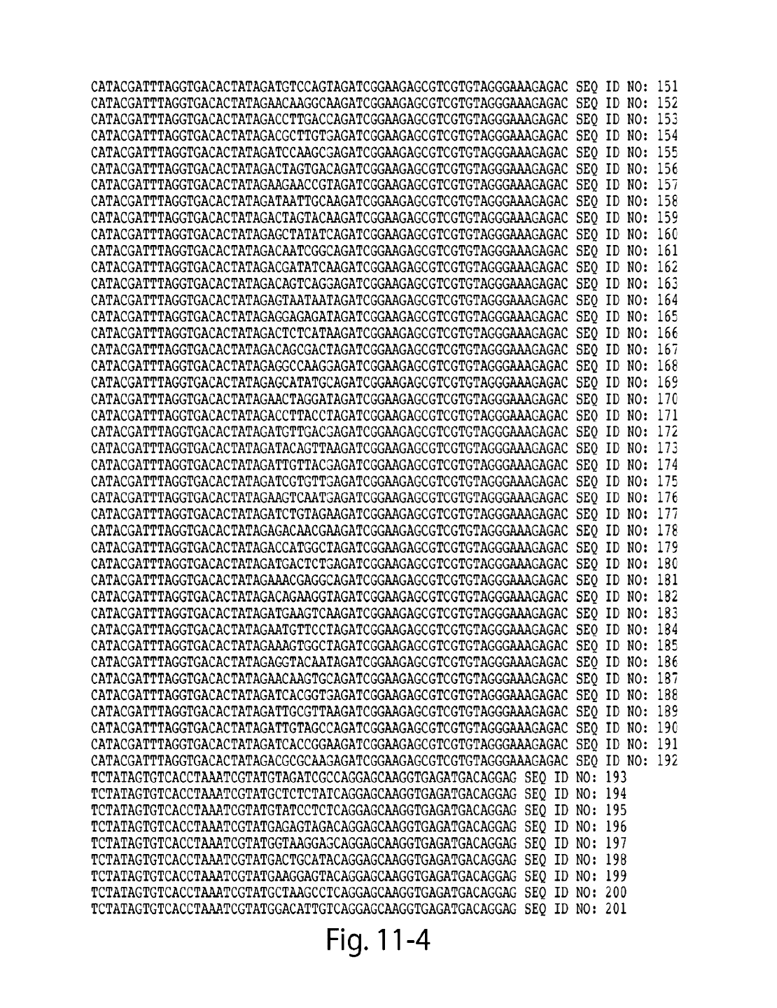

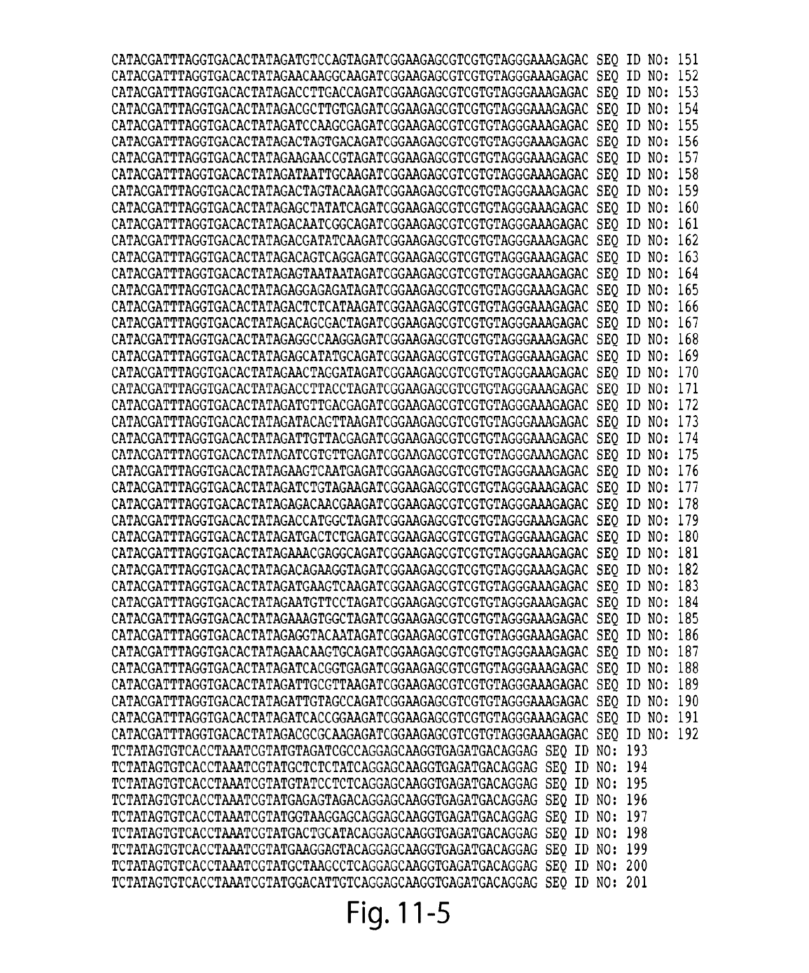

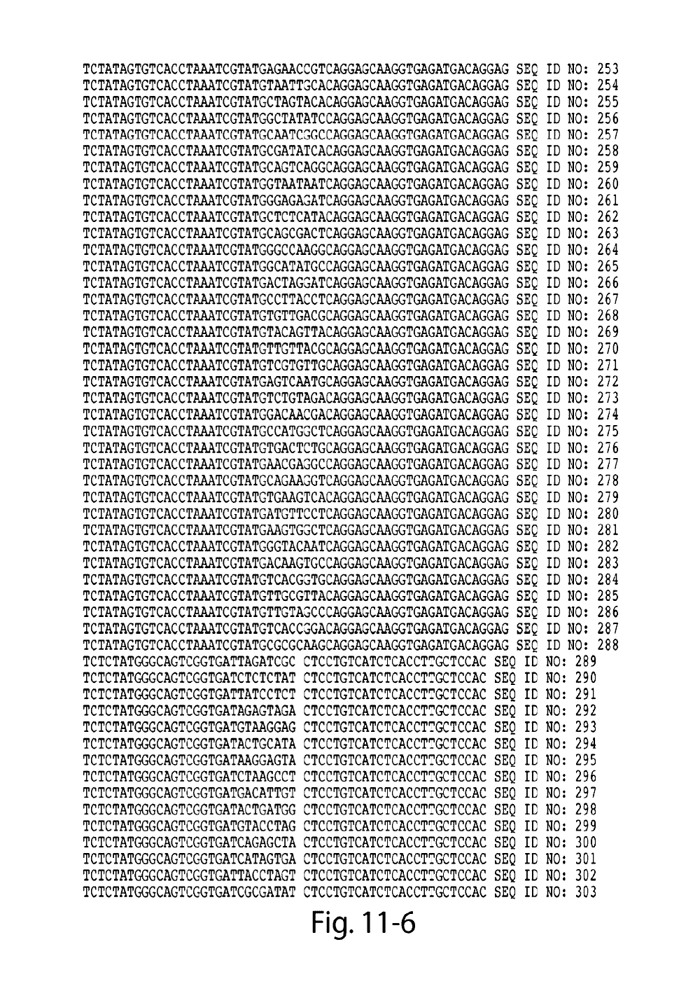

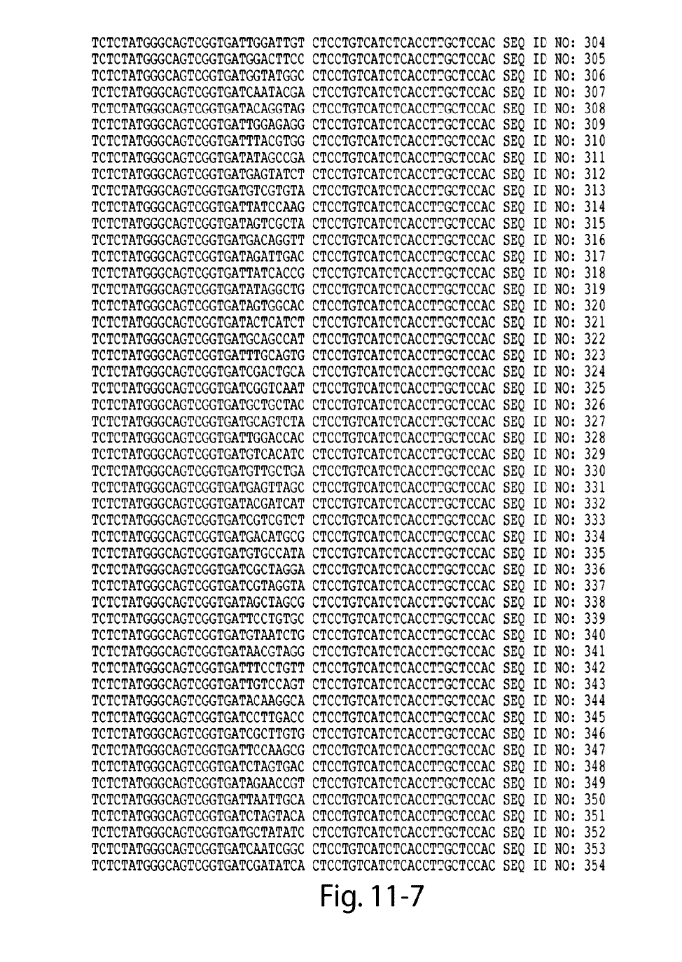

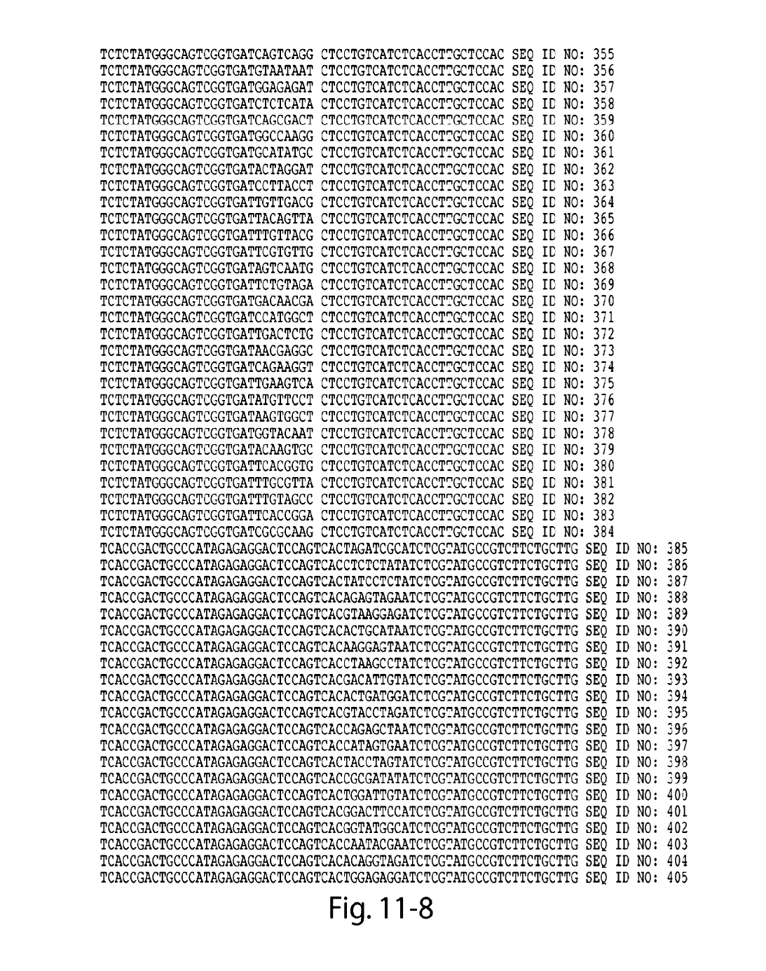

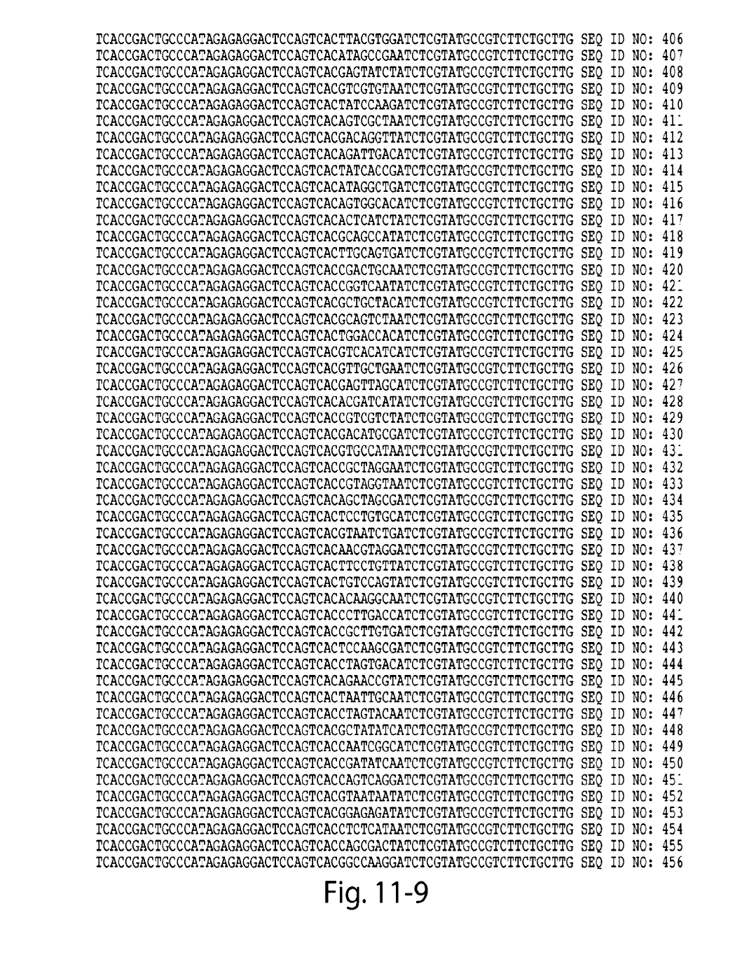

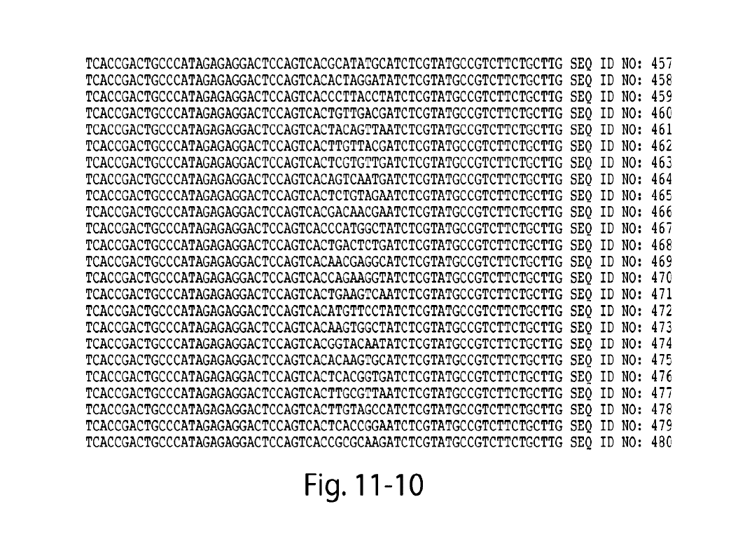

This example illustrates the design of drug-barcoding method by an example of testing 3-drug interaction from a set of 24 drugs, each with 4 concentrations (0, IC25, IC50 and IC75). 96 different oligonucleotides were designed that contain different barcodes for labeling 96 (=24×4) drug-concentrations. Every oligonucleotide contained linker sequences flanking both sides of its barcode. To test up to 3 drug interactions, 3 different pairs of linkers (A, B, and C) were used to generate 3 sets of oligonucleotides for labeling the same 96 drug-concentrations 3 times, redundantly. The linkers allowed for linker-linker connection via PCR to yield A-B-C combinations (total n=96×96×96), and for addition of downstream sequencing adaptor primers (denoted as α, alpha and Ω, omega), resulting in full-length α-A-B-C-Ω PCR products that were ready for sequencing (FIG. 9). 96 different alpha oligonucleotides were used for barcoding experiment conditions such as incubation time, technical replications etc. 96 different omega oligonucleotides for barcoding cell lines to be tested could have also be used. 8-nt random mers were used here as barcodes, although these could have also been shorter or longer. Alpha and omega were designed for, but not limited to, the Illumina Miseq platform. FIG. 11 lists oligonucleotide sequences for alpha, A, B, C and omega, each with 96 different barcodes (480 sequences). The full-length of the oligonucleotide barcode PCR product was 212 base pairs, among which only 8, 74, and 8 base pairs need to be sequenced and contain barcode for alpha, A-B-C, and omega, respectively. This simple non-limiting example is capable of barcoding nearly 1 billion (n=96×96×96) “drug-concentration/experiment condition/cell line” combinations, with a capacity of up to 3 drug interactions. The 96 barcodes could also be used for testing 48 drugs with 2 concentrations. Further, this example is easily scaled up to higher levels of drug interactions (5 or 10 if necessary) by increasing the number of replicate plates and higher number of testing drugs by using more oligo barcodes (for example, 8-nt random mers could provide 65,536 possible barcodes).

FIG. 9 shows the strategy used in this example to create a double-stranded DNA barcode combining three oligonucelotides (A, B, and C) tagging drugs and two more barcodes (alpha and omega) tagging the cell lines.

Fabrication of microfluidics devices. Polydimethylsiloxane (PDMS) devices were fabricated using replica molding with SU8 photo resist as the mold master. The PDMS devices were rendered more hydrophobic by coating them with Aquapel (Rider, MA, USA). Aquapel was injected into the devices and then the devices were dried by blowing air into them and baking at 65° C. for 15 minutes. Electrodes were fabricated on chip using low melting temperature solder.

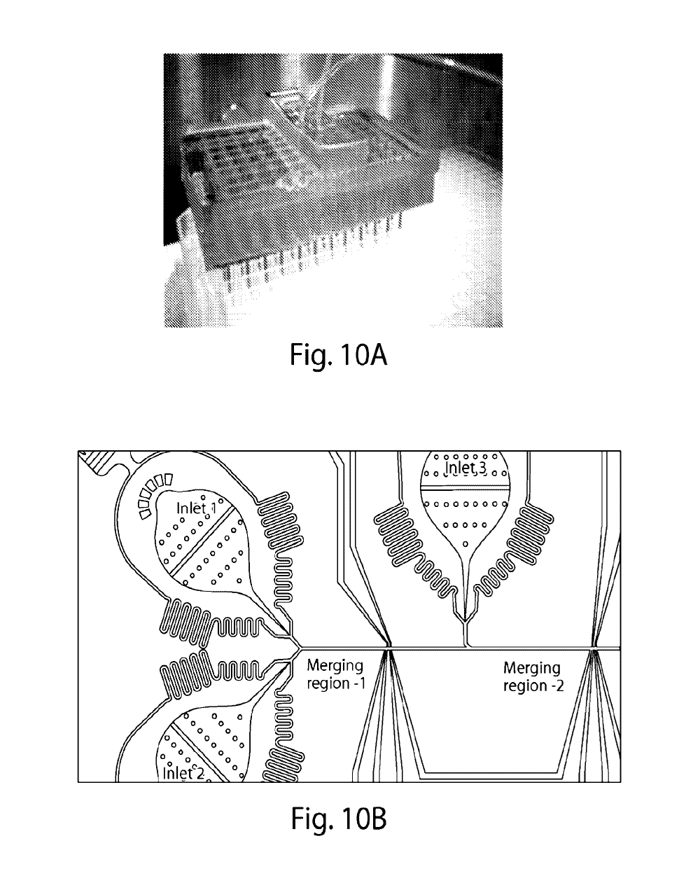

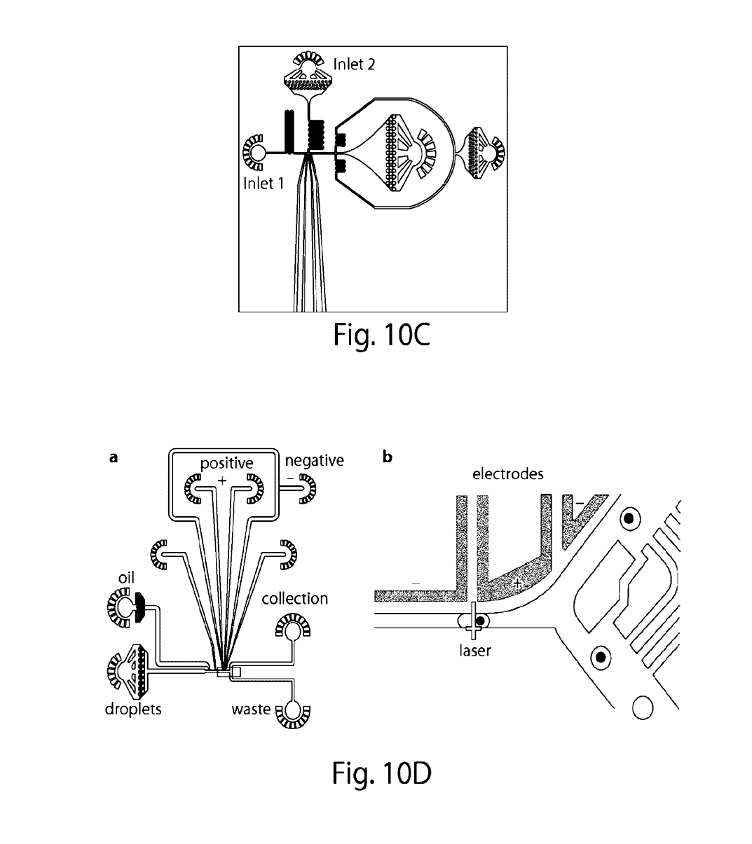

Encapsulation of the oligo-drug library emulsion. Three 96-well plates (A, B and C), each contained 24 different drugs with 4 different concentrations, thus these experiments used 96 drug-concentrations per plate, 3 replicate plates. To plate A, 96 different A oligonucleotides were added. Similarly, 96 B oligonucleotides and 96 C oligonucleotides are added to plate B and C, respectively. To encapsulate the oligonucleotides in drops, 96 parallel drop-makers were designed on a single microfluidic chip, so that the aqueous inlets of each drop-maker (22 gauge stainless steel capillaries, New England Small Tube) fit one quarter of a 384 well-plate and were immersed in 96 different wells of a 96-well plate, each containing a unique drug at certain concentration and a unique oligonucleotide. See FIG. 10A.

Oil with 1% w/w surfactant was distributed to all drop-makers via a common inlet that was connected to a pressurized oil reservoir. The plate and the microfluidic parallel device were placed in a pressure chamber while a common outlet for all 96 drop-makers was located outside the pressure chamber. Upon pressurizing the chamber, each of the 96 oligonucleotide-drug solutions was forced through its own drop-maker, thereby forming an emulsion at junction of the oil and oligonucleotide-drug solution. The oil reservoir was pressurized to 9 psi (1 psi ˜6894,757 Pa) and the pressure chamber to 6 psi, producing ˜40 micrometer drops at a rate of about 500 microliters/min from 96 wells. This encapsulation process was performed two more times using two new 96 parallel drop-makers from the other two 96-well plates. These two drop-makers had a smaller junction size to produce ˜25 micrometer drops.

Generation of drug combination library emulsion. To obtain a random drug combination library of different drugs and different concentrations, a three-drop merging microfluidic chip was designed to merge the three groups of drops from the three 96-well plates (FIG. 10B). Large drops and one group of the small drops was injected to the inlet 1 and inlet 2; drops in each group were spaced by flowing in oil with 1% w/w surfactant. By adjusting the flow rates of drops and oil, the two groups of drops were synchronized to flow in the same channel. The small drops flowed faster than the large drops; this allowed the small drops to chase the large drops to form a pair. The droplets were then merged together when a pair of the large drop and small drop passed an electric field in the emerging region-1. The electric field was generated by the electrodes connected with a high-voltage amplifier (Trek). The other group of small drops were injected into the inlet 3, spaced by oil with 1% w/w surfactant as well. These drops were synchronized with the merged first two groups of drops and merge them at the merging region-2. Finally, the drops were collected at the outlet. Each successfully merged drop from these three groups of drops contained a random drug combination and an oligonucleotide combination of A, B, and C.

Pico-injection of target cells and incubation. The merged droplets were injected into inlet-1 of a microfluidic pico-injector, spaced by oil with 1% w/w surfactant. A K562 cell solution was prepared and injected into the inlet-s. When the electric filed was applied, the cell solution was injected to the drops containing drugs and oligonucleotides. By calculating the concentration of the cells, this resulted in around one cell per ten drops. To prevent sedimentation in the syringe of cells facing encapsulated, a custom-built, motorized cell stirring setup was used. A 1.5 mm×8 mm magnetic stirrer bar (VWR, USA) was placed in the syringe containing the cell solution. A small bar magnet was mounted on a DC gear motor (Firgelli Automations, USA) that rotates the magnet, thus reversing the polarity of the magnetic field in its vicinity. This external setup was placed close to the syringe containing the cells to be encapsulated. Continuous rotation of the motor resulted in the rotation of the magnetic field of the bar magnet mounted on the motor. This rotating magnetic field forces the magnetic stirrer bar inside the syringe to rotate continuously, thus stirring the cell solution. The drops were collected in a 1.5 ml Eppendorf tube with cap open, and the tube sealed with one thin strip of stretched Parafilm; this allowed efficient oxygen and CO₂ exchange, and simultaneously prevented evaporating. The drops were incubated at 37° C. in a carbon dioxide cell incubator for 24 h.

Pico-injection of apoptosis assay reagent and PCR cocktail. To add an apoptosis assay reagent to detect apoptotic cells, and to link the three oligos in one drop to a full-length barcode and amplify the linked barcode, apoptosis assay reagent and PCR cocktail was mixed together, and pico-injected into the incubated drops. The component of the mixture is listed in Table 1. Here, alpha and omega were used as a indexes for different samples; this allowed performance of deep-sequencing on different samples together, and further increased the throughput. Drops were collected at the outlet, and incubated at 37° C. for half an hour.

TABLE 1
Component of the mixture of apoptosis assay reagent and
PCR cocktail

| Alpha-Omega Master Mix | Volume (microliters) |
|---|---|
| Buffer for Taq, 10X | 24 |
| Mg²⁺, 50 mM | 7.2 |
| dNTP, 10 mM | 4.8 |
| P7, 10 micromolar | 6 |
| P5, 10 micromolar | 6 |
| α (alpha) #, 8.8 micromolar | 2.4 |
| Ω (omega) #, 8.8 micromolar | 2.4 |
| 10 mg/ml BSA | 4.8 |
| 10% Tween 20 | 4.8 |
| Platinum Taq Polymerase | 2.4 |
| 10 micromolar substrate solution | 48 |
| Fetal bovine serum (FCS) | 12 |
| TOTAL | 120 |

Detection and sorting of apoptotic cells and PCR. The incubated drops were injected into a sorting microfluidic device at a flow rate of 40 microliters/h and evenly spaced by HFE-7500 oil with 1% surfactant flowing (FIG. 10C). The detection and sorting region was aligned to a laser induced fluorescence system. When a drop passes by the laser, its fluorescence generated from an apoptotic cell is collected by a microscope objective and focused on a photomultiplier tube (Hammamatsu) which is connected to a custom computer LabView program running on a real-time field-programmable gate array card (National Instruments). All drops were gated based on detector peak width to exclude outliers, such as doublets and triplets. The counting of bright drops represents the number of apoptotic cells. Drops then flowed to the asymmetric “Y” sorting junction, where they can take one of two paths. In the absence of sorting, droplets preferentially flow to the waste channel, as it has a lower fluidic resistance. When a drop is bright enough to cross the voltage threshold set in the program, the software sends several cycles of a 20 kHz single-ended square wave to the sorting electrodes after being amplified by a factor of 1,000 by a high-voltage amplifier. The bright drops were sorted to 20 microliters of empty drops. Then, the drops were flowed and washed into a PCR tube, the surface covered with mineral oil, and the tube placed in a PCR machine. The thermocycling conditions were 95° C. for 5 min, 10 cycles of 95° C. for 30 sec and 60° C. for 2 min, 10 cycles of 95° C. 30 sec, and 60° C. for 45 sec.

FIG. 10 shows the design of the microfluidic device. FIG. 10A shows 96 drop-makers. FIG. 10B shows an example device for there-drop merging. FIG. 10C shows a pico-injector. FIG. 10D shows a microfluidic sorter (left, overall design; right, zoom-in at sorting junction).

Break the drops and prepare sequencing library. To break or burst the drops, 20% of 1H,1H,2H,2H-perfluorooctanol (PFO) (Alfa Aesar, Ward Hill, Mass.) was added, vortexed, and centrifuged for 5 min at 5,000 rpm, followed by a cleanup step using SPRI beads (1.0×) cleanup immediately and eluted in 20 microliters of nuclease-free water. To remove remaining single-strand oligonucelotides, the sample was treated with exonuclease (ExoSAP-IT, Affymetrix, CA) at 37° C. for 15 min, followed by 80° C. for 15 min to inactivate exonuclease. The samples were then subjected to a PCR amplification to yield full-length and sufficient library molecule for sequencing. The PCR reaction contained alpha and omega oligonucleotides. The thermocycling conditions were 95° C. for 5 min, 20 cycles of 95° C. for 30 sec, 60° C. for 30 sec, and 72° C. for 60 sec.

Deep sequencing. The library was quantified using a qPCR kit (KAPA Library Quantification Kit—Illumina, MA). A total of 12 billions of pooled library molecules from different experiment conditions were loaded on the Miseq sequencer and sequenced using a 300-cycles V2 kit according to the Nextera dual-index sequencing protocol.

Data analysis. Raw data in BCL format from the Miseq was converted to FASTQ format using the CASAVA v1.8.2 software from Illumina. A custom script was used to annotate alpha, A, B, C, and omega in each sequence read. Briefly, linker sequences were masked. An array of A barcodes (n=96) were queried in the position window of the read where A barcodes are expected given the design of full-length oligonucleotides. Similarly, B and C barcodes were annotated. The barcode for alpha and omega comes from index read 2 and index read 1, respectively, and was annotated by a custom de-multiplexing script.

A merger of three droplets could contain a single drug (merger of the same drug droplets from replicate wells from plates A, B and C; or one non-zero concentration drug droplet merged with two zero-concentration droplets; etc.), a two-drug combination, or a three-drug combination. Experimental drug concentrations were calculated using input drug quantities, from the decoding of A-B-C barcodes, divided by the calculated average size of cell incubation droplet.

Single agent effect could be evaluated using apoptosis percentage derived from single drug droplet experiments. Synergy and additive effects of two or more drugs are evaluated by a number of statistical models such as Loewe additivity and Bliss independence models using the SAS software.

### Example 3

To allow for improved cell viability during prolonged drug treatments in droplets, and to allow for simpler manipulation of cells following drug treatment, this example illustrates embedding cells in agarose gel prior to drug treatment. Manipulation is simplified since magnetic streptavidin nanobeads can be incorporated into the gels that both bind the drug barcodes and allow for magnetic bead washing and separation, and the post-drug step can be performed outside of lipid droplets in bulk, after destabilizing droplets, allowing for staining and washing.

In this example, first cells are mixed with molten low melt agarose at 37° C. and together incorporated into droplets. See, e.g., Int. Pat. Apl. Pub. No. WO 2008/109176, incorporated herein by reference. An UltraPure low melting point agarose is used in this experiment, which melts at 65.5° C., remains fluid at 37° C. and becomes solid rapidly at temperatures below 25° C. In addition, its gel structure allows for better handling and less breakage. In this example, agarose powder was dissolved into PBS and autoclaved to prepare a sterilized 2% (w/v) agarose solution. Cells were counted and suspend in a DMEM culture media with 40% fetal bovine serum, and the cell suspension was mixed with agarose solution at 1:1 volume ratio. The mixture of cells and agarose gel was flowed into the sample inlet of a microfluidic drop-making device, and HFE7500 oil with 2% surfactant was flowed into the oil inlet to cause the cell-containing agarose gel to produce 80 micrometer drops. The generated gel-in-oil drops were collected into a pre-cooled eppendorf tube in an ice bath.

To further simply the microfluidic manipulation and minimize the time for cells to be exposed to an anaerobic environment when they are put in syringes during encapsulation and re-injection, this example uses a one-step procedure to inject the cell/gel mixture into drops already containing drugs. The drops containing agarose gel particles were collected into a pre-cooled eppendorf tube, after which the injected cell-containing agarose gel remained spherical. In the following steps, the agarose gel particles were injected into the drop containing drug combination and drug barcodes, followed by incubation, destabilization, staining, washing and FACS to obtain the gel particles containing positive cells. To remove oil and surfactant, a drop destabilizer was added to the emulsion and the agarose gel particles were collected by centrifuge.

Leukemia K562 cells having abnormally high activity of oncogene tyrosine kinase(s) and a kinase inhibitor drug, Imatinib, were used as a model, where Imatinib can induce K562 cell apoptosis. The cells were mixed with the agarose gel solution and injected into the mixture into the drops containing 20 micrograms/mL Imatinib at a 1:1 ratio. After 48 hr, the emulsions were burst and the gel particles stained with propidium iodide (PI) and Annexin V to determine the apoptosis and necrosis through differences in plasma membrane integrity and permeability, followed by three rounds of washing.

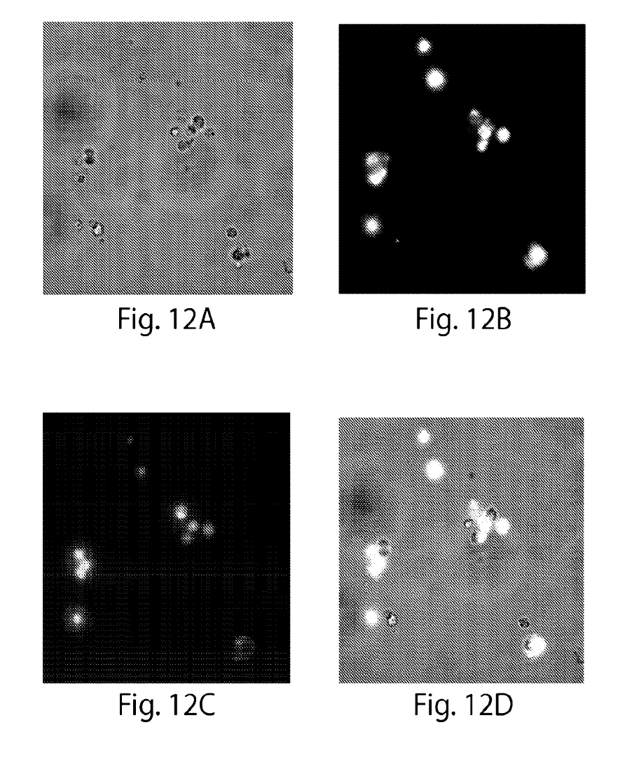

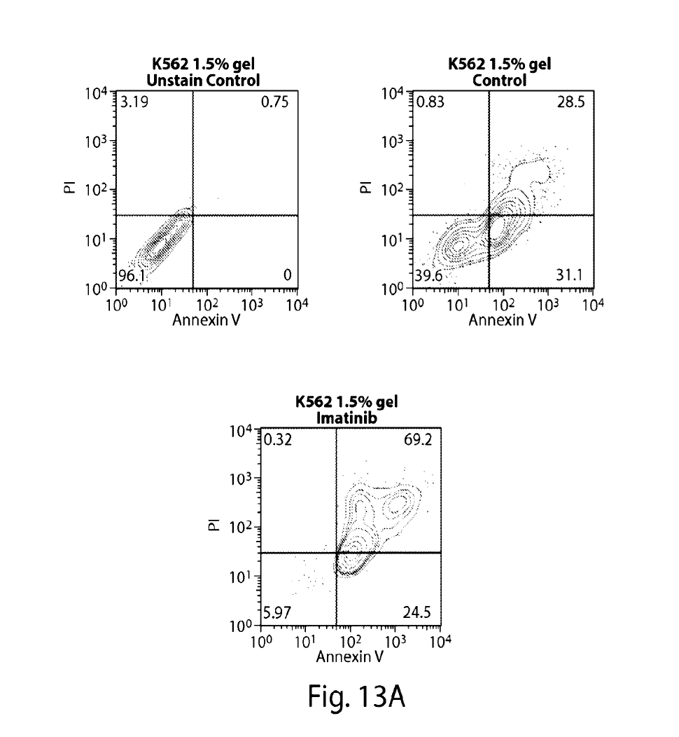

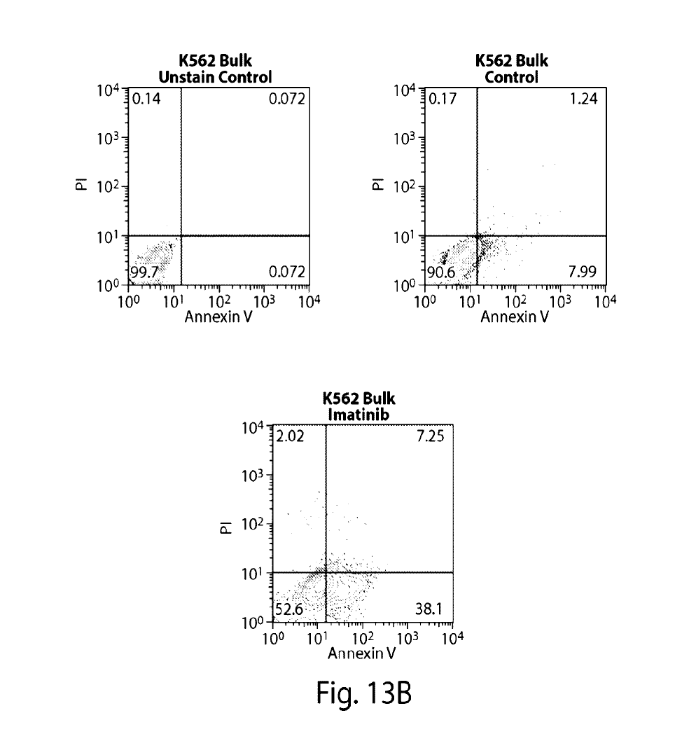

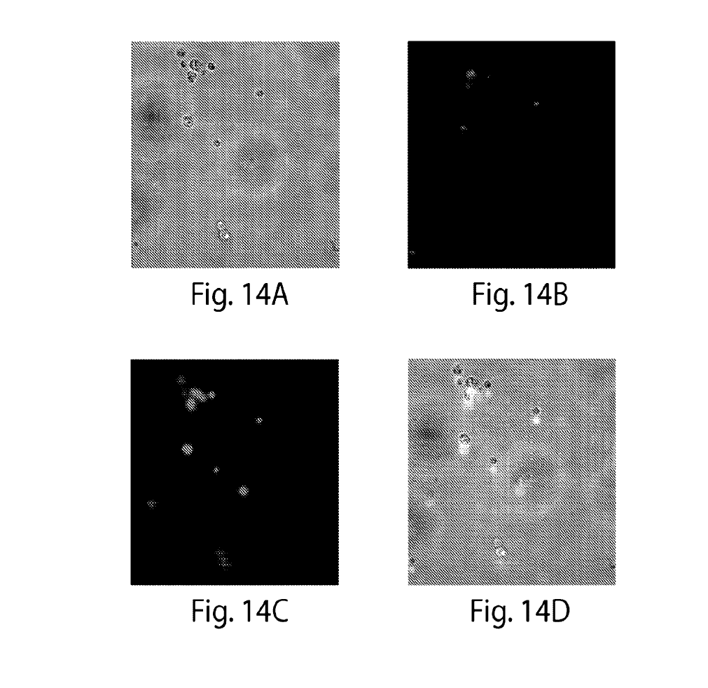

About 70% apoptotic and necrotic cells were observed as shown in FIG. 12. The flow cytometry detection showed more quantitative results (FIG. 13). The cells were also stained with PI and Calcein AM to directly determine the viable and dead cells, as shown in FIG. 14. Thus, this procedure was used to encapsulate cells, culture them in drops, stain them, wash them, and detect them using flow cytometry. Subsequently, FACS or other techniques may be used to sort for positive cells.

FIG. 12 shows fluorescence microscope images of leukemia K562 cells in 1.5% agarose particles after 48 hr of incubation with the presence of 10 micrograms/mL Imatinib. FIG. 12A is bright field; FIG. 12B is the Annexin V channel (green channel); FIG. 12C is the PI channel (red channel); and FIG. 12D is a merged image of FIGS. 12B and 12C.

FIG. 13 shows flow cytometry detection results. FIG. 13A shows leukemia K562 cells in 1.5% agarose particles after 48 hr of incubation with the presence of 10 microgram/mL Imatinib. FIG. 13B shows leukemia K562 cells after 48 hr of incubation with the presence of 10 microgram/mL Imatinib.

FIG. 14 shows fluorescence microscope images of leukemia K562 cells in 1.5% agarose particles after 48 hr of incubation with the presence of 10 microgram/mL Imatinib. FIG. 14A is bright field; FIG. 14B is the calcein AM channel (green channel); FIG. 14C is the PI channel (red channel); and FIG. 14D is a merged image of FIGS. 14B and 14C.

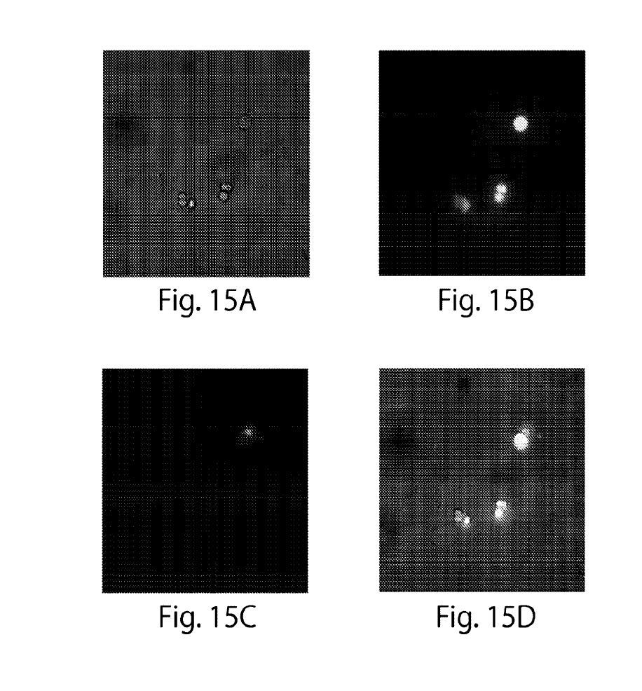

FIG. 15 shows fluorescence microscope images of leukemia K562 cells after 48 hr of incubation with the presence of 10 microgram/mL Imatinib. FIG. 15A is bright field; FIG. 15B is the calcein AM channel (green channel); FIG. 15C is the PI channel (red channel); and FIG. 15D is a merged image of FIGS. 15B and 15C.

### Example 4

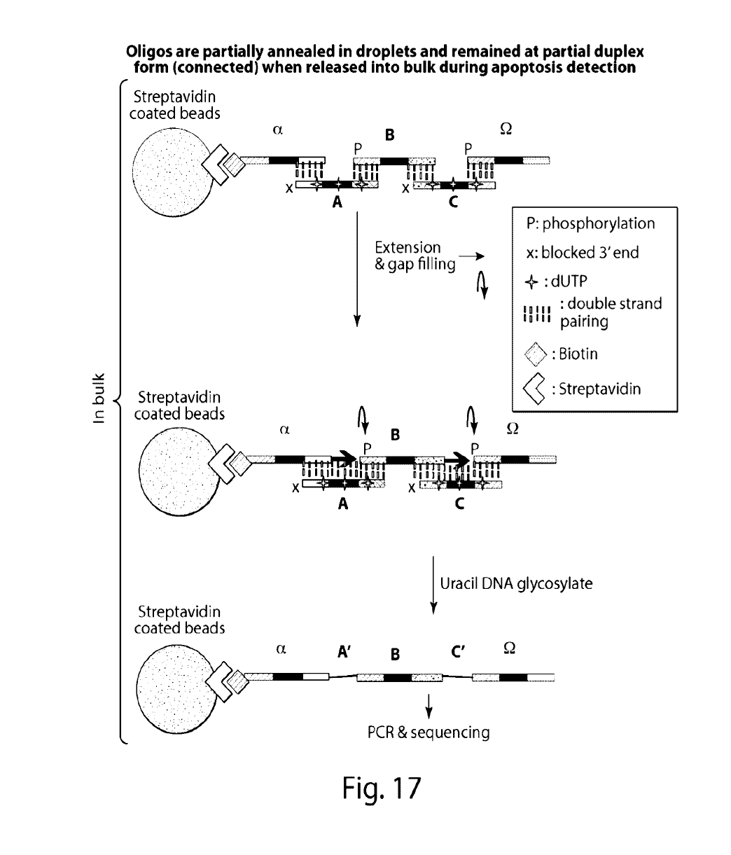

This example illustrates a streptavidin-biotin-based method to establish the linkage between drugs and cells. See also FIG. 17. During drug encapsulation, three barcodes A, B, and C are added into three sets of 96-well plate containing different drugs, respectively, followed by merging the barcodes together. Therefore, each resulting drop contains its unique combinations of both drugs and barcodes. As mentioned, to capture these barcodes on agarose gel particles, nanometer-sized streptavidin magnetic beads are added into agarose gel solution. These beads have been pre-incubated with a biotinylated linker sequence which carries the complementary sequence of barcode A. To test the capture efficiency, agarose gel containing magnetic beads was injected into the drops containing barcodes A, B and C, the drops were collected, the agarose gel was solidified by cooling in ice bath and the droplets incubated at 37° C. for 12 hr. The emulsions were burst and the gel particles collected using a magnetic plate.

With respect to FIG. 17, the extension-and-filling step is achieved in this example using a polymerase without strand displacement property (e.g. Phusion DNA polymerase, NEB) and a gap-filling enzyme that is active (e.g. Ampligase, USB) at the same temperature as the polymerase. The oligonucleotides B and omega (Ω) are 5′ phosphorylated. Oligonucleotides A and C contain dUTP and can be dissolved using uracil DNA glycosylate, to avoid oligonucleotides A and C serving as templates for PCR which would confound the oligonucleotide composition on a same full-length oligonucleotide. After uracil DNA glycosylate treatment, the remaining full-length oligonucleotides are PCR amplified for sequencing.

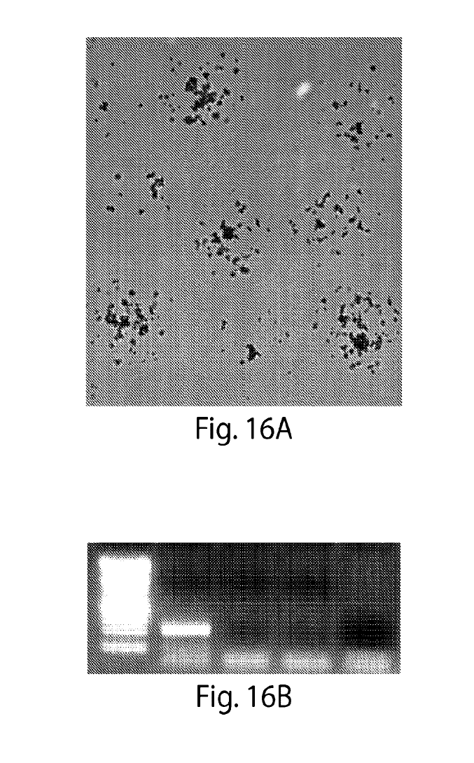

The gel particles are shown in FIG. 16A. These gel particles were used to perform PCR. An expected amplification was observed at 212-bp, which suggested all the barcodes were captured on beads, as shown in FIG. 16B.

FIG. 16A shows an image of nanosized (160-750 nm) streptavidin magnetic beads in agarose gel particles. FIG. 16B shows amplification results of the gel particles. Lane 1, Biotinylated alpha+SA beads, incubated for 5 min, washed and put into agarose gel; lane 2, Biotinylated alpha+SA beads, directly added to agarose gel; lane 3, non-biotinylated alpha+SA beads, incubated for 5 min, washed and put into agarose gel; lane 4, non-biotinylated alpha+SA beads, directly added to agarose gel.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
exposing a microfluidic droplet to a first condition and adding a first nucleic acid to the droplet, wherein the first nucleic acid encodes the first condition;
exposing the microfluidic droplet to a second condition and adding a second nucleic acid to the droplet, wherein the second nucleic acid encodes the second condition; and
amplifying the first nucleic acid and the second nucleic acid within the microfluidic droplet.

2. The method of claim 1, wherein exposing the microfluidic droplet to the first condition and adding the first nucleic acid to the droplet comprises fusing the microfluidic droplet to a second droplet containing the first nucleic acid.

3. The method of any one of claim 1 or 2, wherein exposing the microfluidic droplet to the first condition comprises exposing the droplet to a molecular species.

4. The method of claim 3, wherein exposing the microfluidic droplet to the molecular species comprises fusing the microfluidic droplet with a second droplet containing the molecular species.

5. The method of claim 4, wherein the second droplet further contains the first nucleic acid, whereby when the second droplet is fused to the microfluidic droplet, the first nucleic acid is added to the microfluidic droplet.

6. The method of any one of claim 4 or 5, wherein the second droplet is fused to the microfluidic droplet via dipoles induced in the droplets.

7. The method of any one of claims 4-6, wherein the second droplet is fused to the microfluidic droplet via opposite electric charges placed on the droplets.

8. The method of any one of claims 3-7, wherein exposing the microfluidic droplet to the molecular species comprises injecting the microfluidic droplet with a fluid containing the molecular species.

9. The method of claim 8, wherein the fluid containing the molecular species further contains the first nucleic acid.

10. The method of any one of claims 3-9, wherein the molecular species and the first nucleic acid are added to the microfluidic droplet simultaneously.

11. The method of any one of claims 3-10, wherein the molecular species is added to the microfluidic droplet before the first nucleic acid is added to the microfluidic droplet.

12. The method of any one of claims 3-11, wherein the molecular species is added to the microfluidic droplet after the first nucleic acid is added to the microfluidic droplet.

13. The method of any one of claims 1-12, wherein the first nucleic acid and/or the second nucleic acid comprises a primer sequence.

14. The method of any one of claims 1-13, further comprising immobilizing the first nucleic acid and the second nucleic acid with respect to each other.

15. The method of claim 14, comprising ligating the first nucleic acid to the second nucleic acid.

16. The method of any one of claims 1-15, wherein amplifying the first nucleic acid and the second nucleic acid within the microfluidic droplet comprises using PCR to amplify the first nucleic acid and the second nucleic acid.

17. The method of any one of claims 1-16, wherein amplifying the first nucleic acid and the second nucleic acid comprises producing an amplified nucleic acid comprising the first nucleic acid and the second nucleic acid.

18. The method of any one of claims 1-17, wherein amplifying the first nucleic acid and the second nucleic acid comprises adding a polymerase to the microfluidic droplet.

19. The method of any one of claims 1-18, wherein amplifying the first nucleic acid and the second nucleic acid comprises adding Taq polymerase to the microfluidic droplet.

20. The method of any one of claims 1-19, wherein the microfluidic droplet comprises Taq polymerase.

21. The method of any one of claims 1-20, comprising exposing the microfluidic droplet to a temperature of at least about 50° C.

22. The method of any one of claims 1-21, comprising exposing the microfluidic droplet to a temperature of at least about 90° C.

23. The method of any one of claims 1-22, wherein amplifying the first nucleic acid and the second nucleic acid comprises adding deoxyribonucleotides to the microfluidic droplet.

24. The method of any one of claims 1-23, wherein the microfluidic droplet comprises deoxyribonucleotides.

25. The method of any one of claims 1-24, wherein exposing the microfluidic droplet to the first condition comprises exposing the droplet to an external physical condition.

26. The method of claim 25, wherein exposing the microfluidic droplet to an external physical condition comprises exposing the microfluidic droplet to a predetermined temperature and/or a predetermined pressure.

27. The method of any one of claims 1-26, wherein exposing the microfluidic droplet to the first condition comprises fusing the microfluidic droplet to a second droplet having a pH of less than 7.

28. The method of any one of claims 1-27, wherein exposing the microfluidic droplet to the first condition comprises fusing the microfluidic droplet to a second droplet having pH of greater than 7.

29. The method of any one of claims 1-28, wherein the microfluidic droplet has an average cross-sectional diameter of less than about 1 mm.

30. The method of any one of claims 1-29, wherein the microfluidic droplet is one of a plurality of microfluidic droplets having a distribution of diameters such that no more than about 5% of the microfluidic droplets have a diameter less than about 90% or greater than about 110% of the overall average diameter of the plurality of microfluidic droplets.

31. The method of any one of claims 1-30, further comprising exposing the microfluidic droplet to a third condition and adding a third nucleic acid to the microfluidic droplet, wherein the third nucleic acid encodes the third condition.

32. The method of claim 31, further comprising attaching the third nucleic acid to one or both of the first nucleic acid and the second nucleic acid.

33. The method of any one of claims 1-32, further comprising determining a property of the microfluidic droplet.

34. The method of claim 33, further comprising sorting the microfluidic droplet based on the property.

35. The method of any one of claim 33 or 34, wherein the property is fluorescence.

36. The method of any one of claims 33-35, wherein the property is the average cross-sectional diameter of the microfluidic droplet.

37. The method of any one of claims 33-36, wherein the property is light absorption.

38. The method of any one of claims 33-37, wherein the property is the concentration of an agent contained within the microfluidic droplet.

39. The method of any one of claims 33-38, wherein the property is the condition of a cell contained within the microfluidic droplet.

40. The method of any one of claims 33-39, wherein the cell is contained within a gel.

41. The method of claim 40, wherein the gel is agarose gel.

42. The method of any one of claim 40 or 41, wherein the gel has a solidification temperature of less than about 25° C.

43. The method of claim 39-42, wherein the property is whether the cell is alive or dead.

44. The method of any one of claims 39-43, wherein the property is a concentration of an agent produced and/or consumed by the cell.

45. The method of any one of claims 1-44, further comprising separating the first nucleic acid and the second nucleic acid from the microfluidic droplet.

46. The method of any one of claims 1-45, further comprising bursting the microfluidic droplet.

47. The method of claim 46, wherein the droplet is burst by exposing the droplet to ultrasound.

48. The method of any one of claims 1-47, further comprising sequencing the first nucleic acid and/or the second nucleic acid.

49. The method of any one of claims 1-48, wherein the droplet contains a cell.

50. The method of claim 49, wherein the cell is a human cell.

51. The method of any one of claim 49 or 50, wherein the cell is a cancer cell.

52. The method of any one of claims 49-51, wherein the cell is an immune cell.

53. The method of claim 52 wherein the cell is a bacterial cell.

54. The method of any one of claims 49-53, wherein the cell is a naturally-occurring cell.

55. The method of any one of claims 49-54, wherein the first condition is the cell's type.

56. The method of any one of claims 49-55, wherein the first condition is exposure to a drug suspected of being capable of interacting with the cell.

57. The method of any one of claims 49-56, wherein the first condition is exposure to a suspected drug.

58. The method of claim 57, wherein the drug is cytotoxic.

59. The method of any one of claims 1-58, wherein the droplet is contained within a microfluidic channel.

60. A method, comprising:
exposing a plurality of microfluidic droplets to a plurality of conditions such that substantially each microfluidic droplet is sequentially exposed to at least two different conditions, wherein when a microfluidic droplet is exposed to a condition, a nucleic acid encoding the condition is added to the microfluidic droplet; and
amplifying the nucleic acids within at least some of the microfluidic droplets.

61. The method of claim 60, wherein a nucleic acid encoding a condition is added to a microfluidic droplet by fusing the microfluidic droplet to a second droplet containing the nucleic acid.

62. The method of any one of claim 60 or 61, wherein exposing the plurality of microfluidic droplets to the plurality of conditions comprises exposing at least some of the microfluidic droplets to a molecular species.

63. The method of claim 62, wherein exposing at least some of the microfluidic droplets to a molecular species comprises fusing at least some of the microfluidic droplets with second droplets containing the molecular species.

64. The method of claim 63, wherein the second droplets further contain the first nucleic acid, whereby when the second droplet is fused to the microfluidic droplet, the first nucleic acid is added to the microfluidic droplet.

65. The method of any one of claims 62-64, wherein exposing at least some of the microfluidic droplets to a molecular species comprises injecting the microfluidic droplet with a fluid containing the molecular species.

66. The method of claim 65, wherein the fluid containing the molecular species further contains the first nucleic acid.

67. The method of any one of claims 60-66, wherein a nucleic acid encoding a condition is added to a droplet by fusing the droplet to a second droplet containing the nucleic acid.

68. The method of any one of claims 60-67, wherein a nucleic acid encoding a condition is added to a droplet by injecting a fluid containing the nucleic acid into the droplet.

69. The method of any one of claims 60-68, wherein the at least one of the nucleic acids comprises a primer sequence.

70. The method of any one of claims 60-69, further comprising immobilizing at least some of the nucleic acids within a droplet with respect to each other.

71. The method of any one of claims 60-70, wherein amplifying the nucleic acids comprises using PCR to amplify the nucleic acids.

72. The method of any one of claims 60-71, wherein amplifying the nucleic acids comprises adding a polymerase to at least some of the microfluidic droplets.

73. The method of any one of claims 60-72, wherein amplifying the nucleic acids comprises adding Taq polymerase to at least some of the microfluidic droplets.

74. The method of any one of claims 60-73, wherein at least some of the microfluidic droplets comprise Taq polymerase.

75. The method of any one of claims 60-74, comprising exposing at least some of the microfluidic droplets to a temperature of at least about 50° C.

76. The method of any one of claims 60-75, comprising exposing at least some of the microfluidic droplets to a temperature of at least about 90° C.

77. The method of any one of claims 60-76, wherein amplifying the nucleic acids comprises adding deoxyribonucleotides to at least some of the microfluidic droplets.

78. The method of any one of claims 60-77, wherein at least some of the microfluidic droplets comprise deoxyribonucleotides.

79. The method of any one of claims 60-78, wherein exposing the plurality of microfluidic droplets to the plurality of conditions comprises exposing the plurality of microfluidic droplets to a plurality of external physical conditions.

80. The method of claim 79, wherein exposing the plurality of microfluidic droplets to the plurality of conditions comprises exposing the plurality of microfluidic droplets to a predetermined temperature and/or a predetermined pressure.

81. The method of any one of claims 60-80, wherein the microfluidic droplets have an average cross-sectional diameter of less than about 1 mm.

82. The method of any one of claims 60-81, wherein the plurality of microfluidic droplets have a distribution of diameters such that no more than about 5% of the microfluidic droplets have a diameter less than about 90% or greater than about 110% of the overall average diameter of the plurality of microfluidic droplets.

83. The method of any one of claims 60-82, comprising exposing a plurality of microfluidic droplets to a plurality of conditions such that substantially each microfluidic droplet is sequentially exposed to at least three different conditions.

84. The method of any one of claims 60-83, further comprising determining a property of the microfluidic droplets.

85. The method of claim 84, further comprising sorting the microfluidic droplets based on the property.

86. The method of claim 85, wherein the property is the concentration of an agent contained within the microfluidic droplets.

87. The method of any one of claims 60-86, further comprising separating the nucleic acids from the microfluidic droplets.

88. The method of any one of claims 60-87, further comprising bursting the microfluidic droplet.

89. The method of any one of claims 60-88, further comprising sequencing the nucleic acids within at least some of the microfluidic droplets.

90. The method of any one of claims 60-89, wherein at least one of the plurality of conditions is exposure to a suspected drug.

91. An article, comprising:
a plurality of microfluidic droplets, at least some of the droplets containing one or more primers and one or more nucleic acids encoding a plurality of conditions that the at least some droplets were exposed to.

92. The article of claim 91, wherein the nucleic acids encodes at least three conditions.

93. The article of any one of claim 91 or 92, wherein at least some of the nucleic acids encode molecular species contained within at least some of the microfluidic droplets.

94. The article of claim 93, wherein at least some of the microfluidic droplets further comprise the respective molecular species encoded by the nucleic acids.

95. The article of claim 94, wherein at least some of the microfluidic droplets contains at least two molecular species and nucleic acids encoding the at least two molecular species.

96. The article of any one of claims 91-95, wherein at least some of the microfluidic droplets contains at least three molecular species and nucleic acids encoding the at least three molecular species.

97. The article of any one of claims 91-96, wherein at least some of the microfluidic droplets contains a polymerase.

98. The article of any one of claims 91-97, wherein at least some of the microfluidic droplets contains Taq polymerase.

99. The article of any one of claims 91-98, wherein at least some of the microfluidic droplets are at a temperature of at least about 50° C.

100. The article of any one of claims 91-99, wherein at least some of the microfluidic droplets are at a temperature of at least about 90° C.

101. The article of any one of claims 91-100, wherein at least some of the microfluidic droplets contains deoxyribonucleotides.
